(19) United States
(12) Patent Application Publication
(10) Pub. No.: US 2026/0279015 A1
(43) Pub. Date: Sep. 17, 2026

(54) SYSTEMS AND METHODS FOR HIGH-DIMENSIONAL CONTEXTUALIZATION OF SIGNAL DATA FROM CAPILLARY-DRIVEN ASSAYS USING MACHINE LEARNING ALGORITHMS

(71) Applicant: Edward L. Mamenta, Hudson, FL (US)

(72) Inventor: Edward L. Mamenta, Hudson, FL (US)

(21) Appl. No.: 19/563,225

(22) Filed: Mar. 11, 2026

Related U.S. Application Data
(60) Provisional application No. 63/834,314, filed on Mar. 11, 2025.

(51) Int. Cl. G06V 10/70; G01N 33/543; G06V 10/25; G06V 10/26; G06V 10/62; G06V 10/774; G06V 10/778; H04N 23/51; H04N 23/55

(52) U.S. Cl. CPC G06V 10/768; G01N 33/54388; G06V 10/25; G06V 10/26; G06V 10/62; G06V 10/774; G06V 10/7792; H04N 23/51; H04N 23/55

(57) ABSTRACT

Systems and methods that use machine learning algorithms to perform high-dimensional contextualization of signal data derived from capillary-driven assay devices, thereby enhancing the accuracy, precision, sensitivity, and reliability of these devices. Unitary signal data collected in digital image datasets of reacting capillary-driven assays, such as the image of a test line on a lateral flow assay device or the image of a colored pad on a dry reagent chemistry assay device, is granularly processed into multiple, discrete values contextualized to correlated assay conditions that are measurable in the same dataset. Using machine learning algorithms, the collective set of contextualized values is then used to determine a singular assay result. Additionally includes capillary-driven assay devices optimized to maximize the information available for machine learning analysis, as well as customized assay readers integrating digital cameras and computing systems for processing and interpretation.

### CROSS-REFERENCE TO RELATED APPLICATION

This application claims the benefit of and priority to U.S. Provisional Patent Application No. 63/834,314, filed Mar. 11, 2025, the contents of all of which are incorporated herein by reference.

### FIELD OF THE INVENTION

The present invention relates generally to analytical testing systems and, more particularly, to capillary-driven assay devices and methods that utilize digital imaging and machine learning to analyze assay reactions and improve the accuracy, sensitivity, and reliability of analyte detection and quantification.

### BACKGROUND OF INVENTION

The detection and quantification of chemical and biological analytes in medical, environmental, food safety, and industrial settings rely on analytical assays capable of producing rapid and reliable results. Among these assays, capillary-driven assay systems have become widely used due to their simplicity, low cost, and ability to operate without complex instrumentation. Capillary-driven assays, including lateral flow assays and dry-reagent chemistry tests, rely on the spontaneous movement of liquid samples through porous materials by capillary action. These devices typically contain immobilized reagents that react with a target analyte to produce a result, such as a color change or the formation of a line or band on a membrane.

Capillary-driven assay devices offer several advantages that have made them useful. Because fluid transport occurs through passive forces such as adhesion, cohesion, and surface tension, these assays usually do not require pumps, valves, or other active fluid-control mechanisms. As a result, they can be manufactured inexpensively, are easy to operate, and can provide results within minutes.

Despite these advantages, conventional capillary-driven assays often suffer from limitations in analytical precision and reliability. The passive nature of capillary action means that fluid flow within the device is influenced by numerous factors that can vary between devices or test runs. Differences in membrane pore size, reagent distribution, humidity, sample viscosity, and fluid volume can all affect the movement of liquid and the interaction between reagents and analytes. These variations. can reduce the reproducibility and quantitative accuracy of assay results.

For these reasons, many capillary-driven assays are used primarily for qualitative or semi-quantitative analysis rather than precise measurement of analyte concentration. Conventional analytical approaches typically interpret the assay signal by measuring a single value, such as the average color intensity of a test line or reaction area. However, this approach often fails to account for spatial and temporal variations within the assay reaction, including uneven signal patterns or changes in signal formation over time. Useful information contained in the complex spatial and temporal dynamics of the assay reaction is frequently ignored.

Efforts have been made to improve analytical performance by developing more complex diagnostic tools, such as microfluidic lab-on-a-chip systems that incorporate pumps, valves, and precisely fabricated microstructures to control fluid flow. Although such technologies have shown promise in laboratory research settings, they typically require sophisticated fabrication processes, specialized instrumentation, and higher manufacturing costs. These challenges have limited their widespread adoption where simplicity, affordability, and portability are critical.

Accordingly, there remains a need for improved analytical systems and methods capable of extracting more reliable and quantitative information from simple capillary-driven assay devices without significantly increasing device complexity or cost. In particular, there is a need for approaches that can interpret the spatial and temporal characteristics of assay reactions in a more comprehensive manner, thereby improving the accuracy, sensitivity, and robustness of analyte detection and quantification in capillary-driven assays.

### SUMMARY OF THE INVENTION

There are additional features of the invention that will be described hereinafter and which will form the subject matter of the claims appended hereto. In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

The subject invention discloses a method for determining a quantity of a target substance in a test medium using an assay device having capillary-driven flow, the method comprising: (a) defining, within the assay device, a region of interest (ROI) associated with development of one or more measurable signals during operation of the assay device; (b) partitioning the ROI into a plurality of measurement spatial units, each unit representing a spatial subdivision of the ROI; (c) obtaining, from the plurality of measurement spatial units, a plurality of contextual measurements {Yᵢ}, each contextual measurement Yᵢ representing a measurable property within a corresponding measurement spatial unit and characterizing physical or chemical conditions occurring within the ROI; (d) identifying, from among the contextual measurements {Yᵢ}, one or more signal measurements comprising contextual measurements that include analyte-dependent information arising during operation of the assay device; (e) processing the contextual measurements {Yᵢ} and the signal measurements using a machine-learning model configured to identify patterns or relationships within the measurements and to generate one or more intermediate representations; (f) computing a signal value X for the ROI as a function of the contextual measurements {Yᵢ}, the intermediate representations, and a set of assay-specific parameters C, such that X=f({Yᵢ}, C); and (g) determining a quantity of the target substance in the test medium based at least in part on the computed signal value X.

In embodiments of the subject invention, the assay device comprises a porous or semi-porous material configured to transport the test medium at least in part by capillary action, the porous or semi-porous material including one or more of: a lateral-flow membrane, a dry-reagent chemistry pad, a paper-based microfluidic structure, or a chromatographic medium.

In embodiments of the subject invention, the contextual measurements {Yᵢ} comprise measurements indicative of one or more assay conditions, including at least one of: local fluid transport behavior, reaction kinetics, background or baseline characteristics, membrane wetting dynamics, or variations in physical or chemical properties across the ROI.

In embodiments of the subject invention, the signal measurement comprises a measurement obtained from a measurement spatial unit in which a detectable change occurs in response to the presence or quantity of the target substance in the test medium.

In embodiments of the subject invention, capturing one or more digital images of the assay device during operation of the assay device using a digital camera, the digital images containing data from which the contextual measurements {Yᵢ} and the signal measurements are obtained.

In embodiments of the subject invention, the digital camera captures a single image or a sequence of images recorded at predetermined time intervals during the assay reaction.

In embodiments of the subject invention, the digital camera comprises a complementary metal-oxide-semiconductor (CMOS) image sensor or a charge-coupled device (CCD) image sensor configured to convert incident light from the assay device into digital image data.

In embodiments of the subject invention, the digital camera is integrated into a smartphone configured to record images of the assay device before, during, and after development of the one or more measurable signals within the ROI.

In embodiments of the subject invention, the smartphone is positioned within an assay reader comprising a housing configured to hold both the smartphone and the assay device in fixed relative positions suitable for image capture, the housing including an interior cavity configured to provide a controlled illumination environment.

In embodiments of the subject invention, the housing includes a mirror positioned within the interior cavity and arranged such that the smartphone camera records the assay device through a reflection of the mirror, the mirror facilitating a user-accessible orientation of both the smartphone and the assay device.

In embodiments of the subject invention, the machine learning model comprises one or more of: a convolutional neural network (CNN), a recurrent neural network (RNN), a long short-term memory (LSTM) network, a transformer-based model, a support vector machine (SVM), a random forest, a gradient boosting model, or a hybrid model combining deep learning and classical machine learning components.

In embodiments of the subject invention, the machine learning model is configured to process spatial features, temporal features, or spatiotemporal features extracted from a sequence of digital images of the assay device.

In embodiments of the subject invention, the machine learning model comprises a transformer-based architecture configured to apply attention across spatial or temporal dimensions of the digital images to enhance contextual interpretation of the contextual measurements {Yᵢ} and the signal measurements.

In embodiments of the subject invention, the machine learning model is trained at least in part using a self-supervised learning method that includes selectively masking or occluding portions of one or more regions of the assay device within the training images, thereby enabling the model to infer expected measurements based on contextual relationships within the ROI.

In embodiments of the subject invention, the machine learning model is trained using synthetic training data generated by algorithmically modifying or simulating assay images to introduce variations in signal intensity, flow pattern characteristics, lighting conditions, noise, occlusions, or spatial distortions.

In embodiments of the subject invention, the assay device comprises a lateral flow assay including a nitrocellulose membrane, a sample pad, a conjugate pad, and one or more test regions containing immobilized reagents configured to generate at least one of the signal measurements during capillary-driven transport of the test medium.

In embodiments of the subject invention, the assay device comprises a dry-reagent chemistry assay including one or more porous or absorbent reaction pads containing immobilized reagents configured to undergo a chromogenic, fluorogenic, enzymatic, or colorimetric reaction upon contact with the test medium, the reaction producing one or more signal measurements within the ROI.

In embodiments of the subject invention, establishing a confidence range associated with the determined quantity of the target substance, wherein the confidence range is derived at least in part from an evaluation of prediction accuracy of a control region of the assay device.

In embodiments of the subject invention, the control region comprises a control line of a lateral flow assay, and wherein deviations between predicted values and expected values associated with the control line are used to compute a model confidence score.

In embodiments of the subject invention, the model confidence score is used to dynamically adjust the confidence range associated with one or more signal measurements corresponding to one or more test regions of the assay device.

In embodiments of the subject invention, applying online learning to update parameters of a machine learning model when the model confidence score deviates from expected thresholds.

In embodiments of the subject invention, updating the parameters comprises adjusting model weights or selectively incorporating newly acquired labeled data into a training dataset.

The subject invention further discloses a system for determining a quantity of a target substance in a test medium using an assay device having capillary-driven flow, the system comprising: (a) a digital imaging system configured to record digital image data of at least a region of interest (ROI) of the assay device during development of one or more measurable signals; (b) a processing subsystem configured to partition the ROI into a plurality of measurement spatial units, each spatial unit representing a spatial subdivision of the ROI; (c) the processing subsystem further configured to obtain, from the plurality of measurement spatial units, a plurality of contextual measurements {Yᵢ}, each contextual measurement Yᵢ representing a measurable property within a corresponding spatial unit and characterizing physical or chemical conditions occurring within the ROI; (d) the processing subsystem further configured to identify, from among the contextual measurements {Yᵢ}, one or more signal measurements comprising contextual measurements that include analyte-dependent information arising during operation of the assay device; (e) a machine-learning model stored in memory and executable by the processing subsystem, the machine-learning model being configured to process the contextual measurements {Yᵢ} and the signal measurements to identify patterns or relationships within the measurements and to generate one or more intermediate representations; (f) the processing subsystem further configured to compute a signal value X for the ROI based on the contextual measurements {Yᵢ}, the intermediate representations, and a set of assay-specific parameters; and (g) the processing subsystem further configured to determine a quantity of the target substance in the test medium based at least in part on the computed signal value X.

In embodiments of the subject invention, the assay device comprises a porous or semi-porous material configured to transport the test medium at least in part by capillary action, the porous or semi-porous material including one or more of: a lateral-flow membrane, a dry-reagent chemistry pad, a paper-based microfluidic structure, or a chromatographic medium.

In embodiments of the subject invention, the contextual measurements {Yᵢ} comprise measurements indicative of one or more assay conditions, including at least one of: local fluid transport behavior, reaction kinetics, background or baseline characteristics, membrane wetting dynamics, or variations in physical or chemical properties across the ROI.

In embodiments of the subject invention, the signal measurement comprises a measurement obtained from a measurement spatial unit in which a detectable change occurs in response to the presence or quantity of the target substance.

In embodiments of the subject invention, capturing one or more digital images of the assay device during operation of the assay device using a digital camera, the digital images containing data from which the contextual measurements {Yᵢ} and the signal measurements are obtained.

In embodiments of the subject invention, the digital camera is configured to capture a single image or a sequence of images recorded at predetermined time intervals during the assay reaction.

In embodiments of the subject invention, the digital camera comprises a CMOS or CCD image sensor configured to convert incident light from the assay device into digital image data.

In embodiments of the subject invention, the digital camera is integrated into a smartphone configured to record images of the assay device before, during, and after development of the measurable signals within the ROI.

In embodiments of the subject invention, the assay reader including a housing configured to hold the smartphone and the assay device in fixed relative positions suitable for image capture, the housing including an interior cavity configured to provide a controlled illumination environment.

In embodiments of the subject invention, the housing includes a mirror positioned within the interior cavity and arranged such that the smartphone camera records the assay device through a reflection of the mirror, the mirror facilitating a user-accessible orientation of both the smartphone and the assay device.

In embodiments of the subject invention, the machine learning model comprises one or more of: a CNN, an RNN, an LSTM, a transformer-based model, an SVM, a random forest, a gradient boosting model, or a hybrid model combining deep learning and classical machine learning components.

In embodiments of the subject invention, the machine learning model is configured to process spatial features, temporal features, or spatiotemporal features extracted from the sequence of digital images.

In embodiments of the subject invention, the machine learning model comprises a transformer-based architecture configured to apply attention across spatial or temporal dimensions of the digital images to enhance contextual interpretation of the contextual measurements {Yᵢ} and the signal measurements.

In embodiments of the subject invention, the machine learning model is trained at least in part using a self-supervised learning method that includes selectively masking or occluding portions of one or more regions of the assay device within training images to enable inference of expected measurements based on contextual relationships.

In embodiments of the subject invention, the machine learning model is trained using synthetic training data generated by algorithmically modifying or simulating assay images to introduce variations in signal intensity, flow characteristics, lighting conditions, noise, occlusions, or spatial distortions.

In embodiments of the subject invention, the assay device comprises a lateral flow assay including a nitrocellulose membrane, a sample pad, a conjugate pad, and one or more test regions containing immobilized reagents configured to generate at least one of the signal measurements during capillary-driven transport of the test medium.

In embodiments of the subject invention, the assay device comprises a dry-reagent chemistry assay including one or more porous or absorbent reaction pads containing immobilized reagents configured to undergo a chromogenic, fluorogenic, enzymatic, or colorimetric reaction upon contact with the test medium, producing one or more signal measurements within the ROI.

In embodiments of the subject invention, the processors are further configured to compute a confidence range associated with the determined quantity of the target substance, the confidence range being derived at least in part from a prediction accuracy associated with a control region of the assay device.

In embodiments of the subject invention, the control region comprises a control line of a lateral flow assay and wherein the processors compute a model confidence score based on deviations between predicted values and expected values associated with the control line.

In embodiments of the subject invention, the processors dynamically adjust the confidence range associated with one or more test regions based on the model confidence score.

In embodiments of the subject invention, the processors are configured to update parameters of a machine learning model using online learning when the model confidence score deviates from predetermined thresholds.

In embodiments of the subject invention, updating the parameters comprises modifying stored model weights or selectively incorporating newly labeled data into memory for subsequent training.

The subject invention further discloses methods for determining a quantity of a target substance in a test medium using an assay device having capillary-driven flow, the method comprising: (a) defining, within the assay device, a region of interest (ROI) associated with the development of one or more measurable signals during operation of the assay device; (b) partitioning the ROI into a plurality of measurement patches, each patch representing a spatial subdivision of the ROI; (c) obtaining, from the plurality of measurement patches, a plurality of contextual measurements {Yᵢ}, each contextual measurement Yᵢ representing a measurable property within a corresponding measurement patch and characterizing physical or chemical conditions occurring within the ROI; (d) identifying, from among the contextual measurements {Yᵢ}, one or more signal measurements, each signal measurement comprising a contextual measurement that further includes analyte-dependent information arising during operation of the assay device; (e) computing a signal value X for the ROI, the signal value X being computed as a function f of the contextual measurements {Yᵢ} and a set of assay-specific parameters C, such that X=f({Yᵢ}, C); (f) determining a quantity of the target substance in the test medium based at least in part on the computed signal value X.

In embodiments of the subject invention, the assay device comprises a porous or semi-porous material configured to transport the test medium at least in part by capillary action, the porous or semi-porous material including one or more of: a lateral-flow membrane, a dry-reagent chemistry pad, a paper-based microfluidic structure, or a chromatographic medium.

In further embodiments of the subject invention, the contextual measurements {Yᵢ} comprise measurements indicative of one or more assay conditions, including at least one of: local fluid transport behavior, reaction kinetics, background or baseline characteristics, membrane wetting dynamics, or variations in physical or chemical properties across the ROI.

In additional embodiments of the subject invention, each signal measurement comprises a measurement obtained from a measurement patch in which a detectable change occurs in response to the presence or quantity of the target substance in the test medium.

In other embodiments of the subject invention, further comprising capturing one or more digital images of the assay device during operation of the assay device using a digital camera, the digital images containing data from which the contextual measurements {Yᵢ} and the signal measurements are obtained.

In further embodiments of the subject invention, the digital camera captures a single image or a sequence of images recorded at predetermined time intervals during the assay reaction.

In additional embodiments of the subject invention, the digital camera comprises a complementary metal-oxide-semiconductor (CMOS) image sensor or a charge-coupled device (CCD) image sensor configured to convert incident light from the assay device into digital image data.

In additional embodiments of the subject invention, the digital camera is integrated into a smartphone configured to record images of the assay device before, during, and after development of the one or more measurable signals within the ROI.

In further embodiments of the subject invention, the smartphone is positioned within an assay reader comprising a housing configured to hold both the smartphone and the assay device in fixed relative positions suitable for image capture, the housing including an interior cavity configured to provide a controlled illumination environment.

In additional embodiments of the subject invention, the housing includes a mirror positioned within the interior cavity and arranged such that the smartphone camera records the assay device through a reflection of the mirror, the mirror facilitating a user-accessible orientation of both the smartphone and the assay device.

In further embodiments of the subject invention, further comprising processing the contextual measurements {Yᵢ} and the signal measurements using a machine learning model configured to identify patterns or relationships within the measurements and to generate one or more intermediate representations used in computing the signal value X.

In embodiments of the subject invention, the machine learning model comprises one or more of: a convolutional neural network (CNN), a recurrent neural network (RNN), a long short-term memory (LSTM) network, a transformer-based model, a support vector machine (SVM), a random forest, a gradient boosting model, or a hybrid model combining deep learning and classical machine learning components.

In additional embodiments of the subject invention, the machine learning model is configured to process spatial features, temporal features, or spatiotemporal features extracted from a sequence of digital images of the assay device.

In further embodiments of the subject invention, the machine learning model comprises a transformer-based architecture configured to apply attention across spatial or temporal dimensions of the digital images to enhance contextual interpretation of the contextual measurements {Yᵢ} and the signal measurements.

In other embodiments of the subject invention, the machine learning model is trained at least in part using a self-supervised learning method that includes selectively masking or occluding portions of one or more regions of the assay device within the training images, thereby enabling the model to infer expected measurements based on contextual relationships within the ROI.

In further embodiments of the subject invention, the machine learning model is trained using synthetic training data generated by algorithmically modifying or simulating assay images to introduce variations in signal intensity, flow pattern characteristics, lighting conditions, noise, occlusions, or spatial distortions.

In additional embodiments of the subject invention, the assay device comprises a lateral flow assay including a nitrocellulose membrane, a sample pad, a conjugate pad, and one or more test regions containing immobilized reagents configured to generate at least one of the signal measurements during capillary-driven transport of the test medium.

In further embodiments of the subject invention, at least one test region comprises a test band having a width greater than that of a conventional test line, the test band producing a spatial or temporal signal pattern that contributes both contextual measurements {Yᵢ} and signal measurements usable for predictive interpretation of analyte concentration, and wherein multiple test bands may be provided for a single analyte to generate distinct signal patterns of differing sensitivity or dynamic range.

In other embodiments of the subject invention, the assay device comprises a dry-reagent chemistry assay including one or more porous or absorbent reaction pads containing immobilized reagents configured to undergo a chromogenic, fluorogenic, enzymatic, or colorimetric reaction upon contact with the test medium, the reaction producing one or more signal measurements within the ROI.

In embodiments of the subject invention, the dry-reagent chemistry assay includes multiple reaction zones or regions having differing reagent loadings, sensitivities, or reaction kinetics, the zones producing spatially or temporally distinct signal measurements that contribute to the contextual measurements {Yᵢ} and enable predictive interpretation of analyte concentration.

In additional embodiments of the subject invention, further comprising establishing a confidence range associated with the determined quantity of the target substance, wherein the confidence range is derived at least in part from an evaluation of prediction accuracy of a control region of the assay device.

In further embodiments of the subject invention, the control region comprises a control line of a lateral flow assay, and wherein deviations between predicted values and expected values associated with the control line are used to compute a model confidence score.

In embodiments of the subject invention, the model confidence score is used to dynamically adjust the confidence range associated with one or more signal measurements corresponding to one or more test regions of the assay device.

In further embodiments of the subject invention, the higher prediction accuracy associated with the control line results in a narrower confidence range and wherein a lower prediction accuracy results in a wider confidence range.

In additional embodiments of the subject invention, the relationship between control-line prediction accuracy and the confidence range is calibrated during training using historical data including ground truth measurements.

In further embodiments of the subject invention, further comprising applying online learning to update parameters of a machine learning model when the model confidence score deviates from expected thresholds.

In other embodiments of the subject invention, the parameters comprises adjusting model weights or selectively incorporating newly acquired labeled data into a training dataset.

In additional embodiments of the subject invention, the assay device comprises at least two test regions corresponding to different assay formats including a competition-format region and a sandwich-format region, and wherein the confidence range is determined jointly across the test regions using the model confidence score derived from the control region.

The subject invention further discloses systems for determining a quantity of a target substance in a test medium, the system comprising: (a) an assay device having capillary-driven flow and configured to develop one or more measurable signals during operation; (b) a digital camera configured to capture one or more digital images of at least a region of interest (ROI) of the assay device; (c) one or more processors and memory storing instructions which, when executed by the processors, cause the system to perform the method identified above.

In additional embodiments of the subject invention, the assay device comprises a porous or semi-porous material configured to transport the test medium at least in part by capillary action, the porous or semi-porous material including one or more of: a lateral-flow membrane, a dry-reagent chemistry pad, a paper-based microfluidic structure, or a chromatographic medium.

In further embodiments of the subject invention, the contextual measurements {Yᵢ} comprise measurements indicative of one or more assay conditions, including at least one of: local fluid transport behavior, reaction kinetics, background or baseline characteristics, membrane wetting dynamics, or variations in physical or chemical properties across the ROI.

In other embodiments of the subject invention, the signal measurement comprises a measurement obtained from a measurement patch in which a detectable change occurs in response to the presence or quantity of the target substance.

In additional embodiments of the subject invention, the digital camera is configured to capture a single image or a sequence of images recorded at predetermined time intervals during the assay reaction.

In further embodiments of the subject invention, the digital camera comprises a CMOS or CCD image sensor configured to convert incident light from the assay device into digital image data.

In other embodiments of the subject invention, the digital camera is integrated into a smartphone configured to record images of the assay device before, during, and after development of the measurable signals within the ROI.

In additional embodiments of the subject invention, further comprising an assay reader including a housing configured to hold the smartphone and the assay device in fixed relative positions suitable for image capture, the housing including an interior cavity configured to provide a controlled illumination environment.

In further embodiments of the subject invention, the housing includes a mirror positioned within the interior cavity and arranged such that the smartphone camera records the assay device through a reflection of the mirror, the mirror facilitating a user-accessible orientation of both the smartphone and the assay device.

In additional embodiments of the subject invention, the machine learning model stored in the memory and executable by the processors to process the contextual measurements {Yᵢ} and the signal measurements, the model being configured to identify patterns or relationships within the measurements and to generate intermediate representations used in computing the signal value X.

In further embodiments of the subject invention, the machine learning model comprises one or more of: a CNN, an RNN, an LSTM, a transformer-based model, an SVM, a random forest, a gradient boosting model, or a hybrid model combining deep learning and classical machine learning components.

In other embodiments of the subject invention, the machine learning model is configured to process spatial features, temporal features, or spatiotemporal features extracted from the sequence of digital images.

In additional embodiments of the subject invention, the machine learning model comprises a transformer-based architecture configured to apply attention across spatial or temporal dimensions of the digital images to enhance contextual interpretation of the contextual measurements {Yᵢ} and the signal measurements.

In further embodiments of the subject invention, the machine learning model is trained at least in part using a self-supervised learning method that includes selectively masking or occluding portions of one or more regions of the assay device within training images to enable inference of expected measurements based on contextual relationships.

In embodiments of the subject invention, the machine learning model is trained using synthetic training data generated by algorithmically modifying or simulating assay images to introduce variations in signal intensity, flow characteristics, lighting conditions, noise, occlusions, or spatial distortions.

In additional embodiments of the subject invention, the assay device comprises a lateral flow assay including a nitrocellulose membrane, a sample pad, a conjugate pad, and one or more test regions containing immobilized reagents configured to generate at least one of the signal measurements during capillary-driven transport of the test medium.

In other embodiments of the subject invention, at least one test region comprises a test band having a width greater than that of a conventional test line, the test band producing spatial or temporal signal patterns contributing both contextual measurements {Yᵢ} and signal measurements, and wherein multiple test bands may be provided for a single analyte to generate distinct signal patterns of differing sensitivity or dynamic range.

In further embodiments of the subject invention, the assay device comprises a dry-reagent chemistry assay including one or more porous or absorbent reaction pads containing immobilized reagents configured to undergo a chromogenic, fluorogenic, enzymatic, or colorimetric reaction upon contact with the test medium, producing one or more signal measurements within the ROI.

In additional embodiments of the subject invention, the dry-reagent chemistry assay includes multiple reaction zones having differing reagent loadings, sensitivities, or reaction kinetics, the zones producing spatially or temporally distinct signal measurements that contribute to the contextual measurements {Yᵢ} and enable predictive interpretation of analyte concentration.

In other embodiments of the subject invention, the processors are further configured to compute a confidence range associated with the determined quantity of the target substance, the confidence range being derived at least in part from a prediction accuracy associated with a control region of the assay device.

In further embodiments of the subject invention, the control region comprises a control line of a lateral flow assay and wherein the processors compute a model confidence score based on deviations between predicted values and expected values associated with the control line.

In embodiments of the subject invention, the processors dynamically adjust the confidence range associated with one or more test regions based on the model confidence score.

In other embodiments of the subject invention, the processors are configured to update parameters of a machine learning model using online learning when the model confidence score deviates from predetermined thresholds.

In further embodiments of the subject invention, the updating the parameters comprises modifying stored model weights or selectively incorporating newly labeled data into memory for subsequent training.

In other embodiments of the subject invention, the term “substantially” is defined as at least close to (and can include) a given value or state, as understood by a person of ordinary skill in the art. In one embodiment, the term “substantially” refers to ranges within 10%, preferably within 5%, more preferably within 1%, and most preferably within 0.1% of the given value or state being specified.

### BRIEF DESCRIPTION OF THE DRAWINGS

Advantages of the present invention will be apparent from the following detailed description of embodiments, which description should be considered in conjunction with the accompanying drawings, in which:

FIG. 1 is a flowchart showing a preferred embodiment of an approach for breaking down the total digital image dataset of an assay reaction into fundamental data units that may then be subjected to high dimensional contextualization with machine learning algorithms.

FIG. 2A is a set of selected digital images for a reacting lateral flow assay collected over the course of 120 seconds from the red channel of a smartphone camera.

FIG. 2B is a set of selected digital images for a reacting lateral flow assay collected over the course of 120 seconds from the green channel of a smartphone camera.

FIG. 2C is a set of selected digital images for a reacting lateral flow assay collected over the course of 120 seconds from the blue channel of a smartphone camera.

FIG. 2D is a Table providing the respective time stamp (in seconds) for the selected images shown in FIGS. 2A through 2C.

FIG. 3A is the green channel digital image of a lateral flow assay test line and surrounding area

FIG. 3B is the FIG. 3A image in which various patches of space have been marked and labeled for discussion.

FIG. 3C is a set of three patches shown in FIG. 3B magnified and placed side-by-side for comparison.

FIG. 3D is the FIG. 3A image highlighting an area of the test line exhibiting a gradient signal.

FIG. 4A is the final digital image ROI of a two-minute recording of a lateral flow assay reaction recorded on a smartphone. A 1×3 grid is superimposed over the ROI creating three discrete spatial units.

FIG. 4B is a graph of temporal curves generated over the first 80 seconds of the assay reaction recorded in FIG. 4A, plotting the three spatial units with time on the x-axis and pixel intensity on the y-axis.

FIG. 5A is the FIG. 4A final digital image wherein a 3×10 grid superimposed over the ROI creating 30 discrete spatial units.

FIG. 5B is a graph of temporal curves generated over the first 80 seconds of the assay reaction recorded in FIG. 4A, plotting the 30 spatial units with time on the x-axis and pixel intensity on the y-axis.

FIG. 6 is the final digital image ROI of a two-minute recording of a lateral flow assay reaction recorded on a smartphone. A 39×52 grid is superimposed over the ROI creating 2,028 discrete spatial units. Temporal curves are shown for five selected spatial units.

FIG. 7A shows the digital image of a pair of lateral flow test lines from competitive lateral flow assay devices designed to detect amphetamine qualitatively at a cutoff of 500 ng/ml. The image on the left shows a test line from an assay device reacted with negative sample. The image on the right shows a test line from an assay device reacted with positive sample at a concentration of 20 ng/ml, a concentration that would not be expected to have any effect on the test line.

FIG. 7B is a graph plotting the zonal difference in grayscale values for the test lines shown in FIG. 7A.

FIG. 8A is a set of selected digital images for a dry-reagent chemistry assay collected over the course of 540 seconds from the red channel of a smartphone camera.

FIG. 8B is a set of selected digital images for a dry-reagent chemistry assay collected over the course of 540 seconds from the green channel of a smartphone camera.

FIG. 8C is a set of selected digital images for a dry-reagent chemistry assay collected over the course of 540 seconds from the blue channel of a smartphone camera.

FIG. 8D is a Table providing the respective time stamp (in seconds) for the selected images shown in FIGS. 8A through 8C.

FIG. 9A is the green channel digital image of a dry-reagent chemistry assay with a subregion highlighted.

FIG. 9B is the FIG. 9A image in which the highlighted subregion of 9A is magnified and two patches of space have been marked and labeled for discussion.

FIG. 9C is a set of two patches shown in FIG. 8B magnified and placed side-by-side for comparison.

FIG. 10 is the final digital image ROI of a nine-minute recording (green, red and blue channels) of a dry-reagent chemistry assay reaction recorded on a smartphone. The green channel image is enlarged and a 40×40 grid is superimposed over it creating 1,600 discrete spatial units. A temporal curve is shown for one of the selected spatial units.

FIG. 11A is diagrammatic top view of a lateral flow assay device configured with two test “bands” (wider test lines) and a control band. The test bands comprise a sandwich and a competitive format. The Figure shows the device prior to the addition of sample.

FIG. 11B is the FIG. 11A device following the addition of a negative sample.

FIG. 11C is the FIG. 11A device following the addition of a low positive sample.

FIG. 11D is the FIG. 11A device following the addition of a high sample.

FIG. 12A is diagrammatic top view of a lateral flow assay device configured with two test “bands” (wider test lines) and a control band. The test bands comprise competitive format at different sensitivities. The Figure shows the device prior to the addition of sample.

FIG. 12B is the FIG. 12A device following the addition of a negative sample.

FIG. 12C is the FIG. 12A device following the addition of a low positive sample.

FIG. 12D is the FIG. 12A device following the addition of a high sample.

FIG. 13A is a diagrammatic top view of a lateral flow assay device similar to FIG. 11A with the sample waste component removes and the nitrocellulose membrane extended, allowing for the flow stream to be inferred rather than directly measured. The figure shows the device prior to the addition of sample.

FIG. 13B is the FIG. 13A device following the addition of low positive sample.

FIG. 14A is a diagrammatic representation of a machine learning model making a poor prediction of a test line/band appearance and reporting a low confidence score.

FIG. 14B is a diagrammatic representation of a machine learning model making a good prediction of a control line/band appearance and reporting a high confidence score.

FIG. 15A is a diagrammatic representation of a machine learning model making a poor prediction of a test line/band appearance and reporting a low confidence score.

FIG. 15B is a diagrammatic representation of a machine learning model making a good prediction of a test line/band appearance and reporting a high confidence score.

FIG. 16A is a series of digital images showing the test line results for amphetamine assay strips following serial dilutions of amphetamine.

FIG. 16B is a set of 50 digital images showing the test line results for amphetamine assay strips treated with either 0 ng/ml or 20 ng/ml amphetamine and assembled in random order.

FIG. 17 is a scatter plot showing the results of analyzing the 50 assay strips shown in FIG. 16B and classifying the strips as “positive” or “negative” using machine learning features of the invention.

FIG. 18A is a perspective view of the housing for a smartphone based assay reader configured for reading vertically oriented assay devices.

FIG. 18B is a perspective view of a smartphone based assay reader configured for reading vertically oriented assay devices, with such a device inserted.

FIG. 19A is a diagrammatic front view of the FIG. 18A assay reader housing.

FIG. 19B is a diagrammatic front view of the FIG. 18B assay reader.

FIG. 20A is a diagrammatic side view of the FIG. 18A assay reader housing.

FIG. 20B is a diagrammatic side view of the FIG. 18B assay reader.

FIG. 20C is a diagrammatic cutaway side view of the FIG. 18B assay reader showing the internal mirror.

FIG. 21A is a perspective view of the housing for a smartphone based assay reader configured for reading horizontally oriented assay devices.

FIG. 21B is a perspective view of a smartphone based assay reader configured for reading horizontally oriented assay devices, with such a device inserted.

FIG. 22A is a diagrammatic front view of the FIG. 21A assay reader housing.

FIG. 22B is a diagrammatic front view of the FIG. 21B assay reader.

FIG. 23A is a diagrammatic side view of the FIG. 21A assay reader housing.

FIG. 23B is a diagrammatic side view of the FIG. 21B assay reader.

FIG. 23C is a diagrammatic cutaway side view of the FIG. 21B assay reader showing the internal mirror.

FIG. 24A is a diagrammatic front view of a vertically oriented lateral flow assay device incorporating a detachable trough.

FIG. 24B is a diagrammatic back view of the FIG. 24A assay device.

FIG. 24C is a diagrammatic side view of the FIG. 24A assay device.

FIG. 24D is a diagrammatic front view of the FIG. 24A assay device with the trough detached.

FIG. 24E is a diagrammatic back view of the FIG. 24A assay device with the trough detached.

FIG. 24F is a diagrammatic side view of the FIG. 24A assay device with the trough detached.

FIG. 25A is a diagrammatic front view of a horizontally oriented lateral flow assay device incorporating a detachable trough.

FIG. 25B is a diagrammatic back view of the FIG. 25A assay device.

FIG. 25C is a diagrammatic side view of the FIG. 25A assay device.

FIG. 25D is a diagrammatic front view of the FIG. 25A assay device with the trough detached.

FIG. 25E is a diagrammatic back view of the FIG. 25A assay device with the trough detached.

FIG. 25F is a diagrammatic side view of the FIG. 25A assay device with the trough detached.

FIG. 26A is a diagrammatic side view of the FIG. 18B assay reader showing the FIG. 24A or FIG. 25A assay device in the process of being inserted onto the reader.

FIG. 26B is a diagrammatic side view of the FIG. 26A reader/device after insertion of the device.

FIG. 26C is a diagrammatic side view of the FIG. 26B reader/device showing the process of adding sample to the device.

FIG. 27 is a perspective view of the FIG. 18B assay reader with the FIG. 24A lateral flow assay device inserted and receiving sample into its trough.

FIG. 28A is a diagrammatic front view of the FIG. 27 smartphone showing the recorded image of the FIG. 27 assay reaction 5 seconds after prompting the addition of sample.

FIG. 28B is a diagrammatic front view of the FIG. 27 smartphone showing the recorded image of the FIG. 27 assay reaction 34 seconds after prompting the addition of sample.

FIG. 28C is a diagrammatic front view of the FIG. 27 smartphone showing the recorded image of the FIG. 27 assay reaction 48 seconds after prompting the addition of sample.

FIG. 28D is a diagrammatic front view of the FIG. 27 smartphone showing the recorded image of the FIG. 27 assay reaction 180 seconds after prompting the addition of sample.

FIG. 29A is a diagrammatic top view of a horizontally oriented lateral flow assay device, first embodiment

FIG. 29B is a diagrammatic bottom view of the FIG. 24A assay device.

FIG. 29C is a diagrammatic side view of the FIG. 29A assay device.

FIG. 29D is a diagrammatic top view of a horizontally oriented lateral flow assay device second embodiment.

FIG. 29E is a diagrammatic bottom view of the FIG. 24D assay device.

FIG. 29F is a diagrammatic side view of the FIG. 29D assay device.

FIG. 30A is a diagrammatic top view of a horizontally oriented dry-reagent chemistry assay device incorporating a single sample port.

FIG. 30B is a diagrammatic side view of the FIG. 30A assay device.

FIG. 31 is a diagrammatic bottom view of the FIG. 30A assay device, first embodiment.

FIG. 32 is a diagrammatic bottom view of the FIG. 30A assay device, second embodiment.

FIG. 33 is a diagrammatic bottom view of the FIG. 30A assay device, third embodiment.

FIG. 34 is a diagrammatic bottom view of the FIG. 30A assay device, fourth embodiment.

FIG. 35A is a diagrammatic top view of a horizontally oriented dry-reagent chemistry assay device incorporating three sample ports

FIG. 35B is a diagrammatic side view of the FIG. 35A assay device.

FIG. 35C is a diagrammatic bottom view of the FIG. 35A assay device.

FIG. 36 is a diagrammatic side view of the FIG. 21B assay reader showing a horizontal assay device in the process of being inserted onto the reader.

FIG. 37A is a perspective view of the FIG. 21B reader with a single-port assay device inserted, showing a fluid sample in the process of being added with a sample pipet.

FIG. 37B is a perspective view of the FIG. 21B reader with a single-port assay device inserted, showing a whole blood sample in the process of being added with a blood capillary pipet.

FIG. 38 is a perspective view of the FIG. 37B reader with a triple-port assay device inserted, showing a whole blood sample in the process of being added with a blood capillary pipet.

FIG. 39A is a diagrammatic front view of the FIG. 38 smartphone showing the recorded image of the FIG. 38 assay reaction 5 seconds after prompting the addition of sample.

FIG. 39B is a diagrammatic front view of the FIG. 38 smartphone showing the recorded image of the FIG. 38 assay reaction 35 seconds after prompting the addition of sample.

FIG. 39C is a diagrammatic front view of the FIG. 38 smartphone showing the recorded image of the FIG. 38 assay reaction 65 seconds after prompting the addition of sample.

FIG. 39D is a diagrammatic front view of the FIG. 38 smartphone showing the recorded image of the FIG. 38 assay reaction 95 seconds after prompting the addition of sample.

FIG. 40 is a digital image of a pseudo-dry-reagent chemistry assay for hemoglobin recorded approximately 1 minute after capillary blood sample collection from a finger stick.

FIG. 41A is an example of an ROI grid for a lateral flow assay comprising 3,840 discrete spatial units at pixel resolution, with 64 channels and 60 rows.

FIG. 41B is an example of an ROI grid derived from the FIG. 41A ROI grid in which the channels are consolidated in groups of 4 to produce 16 total channels, resulting in 960 discrete spatial units.

FIG. 42A is a top subsection of the FIG. 41A ROI with a 4×60 frame superimposed over the top four 1×60 channels. This frame defines a processed channel (Channel 1) consisting of sixty 1×4 spatial units formed from those original four channels.

FIG. 42B is the FIG. 42A subsection in which the frame is shifted downward 1 pixel to define a 2^{nd} processed channel (Channel 2).

FIG. 42C is the FIG. 42B subsection in which the frame is shifted downward 1 pixel to define a 3^{rd} processed channel (Channel 3).

FIG. 42D is a bottom subsection of the FIG. 41A ROI showing the frame described in FIGS. 42B and 42C after a full completion of 1-pixel shifts to define the final 61^{st} processed channel (Channel 61).

FIG. 42E is a schematic illustrating 61 channels produced by the process described in FIGS. 42A through 42D. The three channels produced as described in FIGS. 42A through 42C are shown, while Channels 4 through 60 are indicated collectively by a vertical sequence of circles denoting these intervening channels. Channel 61 is shown at the end of the sequence.

FIG. 43 is an example of an ROI grid derived from the FIG. 41A ROI grid in which a non-linear channel is defined, comprised of 60 discrete spatial units.

FIG. 44 is a selected set of 12 temporal curve scatter plots in the single channel of an ROI captured for a lateral flow assay reaction. A map of the channel is shown in the center indicating the position of the spatial unit in the channel for each temporal curve.

FIG. 45A is a graph of 25 temporal curve scatter plots from a single channel of an ROI captured for a lateral flow assay reaction. The temporal curves represent the first 25 spatial units from the inflow side of the ROI to the unit showing peak binding.

FIG. 45B is a graph of 27 temporal curve scatter plots from a single channel of an ROI captured for a lateral flow assay reaction. The temporal curves represent the section of the channel from the spatial unit showing peak binding to a spatial unit in the outflow region of the ROI showing no binding.

FIG. 46A is a section of the final digital image of a lateral flow assay wherein an ROI grid is superimposed over the image. Two spatial units from two separate channels are selected for further analysis, identified as the units blanked out.

FIG. 46B is a graph of the temporal curves from the two spatial units selected from Channel 7 of the ROI described in 46A.

FIG. 46C is a graph of the temporal curves from the two spatial units selected from Channel 14 of the ROI described in 46A.

FIG. 47A is a set of selected digital images for a dry-reagent chemistry assay collected over the course of 200 seconds from a smartphone camera.

FIG. 47B is an enlarged image of the ROI at 200 seconds overlaid with the 15×14 spatial-unit grid. Three spatial units exhibiting distinctly different color-development profiles were selected for detailed analysis; these units are shown blanked out in the image.

FIG. 47C is a graph of the temporal curves from the three spatial units selected as described in FIG. 47B.

FIG. 48A is a diagrammatic view of the initial frame in the recording of a lateral flow assay reaction in which a whole blood sample has been applied and separated plasma is visible and captured in the ROI.

FIG. 48B is the FIG. 48A diagrammatic view expanded to include the whole blood separation pad into the image, which is then captured by a larger ROI.

### DESCRIPTION OF THE INVENTION

The following will describe, in detail, several embodiments of the invention. These embodiments are provided by way of explanation only and thus, should not unduly restrict the scope of the invention. In fact, those of ordinary skill in the art will appreciate upon reading the present specification and viewing the present drawings that the invention teaches many variations and modifications, and that numerous variations of the invention may be employed, used and made without departing from the scope and the spirit of the invention.

The invention provides systems and methods for high-dimensional contextualization of signal data from capillary-driven assay devices using machine learning algorithms. By leveraging advanced data processing techniques, such as deep learning neural network models, in conjunction with novel and surprising assay domain discoveries, the invention significantly enhances the accuracy, precision, sensitivity, and reliability of assay results. Traditionally, the signal data from capillary-driven assay devices, such as the test line on a lateral flow assay device or the color on a dry-reagent chemistry pad, have been analyzed using ‘low-dimensional” analyses, treating assay signals as simple unitary reactions that do not account for variability in assay conditions from device-to-device. In contrast, this invention processes the signal data, particularly data derived from digital imaging, as a set of multiple discrete values governed by multiple correlatingly discrete assay conditions which are co-measured with the signal data, a process which may be characterized as “high-dimensional”. Signal data is captured as an array of grayscale values from digital images and analyzed to identify and contextualize these discrete reactions using contextual information embedded within the same images. This contextualization includes detecting assay conditions linked to the discrete reactions, such as different reagent concentrations and different reaction times, specific to various subregions of the signal space, along with analyte-dependent spatial patterns within the signal data that create unique secondary types of signal values. The invention allows for a sufficiently high level of data granularity, manageable through various machine learning algorithms, and the collective set of discrete values is then utilized to determine a singular, highly reliable assay result. Additionally, the invention encompasses capillary-driven assay devices optimized to maximize the information available for machine learning analysis, as well as customized assay readers integrating digital cameras and computing systems for processing and interpretation.

The term, “analyte” as used herein, refers to a specific substance, compound, or biological entity within a sample that an assay is designed to detect, measure, or quantify. The analyte may be a chemical, biomolecule, microorganism, toxin, contaminant, or other target of interest, depending on the application. In medical and veterinary diagnostics, analytes include, but are not limited to, biomarkers such as glucose, cholesterol, creatinine, insulin, hormones, proteins, and pathogens, which may be detected in biological fluids such as blood, urine, or saliva. In food safety applications, analytes may encompass bacterial contaminants, pesticide residues, foodborne toxins, mycotoxins, allergens, or nutrient components. In environmental testing, analytes may include heavy metals, organic pollutants, volatile compounds, microbial contaminants, pesticides or particulate matter found in air, water, or soil. The term, “assay”, as used herein, refers to a procedure used to measure the presence, amount, or activity of a specific substance, such as a chemical, biomolecule, or microorganism, in a given sample. The measurable response produced by an assay is the assay “signal” and is comprised of “signal data”. The physical component or components on or through which assays are performed may be referred to as “assay devices” which include, for example, lateral flow assay strips, alone and within housings or backings.

The term “capillary action,” as used herein, refers to the spontaneous movement of a liquid through narrow spaces within a porous material or along the surface of a solid due to the interplay of adhesive, cohesive, and surface tension forces. Adhesion between the liquid and the solid surface enables the liquid to spread and adhere to the material, while cohesion between liquid molecules maintains fluid continuity. Surface tension, resulting from cohesive molecular interactions at the liquid-air interface, further influences the extent of liquid movement. Capillary action occurs without the need for external forces, such as pumps or gravity, and is governed by factors including the liquid's surface energy, viscosity, and contact angle with the material.

The term “capillary-driven assay,” as used herein, refers to an assay device, method or system in which the movement of a liquid sample is facilitated primarily by capillary action through a porous or microstructured material, without the need for external pumps or active fluid control mechanisms, such movement initiating and/or propagating the assay reaction. Such assays typically comprise one or more hydrophilic substrates, including but not limited to membranes, paper, or polymer-based materials, that guide the liquid sample through predefined pathways to interact with reagents, enabling the detection or quantification of a target analyte. The rate and extent of fluid movement in a capillary-driven assay are influenced by factors such as the material's pore size, surface energy, and structural geometry, as well as environmental conditions such as humidity and temperature. These assays are commonly employed in point-of-care diagnostics, environmental testing, and rapid screening applications where minimal instrumentation and user intervention are desired. Examples of capillary-driven assays include lateral flow devices which perform immunoassay reactions, and dry-reagent chemistry devices which perform chemical, biochemical and/or enzymatic reactions. Fluid flow designs may incorporate lateral flow, flow-through and dip/submersion formats.

The term “dimension”, as used herein with respect to assays, refers to a distinct parameter, variable, or factor that contributes to the characterization, analysis, or interpretation of assay results. Dimensions define the complexity, scope, and informational depth of an assay. Dimensional factors related to how the assay measures the target analyte include “signal dimensions” which are the analyte-dependent measurable responses of the assay, and “context dimensions” which, as used herein, are additional measurable factors necessary to interpret the signal correctly. For assays that produce colorimetric signals, a signal dimension may be, for example, the total grayscale value over a given time period. A context dimension may be, for example, any measurable or determinable condition/process on which an assay signal relies such as assay reaction time and assay reagent concentration.

The term “high-dimensional contextualization” as used herein with respect to assays, refers to the process of interpreting an assay's signal data by incorporating more than two context dimensions per unit of signal and/or sub-dividing the signal data into multiple sub-signals that may be uniform or in the form of a non-uniform pattern.

The term “machine learning algorithms”, as used herein, refers to a set of computational methods and techniques that enable a system to learn patterns and make predictions or decisions based on data, without being explicitly programmed for each task. These algorithms typically involve statistical models and optimization techniques, which allow the system to improve its performance over time by processing and analyzing large datasets. Through iterative learning processes, machine learning algorithms can identify relationships, trends, and structures within the data, enabling the system to generate accurate predictions, classifications, or recommendations.

The term “machine learning model”, as used herein, refers to a specific instantiation of a machine learning algorithm that has been trained on a dataset to perform a particular task, such as classification, regression, or pattern recognition. The model encapsulates the learned relationships, patterns, and structures from the data, which are used to make predictions or decisions on new, unseen data. During the training process, the model is adjusted through optimization techniques to minimize errors and improve accuracy in its predictions. A machine learning model may consist of various components, including parameters, weights, and a trained network architecture, which collectively define its ability to generalize and perform tasks effectively. Once trained, the machine learning model can be deployed to make autonomous decisions or provide insights in real-time or batch processing environments.

The invention solves a longstanding problem associated with the inability to produce accurate, sensitive and reliable results using simple, inexpensive devices based on capillary-driven assays. Capillary-driven assays, such as lateral flow assays and dry reagent chemistry tests, rely on capillary action to initiate and sustain fluid movement without the need for external forces. Capillary action occurs due to the interplay of surface tension, cohesion, and adhesion forces. Cohesion, the attraction between like molecules, allows liquid molecules to hold together, while adhesion, the attraction between liquid molecules and a solid surface, facilitates fluid wicking through porous materials. Surface tension, a result of cohesive forces at the liquid-air interface, further contributes to the spontaneous movement of liquid through narrow spaces. These assays utilize porous materials, such as nitrocellulose membranes, paper, or hydrophilic polymer matrices, which provide a network of microchannels that guide the liquid sample toward reaction zones. The inherent properties of these materials, including pore size, wettability, and fiber structure, influence the rate and efficiency of fluid transport. As the liquid progresses through the device components, it encounters immobilized reagents that react with specific liquid-soluble analytes, enabling rapid detection. By leveraging capillary action, these assays enable simple, rapid, and instrument-free diagnostics, making them valuable for point-of-care and field applications.

Despite their advantages, capillary-driven assays face limitations, particularly in producing reliable, sensitive and quantitative results due to the inherent variability and unpredictability of capillary action. Because fluid movement in these assays is governed by passive forces—surface tension, cohesion, and adhesion—rather than precise external control, slight variations in material properties can lead to inconsistent assay performance. Factors such as inconsistencies in the porous material's structure (e.g., pore size distribution and hydrophilicity) and contact pressure of overlaid porous materials can alter the rate and volume of fluid flow, leading to uneven reagent rehydration, inconsistent reaction times, and incomplete analyte capture. Additionally, variations in sample viscosity and volume can further exacerbate these inconsistencies, making it difficult to achieve reproducible signal intensities necessary for accurate and reliable quantification. Unlike controlled fluidic systems, which regulate fluid flow using pumps or valves, or manual systems that are operated by highly trained/skilled technicians, capillary-driven assays rely on natural wicking, which cannot be finely tuned or predicted, resulting in analyte-independent signal variability and limiting their use in applications requiring accurate, precise, sensitive and reliable quantitation. Consequently, while these assays are highly effective for qualitative or semi-quantitative detection, they are less suitable for applications demanding high precision and repeatability in measurement.

The demand for rapid, accurate, and reliable onsite testing drove significant investment, beginning as far back as the early 1990s, in the development of lab-on-a-chip (LOC) technologies, specifically those fabricated on silicon or glass substrates using lithographic techniques, advanced microfluidics, pumps, and valves to enable precise fluid control. These LOC devices promised to revolutionize diagnostics by miniaturizing entire laboratory workflows onto a single chip, enabling faster results with lower reagent volumes. However, despite decades of research and substantial commercial investment, LOC technology has failed to achieve widespread adoption due to failed performance, high manufacturing costs, complexity in integrating multiple components, lack of standardization, and challenges in mass production. Additionally, regulatory hurdles, reliability issues in real-world environments, and difficulties in transitioning LOC prototypes from research labs to scalable manufacturing have further contributed to its lack of success. Consequently, while LOC technology remains a subject of academic interest, practical commercial applications have failed to materialize.

Unlike the LOC approach, the invention described herein is not based on the requirement of manufacturing expensive, precision microstructures and micromachinery but rather incorporates simple, inexpensive devices based solely on capillary action. Such an approach would seem unlikely or non-obvious given its essential limitations and the fact that these devices were not originally designed with the intention or expectation of performing at a level beyond qualitative or semi-quantitative. Capillary-driven assays, including lateral flow assays and dry-reagent chemistry assays, typically produce results in the form of observable colorimetric changes in a designated test area. For example, lateral flow devices generate signal data in the form of colored “test lines” across a white porous membrane strip. A typical test line may have dimensions of about 1×4 mm. When the test is used for qualitative (positive/negative) analysis, the composition of this test line simply needs to meet the criteria of being visible or non-visible, either to the naked eye or to a photo-optic instrument. Conversely, for a lateral flow device to function quantitatively, the conventional approach is to measure the average color density of the test line, or a section of this test line, and correlate this density with the concentration of target analyte. Using this approach, quantitative performance relies largely on the color density remaining uniform and constant from strip-to-strip for any given analyte concentration, effectively defining the signal response as a unitary measurement. This requirement has proven highly difficult from a manufacturing perspective, resulting in an extremely small percentage of lateral flow devices being marketed as “quantitative” while also relying on the somewhat subjective use of the term, enabling the labeling of a test as “quantitative” for a particular analyte despite the fact that the test may not pass a formal definition such as the definition used for compliance in a CLIA regulated clinical laboratory. A similar situation exists for rapid dry-reagent chemistry assays leading to very few applications of the platform for quantitative analysis despite the significantly large number of potential assays that would prove highly useful if successfully adopted to such a format.

The invention described herein is comprised of recording the reaction of a capillary-driven assay, using a digital camera based instrument, then subjecting the digital data to a novel form of analysis that may be described as “high-dimensional contextualization (HDC)” accomplished with the use of machine learning algorithms. HDC makes use of the novel and surprising finding that capillary-driven assay signals, which may exhibit a large amount of variability and unpredictability from device-to-device (due to the effects of capillary action as described above), can be accurately and reliably assessed by capturing an assay reaction in the form of a digital image dataset (particularly a time series dataset) using a digital camera, then subdividing the assay signal into smaller sub-signals, such as patches of relatively uniform grayscale values or non-uniform, non-random grayscale patterns, co-measuring certain non-signal data of the assay reaction which define the specific set of assay conditions propagating the patch sub-signal, then contextualizing the patch sub-signal to this additional data (which may thus be referred to as “context data”). While such assay conditions (e.g. reagent concentration, assay incubation times) are well defined, fixed and/or constant in a typical laboratory assay, these conditions generally present as variables in a capillary-driven assay. Consistent with the term “context data”, these variables may be referred to as “context variables”. For lateral flow assays, examples of context variables include 1) The amount of reagent particles released from a conjugate pad, 2) the speed each particle travels from the conjugate pad to the test line region as well as the speed of movement through the test line region, and 3) the localized density of particles traveling through sub-sections of the test line region. For dry-reagent chemistry assays, examples of context variables include 1) saturation dynamics wherein patches of sub-regions within the test area saturate and react before other regions, 2) reagent distribution wherein the hydration process causes dry reagent to solubilize non-uniformly through the reaction pad, 3) reagent hydration wherein a saturated subregion fails to solubilize dry reagent relatively quickly causing non-uniform hydration over time and 4) textural variability within the reaction area due to the nature of the capillary material comprising the reaction matrix.

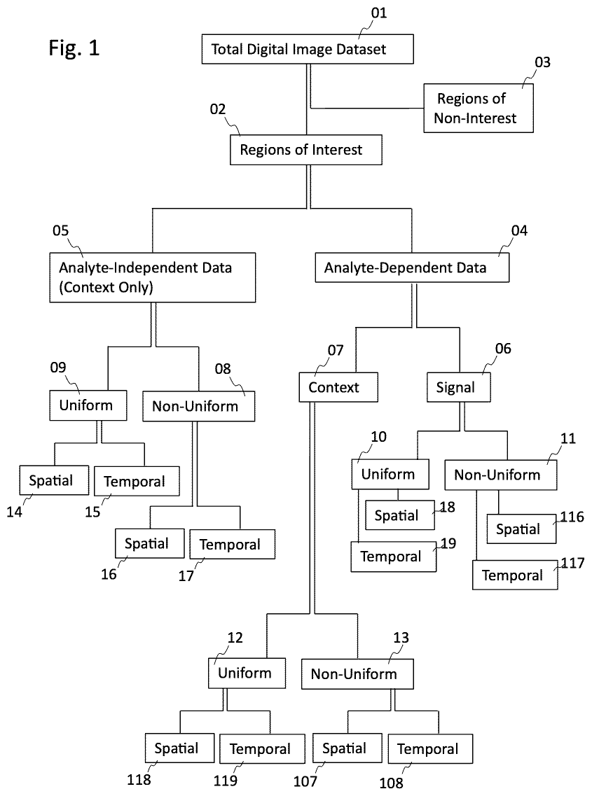

FIG. 1 is a flowchart showing a preferred embodiment of an approach for breaking down the total digital image dataset 01 of an assay reaction into fundamental data units that may then be subjected to high dimensional contextualization with machine learning algorithms. The typical dataset comprises multiple digital images capturing an assay reaction over time with a digital camera, such as through the use of video image capture. The dataset can then be described as a spatial grid of pixel values wherein the grayscale values for a portion of the pixel locations will change over time in accordance with the assay reaction. The dataset may be separated into regions of interest 02 and regions of non-interest 03. The regions of non-interest can be disregarded while the regions of interest may be divided into analyte-independent data 05 and analyte-dependent data 04. Analyte-dependent data is defined as data from the regions of interest that will exhibit variable effects in relation to the concentration of target analyte present in the sample. An example of analyte-dependent data would be the grayscale values for the pixels within the area covering a test line in a lateral flow assay reaction. Another example of analyte-dependent data would be the grayscale values for the pixels within the area covering a color-changing reaction pad in a dry-reagent chemistry assay reaction. These areas of analyte-dependent color changes may also be referred to as “signal area” or “signal space”. Analyte-independent data will be comprised of all data within the regions of interest that are not part of the analyte-dependent data.

The analyte-dependent data is further divided into data related to the assay signal 06 and data related to assay context 07. It should be noted that any subregion of pixels within the analyte-dependent data can be defined as either signal data or context data depending on the particular analytical operation being undertaken, however, a subregion cannot be simultaneously defined as signal and context during the same analytical operation. Analyte-independent data can only be defined as context data as it contains no signal data.

Signal and context data is further divided into uniform (09, 10, 12) and non-uniform (08, 11, 13) data, and each of these are further defined as either spatial (14,16, 18, 116, 118, 107) or temporal (15,17, 19, 117, 119, 108) in nature. For any subregion within the regions of interest, spatial identity is defined on a single image (i.e. an image at any single timepoint) whereas temporal identity is defined over a time series of images. For example, a subregion of 100 pixels (10×10) would be considered spatially uniform on a given image if the grayscale values of the 100 pixels were all relatively equal, having a small standard deviation within a designated threshold. Otherwise, this subregion would be considered spatially non-uniform. If the grayscale values in this subregion remained relatively unchanged over a designated period of time, this subregion would be considered temporally uniform over that period of time. Otherwise, this subregion would be considered temporally non-uniform. The twelve ultimate designations of data that are operational within the methods and systems of the invention underscore the novel “high dimensional” quality of the invention. For comparison, a traditional approach to interpreting capillary-driven assays would rely on only one or two of these data configurations. For example, a dry-reagent chemistry strip may simply measure the overall mean color of a reaction pad at a designated endpoint, irrespective of whether the color was uniform or non-uniform (18 or 116) then compare this to a fixed color reference (14). The minimum uniform spatial subregion is 1 pixel while the minimum non-uniform spatial subregion is 2 pixels. The minimum frame number for a temporal value is 2 while the minimum time interval between frames is the interval within the camera's fastest speed, such as 1/60 second for a speed of 60 fps.

For further clarity, high dimensional contextualization of assay signals may be expressed mathematically and compared with traditional assays that are governed by “low dimensional” processes. For example, the traditional assay may be expressed by the basic equation X=ƒ(Y), wherein X is the target analyte concentration and Y is the unitary signal. For this function to be valid, all assay conditions (i.e. context variables) applied to samples, calibrator samples and control samples must be held constant or fixed. If the collective set of context variables is given as C, a more precise expression of this equation may be written as:

$$X = f ( Y ) for fixed C$$

With the values of C fixed, the equation contains only two variables (the analyte concentration and the unitary signal) which may be regarded as low dimensionality, each variable representing a dimension. In contrast to this low dimensional equation, the high dimensional contextualization method of the present invention may be expressed as:

$$X = f ( \{ Yi \} , C )$$

In this equation, the value X is defined as a function of {Yi}, the weighted average of multiple signals with each “sub-signal” affected by a set context variables specific to that signal. Taking a defined function, such as the equation for a straight line indicative of a linear calibration curve, the traditional assay may be presented as:

$$X = ( Y - b ) / m for fixed C$$

In this equation, m refers to the slope and b refers to the y-intercept. In contrast to this equation, an analogous equation representing a high dimensional contextualization of the signal data may be written as:

$$X = \frac{\sum \text{?} w \text{?} \cdot \frac{Y \text{?} - ( d \cdot C + {d}_{0} )}{( a \cdot C + \text{?} )}}{\sum \text{?} w \text{?}}$$

$$\text{?} \text{indicates text missing or illegible when filed}$$

Whereas the traditional assay is governed by a single measurement related to the signal, the high dimensional assay equation reflects multiple sub-signal measurements, Yᵢ, and multiple measurements of non-signal context data, C, to which the sub-signals are individually contextualized. The factors “a” and “d” in the equation represent coefficients determined experimentally. The factor “wᵢ” represents a weight for each of the contextualized sub-signals which are collectively summarized to produce the final output signal. The weighting is determined experimentally. All experimental determinations may occur during the training phase of a machine learning model.

The invention further incorporates digital cameras to capture one or more digital images of capillary-driven assays over the course of an assay reaction, capturing within these images a dataset processable into signal data and context data. The digital camera is configured into an assay reader designed to efficiently collect digital images from a reacted capillary-driven assay and may be designed as a standalone device built using discrete optical and electronic components or developed as an integrated system leveraging the capabilities of an existing platforms, such as smartphones and laptop computers. The capillary-driven assays are customized for integration with the assay reader and to maximize the analytical capabilities of the machine learning algorithms.

Digital cameras utilize image sensors, including complementary metal-oxide-semiconductor (CMOS) and charge-coupled device (CCD) sensors, to convert incoming light into electrical signals that form digital images. CMOS sensors offer advantages such as lower power consumption and faster readout speeds, while CCD sensors provide high-quality image capture with reduced noise. The camera may be integrated into a smartphone, laptop, or a standalone imaging device, and may operate in still image mode or continuous capture mode to record a sequence of images at predetermined intervals, such as one image per second. The camera typically incorporates one or more light sources, including light-emitting diodes (LEDs), to provide consistent and adjustable illumination. Additional features such as autofocus, optical and digital zoom, infrared capabilities, high dynamic range (HDR), and image stabilization may be employed to optimize image quality under varying environmental conditions. The captured digital images may be stored in internal memory, transmitted wirelessly, or processed in real time using onboard or external computing resources.

In a preferred embodiment, the digital camera incorporated in the invention is supplied by a smartphone. Modern smartphone cameras typically feature image sensors ranging from 8 to 200 megapixels, with high-end models offering large sensor sizes and pixel-binning technology for improved low-light performance. The camera may support frame rates from 24 to 240 frames per second (fps), with standard video recording at 30 or 60 fps and high-speed capture capabilities for slow-motion analysis. Multi-lens configurations, including wide-angle, ultra-wide, telephoto, and macro lenses, allow for variable focal lengths and magnification levels, enabling enhanced detail capture and flexible imaging perspectives. Integrated light sources, such as dual-tone or multi-LED flash units, provide consistent illumination, while advanced features like optical image stabilization (OIS), electronic image stabilization (EIS), and time-of-flight (ToF) sensors enhance clarity and depth perception. Computational photography techniques, including artificial intelligence-based noise reduction, HDR processing, and adaptive exposure control, further optimize image quality. Additionally, smartphones incorporate dedicated image signal processors (ISPs) and neural processing units (NPUs) to facilitate real-time image enhancement and analysis, ensuring that the captured digital images meet the requirements for accurate machine learning-based evaluation within the described machine learning pipeline. Suitable smartphones include the Apple® iPhone® 16 Pro Max, Samsung® Galaxy S25 Ultra, Google® Pixel 9 Pro, Sony® Xperia 1 VI, Xiaomi® 14 Ultra, Huawei® P70 Pro, OnePlus® 12 Pro, Oppo Find X7 Pro, Vivo X200 Pro, and Honor Magic® 6 Pro.

In a preferred embodiment, the smartphone is integrated into an assay reader designed to simplify the testing process and provide a relatively controlled lighting environment in which digital images are captured. The assay reader is comprised of a housing with two positions, one to hold the smartphone and the other to hold an assay device to be recorded by the smartphone. The housing contains an inner cavity.

In a preferred embodiment, the cavity is painted, or otherwise rendered, a matte white color allowing light to bounce off the inner housing surfaces efficiently. The housing incorporates designated locations for both the smartphone and the assay device, oriented in such a way as to allow the smartphone to record images of the assay device before, during and after the device performs an assay reaction. In one embodiment, the assay reader contains a mirror within the cavity allowing for the locations of the smartphone and assay device to be oriented in such a way as to improve the ease-of-use with respect to the manual steps involved in performing the assay. More specifically, the mirror allows for both the smartphone screen and the assay device sample-loading component to be easily observed and accessed while the assay reader remains in a fixed position. For example, assuming the assay reader is placed on a flat level surface such as a tabletop, the smartphone location on the housing will allow the smartphone to be placed at a 45° angle with respect to the table surface, with the camera pointing into the housing at the mirror through an opening in the housing. The assay device may then be placed either perpendicular or parallel to the table surface in proximity of the smartphone such that the assay reaction may be recorded by the smartphone through the reflection of the mirror. In a preferred embodiment, the assay device uses the smartphone light source for illumination of the housing interior. In another preferred embodiment, the assay device uses a separate light source which may be powered by the smartphone or powered independently of the smartphone. In still another preferred embodiment, the assay device measures fluorescence rather than color and incorporates specialized smartphones or other instrument capable of fluorescent measurement.

The invention further incorporates capillary-driven assay devices configured in such a way as to be positioned on/in an assay reader, providing the user easy access to the sample-loading component of the device while simultaneously allowing the camera component of the assay reader to record digital images of the assay reaction on the device.

In a preferred embodiment, the capillary-driven assays are in the form of lateral flow assay devices. Lateral flow assay (LFA) devices utilize a combination of specialized materials and biochemical reagents to enable rapid, sensitive, and specific detection of target analytes. The core structural component of an LFA is the nitrocellulose membrane, a porous substrate that facilitates capillary-driven fluid flow and serves as the medium for the immobilization of capture biomolecules. These membranes are typically backed with plastic support for mechanical stability and feature predefined test and control lines containing immobilized antibodies or other biomolecular receptors. The membrane's porosity and surface chemistry are optimized to ensure efficient wicking of the sample while preventing non-specific binding that could interfere with assay performance.

The conjugate pad, positioned upstream of the nitrocellulose membrane, is responsible for housing labeled detection reagents such as antibodies conjugated to colloidal gold particles, colored latex particles, or fluorescent nanoparticles. Upon introduction of a liquid sample, the conjugate pad releases these reagents, allowing them to interact with the target analyte. The nature of the particle label determines the readout mode of the assay, with colloidal gold yielding visually distinct red or purple lines, while latex or fluorescent nanoparticles facilitate enhanced sensitivity through optical detection methods. The binding affinity and specificity of the labeled antibodies play a crucial role in determining assay performance, with monoclonal antibodies offering high specificity and polyclonal antibodies providing broader epitope recognition.

LFAs are designed in various assay formats, primarily including the sandwich and competition formats. In the sandwich format, commonly employed for detecting large analytes such as proteins, the analyte binds to both the labeled detection antibody and the capture antibody immobilized on the test line, forming a “sandwich” complex that generates a signal proportional to analyte concentration. In contrast, the competition format, used for small molecules such as drugs and toxins, involves competitive binding between the analyte and a labeled analog for limited binding sites on the test line, where the presence of the target analyte results in signal reduction. These formats can be further adapted for multiplexing by incorporating multiple test lines targeting distinct analytes, enabling simultaneous detection of multiple biomarkers within a single test strip.

The control line, located downstream of the test line, serves as an internal quality control feature to confirm proper fluid migration and reagent functionality. This line typically consists of an immobilized secondary antibody or binding protein that captures unbound conjugate particles, ensuring that a visible control signal appears regardless of the presence or absence of the target analyte. Optimized assay conditions, including buffer composition, blocking agents, and wicking rates, are critical to achieving consistent and reproducible performance across different testing conditions.

Lateral flow assay technology is widely applicable across numerous fields, enabling the detection of a diverse range of analytes. These include infectious disease antigens, such as viral and bacterial markers; antibodies indicative of immune response; drugs of abuse and therapeutic drug monitoring compounds; environmental contaminants including pesticides and heavy metals; foodborne pathogens and allergens; metabolic biomarkers such as glucose and ketones; hormones including pregnancy and fertility markers; veterinary diagnostics; and biowarfare agents for defense applications. The adaptability of LFA technology to different target analytes, combined with its rapid and user-friendly format, makes it an essential tool in point-of-care diagnostics, field testing, and on-site monitoring applications.

The invention further comprises the use of lateral flow assays customized to take advantage of the novel analytical procedures provided by the invention. In a preferred embodiment, the test and control lines of the lateral flow assays are broadened from “lines” into “bands” to better accommodate the analysis of gradient patterns. In another preferred embodiment, multiple test lines or bands are provided on a single strip so as to provide predictive patterns that may be used for self-supervised learning, online learning and/or determination of confidence scores indicating a measure of quality control for the assay. In one example, a large molecule analyte may have two lines or bands, one configured in a sandwich assay format and the other configured in a competition assay format. In another example, a small molecule analyte may have two lines or bands, both configured in a competition assay format wherein one band or line is comprised of a relatively high level of binding reagent (low sensitivity zone) and the other band or line is comprised of a relatively low level of binding reagent (high sensitivity zone), such that within the dynamic range of the assay the low sensitivity zone will continue to present a band or line at concentrations in which the band or line completely disappears in the high sensitivity zone. By configuring a test strip with three binding zones as described in the two examples, there will be predictive measurable patterns that do not rely on foreknowledge of the sample analyte concentration and thus can be analyzed as an “unlabeled” dataset.

In another preferred embodiment, the capillary-driven assays are in the form of dry-reagent chemistry assay devices. Dry-reagent chemistry assays are designed for rapid testing applications by utilizing pre-deposited, stabilized reagents on solid-phase reaction pads. These tests function without requiring liquid reagents, as the necessary chemical components are stored in a dry state and activated upon contact with a sample. Reaction pads are typically composed of porous or absorbent materials such as cellulose, glass fiber, or polymeric membranes, which provide controlled wicking and reaction kinetics. The reagents dried onto these pads can include colorimetric indicators, enzymes, co-factors, and other biomolecules necessary for target analyte detection. Despite the term dry-reagent “chemistry”, these assays often incorporate biologically active reagents, including oxidases, peroxidases, dehydrogenases, and immunochemical components, enabling highly specific and sensitive analyses.

Various test designs exist within dry-reagent chemistry assays, including flow-through, dipstick, and lateral flow configurations. Flow-through designs employ a structured membrane or porous substrate where the sample is applied, allowing it to flow through pre-embedded reagent zones, triggering the desired reaction. Dipstick assays involve immersion of a test strip into a liquid sample, where reagent zones sequentially react as the sample migrates through capillary action. Lateral flow formats integrate multiple reaction zones along a membrane, enabling continuous and controlled sample migration to produce visual or instrument-read results. Each design optimizes reagent stability, sample interaction, and result interpretation for specific testing applications.

A representative example of a dry-reagent chemistry test designed for whole blood analysis includes a lateral flow-based assay incorporating a red blood cell separation matrix. In this design, a sample is applied to a sample pad containing a plasma-separating membrane, which selectively retains red blood cells while allowing plasma to migrate forward. The separated plasma then enters a reaction zone containing reagents such as dried enzymes and chromogenic substrates, where it interacts with the reagents to produce a measurable colorimetric response. This configuration is useful, for example, with blood glucose testing, where glucose oxidase and peroxidase are immobilized on the reaction pad to facilitate a color change proportional to glucose concentration.

Dry-reagent chemistry assays have diverse applications across medical, environmental, and industrial sectors. Examples include glucose monitoring in diabetic care, urine metabolite testing for kidney function assessment, blood gas and electrolyte analysis, and rapid infectious disease screening. Beyond medical diagnostics, these assays are utilized in food safety testing, water quality monitoring—including pool and drinking water assessments—and forensic toxicology. The robustness and convenience of dry-reagent chemistry assays make them integral to point-of-care diagnostics, on-site environmental assessments, and other applications requiring rapid, reliable testing in decentralized settings.

The invention further incorporates the use of machine learning algorithms to analyze the digital images recorded by the assay reader or digital camera. Machine learning algorithms are computational models designed to identify patterns, relationships, and trends within data, enabling automated decision-making and predictive analysis. These algorithms can be broadly categorized into supervised learning, where models are trained on labeled data to map inputs to known outputs, and unsupervised learning, where patterns are inferred without predefined labels. Additionally, reinforcement learning techniques may be employed to optimize decision-making through iterative feedback. The invention leverages a range of machine learning models, including deep learning architectures such as convolutional neural networks (CNNs) for spatial feature extraction, recurrent neural networks (RNNs) and long short-term memory (LSTM) networks for sequential data processing, and transformer-based models for capturing long-range dependencies in spatiotemporal data. Traditional machine learning methods, such as support vector machines (SVMs), random forests, and ensemble learning techniques, may also be integrated for feature selection, classification, and regression tasks. By utilizing a combination of these algorithms, the invention effectively processes digital image data to extract meaningful insights, adapt to varying conditions, and improve predictive accuracy.

The invention can use a variety of machine learning models to accomplish all or part of the process including, but not limited to, CNN, RNN/LSTM, Hybrid CNN+Transformer, SVM, Random Forest, Gradient Boosting Machines (GBM), XGBoost, LightGBM, k-Nearest Neighbors (k-NN), Decision Trees, Logistic Regression, Naive Bayes, ElasticNet, Support Vector Regression (SVR), Multi-Layer Perceptrons (MLPs), Bayesian Networks, Hidden Markov Models (HMM), Restricted Boltzmann Machines (RBM), Autoencoders, GANs (Generative Adversarial Networks), Reinforcement Learning (RL) models, Self-Organizing Maps (SOM), k-Means Clustering, Hierarchical Clustering, Gaussian Mixture Models (GMM), and hybrid models combining classical machine learning techniques with deep learning approaches. Additionally, custom models or architectures designed to explicitly account for the characteristics of the problem, such as specialized attention mechanisms, spatiotemporal models, or context-aware models, could be implemented to enhance model performance and prediction accuracy.

The overall strategy for designing a model involves leveraging a combination of spatial and temporal data analysis and machine learning techniques to predict and interpret specific characteristics within a dynamic system captured through series of digital images including digital video. The model must account for variations in the data across multiple dimensions, including temporal shifts, spatial patterns, and contextual interactions between different elements of the system. To address this, the model would first process the data to extract relevant features related to color intensity, movement patterns, and other observable attributes, while also considering the influence of external factors such as lighting conditions or underlying mechanisms that impact the system's behavior. The model would be trained using labeled examples that capture various conditions, and once trained, it would be tasked with accurately predicting outcomes based on new, unlabeled datasets. The labeling, for example, may comprise the numerical value representing the concentration of target analyte in a sample initiating and propagating the recorded assay reaction.

The design of the model would integrate advanced techniques such as convolutional networks for image-based analysis, recurrent networks for handling temporal dependencies, and attention or transformer-based mechanisms to focus on important contextual information across the image frames. By incorporating these approaches, the model would strive to generalize its learning across various scenarios while minimizing the need for extensive retraining, enabling effective predictions even under changing conditions or novel settings.

In one embodiment, the invention uses the following strategy: processing digital video data to extract spatial and temporal features relevant to the system's behavior, including color intensity, movement patterns, and changes in these attributes over time. A combination of deep learning models is employed, such as convolutional neural networks (CNN) for spatial feature extraction, recurrent neural networks (RNN) or long short-term memory (LSTM) networks for temporal dependency modeling, and hybrid models incorporating transformers for enhanced contextual attention across the video frames. Additionally, the system may utilize classical machine learning approaches like support vector machines (SVM) and random forests to complement deep learning techniques, particularly in feature selection and ensemble decision-making. The model is trained on labeled data capturing various conditions, with the ability to generalize new, unseen scenarios by leveraging transfer learning. In some cases, self-supervised learning may be incorporated to improve the model's adaptability to unlabeled data. Further, the system may employ methods such as uncertainty quantification to assess prediction confidence and improve reliability. The resulting model can predict outcomes based on dynamic video data, enabling accurate analysis of system behavior under varying environmental and operational conditions.

In a preferred embodiment, the training method also uses self-supervising techniques to enhance model robustness and generalization by selectively concealing portions of the control line and/or test lines during training. By partially or completely occluding the control line, the model is trained to infer expected properties of the control line based on the characteristics of the flow pattern within the region of interest, thereby reinforcing its ability to accurately account for variables such as total flow stream volume and velocity. Similarly, occluding portions of one or both test lines compels the model to develop an internal representation of the relationship between the flow stream and the test line responses, enabling it to predict missing data based on learned correlations. In some instances, randomized masking techniques are applied across different training examples to ensure the model does not overfit to specific patterns but instead generalizes to unseen variations. Additionally, contrastive learning techniques may be incorporated, wherein the model learns to distinguish between expected and anomalous relationships within the flow pattern and line responses. This self-supervising methodology reduces dependency on fully labeled data, improves resilience to incomplete or occluded visual information, and strengthens the model's ability to make accurate predictions under real-world conditions.

In a preferred embodiment, the training method further incorporates synthetic data generation to augment the dataset and improve model performance across a range of conditions. Synthetic training examples may be created by algorithmically modifying real digital images of the flow pattern within the region of interest, or by generating entirely simulated flow pattern sequences using procedural or physics-based models. These modifications can include adjusting the intensity and distribution of the test lines and control line, altering flow pattern density and velocity, introducing controlled variations in lighting and noise conditions, and applying transformations such as warping, occlusion, and color perturbation. By generating synthetic variations that encompass a broader range of possible real-world scenarios, the model is trained to be more robust against variations in environmental factors, sensor artifacts, and unpredictable distortions. Additionally, synthetic data can be used to balance the dataset by ensuring equal representation of flow pattern conditions across all relevant scenarios, reducing bias and improving generalization. In some implementations, generative adversarial networks (GANs) or other deep learning-based generative models may be employed to produce highly realistic synthetic training data that closely mimics real-world observations. This synthetic data strategy enhances the model's ability to accurately interpret the test lines and control line, even under conditions that were not explicitly present in the original training dataset.

The above description of machine learning algorithms focus on the area of training and inference which fit into an overall systematic framework for processing, analyzing, and interpreting data through a series of structured steps that enable the effective training and deployment of machine learning models. Such a framework may be referred to as a “machine learning pipeline” and can be outlined as four basic steps. The first step, data collection, involves capturing digital recordings of the relevant process under controlled conditions, ensuring that the acquired data encompasses a comprehensive range of potential variations. The second step, data representation and transformation, converts raw data into structured formats suitable for machine learning by applying predefined strategies. This step enhances the interpretability and usability of the data by preserving spatial and temporal relationships while reducing noise and redundancy. The third step, model training and inference, as described above, utilizes the transformed data as input to one or more machine learning models, which may include deep learning architectures such as convolutional neural networks (CNNs), recurrent neural networks (RNNs), transformers, support vector machines (SVMs), decision trees, or hybrid approaches combining multiple techniques. These models learn patterns and relationships within the data to generate predictive outputs. The fourth step, output generation and interpretation, processes the model's predictions into meaningful results, which may include classifications, quantitative estimates, probability distributions, confidence intervals, or other relevant metrics. This step may also involve post-processing techniques to refine predictions and improve interpretability. The structured approach of the Machine Learning Pipeline enables efficient and accurate extraction of predictive insights from digital data, facilitating robust and scalable machine learning applications.

To capture digital recordings as part of the data collection step described above, the invention incorporates a digital camera. Digital cameras utilize image sensors, including complementary metal-oxide-semiconductor (CMOS) and charge-coupled device (CCD) sensors, to convert incoming light into electrical signals that form digital images. CMOS sensors offer advantages such as lower power consumption and faster readout speeds, while CCD sensors provide high-quality image capture with reduced noise. The camera may be integrated into a smartphone, laptop, or a standalone imaging device, and may operate in still image mode or continuous capture mode to record a sequence of images at predetermined intervals, such as one image per second. The camera typically incorporates one or more light sources, including light-emitting diodes (LEDs), to provide consistent and adjustable illumination. Additional features such as autofocus, optical and digital zoom, infrared capabilities, high dynamic range (HDR), and image stabilization may be employed to optimize image quality under varying environmental conditions. The captured digital images may be stored in internal memory, transmitted wirelessly, or processed in real time using onboard or external computing resources.

In a preferred embodiment, the camera is supplied by a smartphone and incorporates advanced imaging specifications to ensure high-quality image capture for analysis. Modern smartphone cameras typically feature image sensors ranging from 8 to 200 megapixels, with high-end models offering large sensor sizes and pixel-binning technology for improved low-light performance. The camera may support frame rates from 24 to 240 frames per second (fps), with standard video recording at 30 or 60 fps and high-speed capture capabilities for slow-motion analysis. Multi-lens configurations, including wide-angle, ultra-wide, telephoto, and macro lenses, allow for variable focal lengths and magnification levels, enabling enhanced detail capture and flexible imaging perspectives. Integrated light sources, such as dual-tone or multi-LED flash units, provide consistent illumination, while advanced features like optical image stabilization (OIS), electronic image stabilization (EIS), and time-of-flight (ToF) sensors enhance clarity and depth perception. Computational photography techniques, including artificial intelligence-based noise reduction, HDR processing, and adaptive exposure control, further optimize image quality. Additionally, smartphones incorporate dedicated image signal processors (ISPs) and neural processing units (NPUs) to facilitate real-time image enhancement and analysis, ensuring that the captured digital images meet the requirements for accurate machine learning-based evaluation within the described machine learning pipeline. Suitable smartphones include the Apple® iPhone® 16 Pro Max, Samsung® Galaxy S25 Ultra, Google® Pixel 9 Pro, Sony® Xperia 1 VI, Xiaomi® 14 Ultra, Huawei® P70 Pro, OnePlus® 12 Pro, Oppo Find X7 Pro, Vivo X200 Pro, and Honor Magic® 6 Pro.

In a preferred embodiment of data representation and transformation described above, the invention employs an approach that may be referred to as a “spatiotemporal attention strategy” which treats the entire dataset as a unified spatiotemporal sequence and applies attention mechanisms to dynamically focus on relevant regions. This approach uses a transformer-based model to learn attention weights across the combined space-time matrix. For example, with lateral flow assays the model learns contextual dependencies between the signal areas of the test lines and control lines without predefining relationships. The attention scores provide interpretability, indicating which parts of the flow stream most influence the signal areas. This strategy is highly flexible and avoids the potential information loss of fixed preprocessing methods, allowing the model to focus on critical temporal shifts and spatial interactions that may otherwise be overlooked

In another preferred embodiment of data representation and transformation, the invention uses an approach that may be referred to as a “hybrid multi-resolution strategy”, wherein different scales of the image data are processed separately to capture both local and global patterns effectively. The image is decomposed into multiple resolutions using techniques such as Gaussian pyramids or wavelet transforms. High-resolution grids are used to analyze fine-grained motion and localized density variations, while low-resolution grids capture global movement patterns and overall density shifts across the sequence. The resulting multi-scale representations are processed separately and then fused in a model to provide a robust understanding of the signal and context data dynamics. This strategy improves computational efficiency by allowing coarse-scale features to guide the finer details, while still capturing both local and global interactions.

In another preferred embodiment of data representation and transformation, the invention employs an approach that may be referred to as a “recurrent grid strategy”, wherein a grid is superimposed on the region of interest and each square of the grid is treated as an individual temporal sequence modeled by a recurrent architecture, such as LSTMs or GRUs. The grayscale changes in each grid square are modeled over time, with each grid square having its own recurrent unit that tracks the temporal evolution of intensity. The final representation of the data encodes spatiotemporal dependencies across the entire grid. This approach preserves the spatial structure of the grid and allows for a more localized treatment of each location before merging information from the entire region of interest. The recurrent grid strategy also facilitates adaptive temporal weighting when integrated with attention mechanisms, improving the overall accuracy of the system.

In another preferred embodiment of data representation and transformation, the invention uses an approach that may be referred to as a “flow-based optical processing strategy”, which directly estimates the motion dynamics of the region of interest instead of relying solely on intensity changes. Optical flow methods, such as the Farneback or Lucas-Kanade algorithms, are applied to estimate per-pixel velocity and direction of movement within the region of interest. These motion vectors are used as additional features alongside the grayscale intensities, helping the model to disambiguate overlapping density effects and improve prediction accuracy. This approach is particularly beneficial in low-density areas where grayscale intensity alone may be insufficient for accurate density estimation. Optical flow techniques can be integrated with convolutional neural networks (CNNs) or transformer-based models to enable end-to-end learning of motion-aware features.

In another preferred embodiment of data representation and transformation, the invention employs an approach that may be referred to as a “diffusion-based density modeling strategy”, where movement is treated as a continuous density flow process governed by diffusion equations. The grayscale intensity in each square is modeled as evolving according to partial differential equations (PDEs), with diffusion parameters learned from the training data. For example, with lateral flow assays the model would predict the test line intensities based on these learned dynamics, utilizing physics-informed neural networks (PINNs) to incorporate physical constraints into the learning process. This strategy allows the model to generalize better to new conditions by capturing the underlying movement physics and provides a continuous representation of density flow, making it robust to variations in resolution or frame rate.

In another preferred embodiment of data representation and transformation, the invention employs an approach that may be referred to as a “temporal curve strategy” in which a grid is superimposed over the region of interest dividing the region of interest into discrete spatial units and, for each unit within the grid, a temporal curve is generated by calculating the average grayscale intensity at each time point over a sequence of digital image frames, with time represented on the x-axis and grayscale intensity on the y-axis. The resulting temporal curves capture spatial and temporal dynamics within the region of interest. For example, with lateral flow assays, units outside the test and control lines generate context curves, which represent the density and velocity of the flow stream, while units within the test and control lines produce signal curves, which correspond to localized binding activity. These signal curves are analyzed in relation to the surrounding context curves, with each context curve weighted according to its influence on the corresponding signal curve. Signal curves may also function as context curves when they are not being evaluated as signal curves. This weighting accounts for the spatial positioning of the squares, such that, for example, a context curve in the uppermost row has a more significant impact on signal curves within the same row than on those in the lowermost row. This strategy enables the model to explicitly encode motion patterns and density trends over time, facilitating the detection of signal relationships while preserving the temporal evolution of the grayscale intensity. The temporal curve strategy can be used in combination with other preprocessing techniques, including convolutional or recurrent architectures, to enhance the predictive accuracy of machine learning models.

In another preferred embodiment of data representation and transformation, the invention employs an approach that may be referred to as a “ROI concatenation strategy” in which a. grid is superimposed over each image frame region of interest, dividing the region of interest into discrete spatial units and concatenated into a single composite grid. This transformation aligns the time dimension spatially, converting the temporal sequence into an extended spatial representation. This transformation enables direct spatial comparison of the temporal evolution of the grayscale intensity across the region of interest. Each column or row, depending on the orientation of concatenation, encodes the grayscale distribution at a particular time step, allowing the model to analyze temporal patterns as spatial structures. By restructuring the data in this manner, the ROI concatenation strategy facilitates the use of spatial feature extraction techniques, such as convolutional neural networks (CNNs), to analyze temporal trends without requiring explicit recurrent architectures. The concatenated representation can be leveraged independently or in conjunction with additional temporal processing methods to enhance predictive performance and improve model interpretability.

These strategies can be combined or applied individually depending on the specific requirements of the task and the computational resources available.

The invention further comprises a method for establishing a confidence range for test results, particularly with respect to lateral flow assays incorporating control lines. The method involves utilizing the prediction accuracy of a designated control line to determine a model confidence score, which is then used to define the confidence range for test lines. Since the control line exhibits consistent behavior across trials, deviations between the model's predicted values and expected values for the control line provide an estimate of model uncertainty. A high prediction accuracy for the control line indicates stable conditions and high model reliability, allowing for a narrower confidence range in the test lines. Conversely, greater discrepancies in the control line prediction signal higher uncertainty, necessitating a wider confidence range for the test lines. This confidence range is dynamically adjusted, ensuring that external factors such as variations in lighting conditions, image noise, or shifts in data distribution are accounted for in real-time. During training, the relationship between control line accuracy and test line confidence intervals is validated using historical data where ground truth is available, ensuring the method scales appropriately to real-world conditions. By anchoring test line confidence scores to an objective reference, this approach enhances the reliability, interpretability, and robustness of the model's predictions. In other embodiments of lateral flow assays, confidence ranges are used with two test line designs, such as a design in which one of the test lines is oriented in a competition format and the other is oriented in a sandwich format.

The method of establishing confidence ranges for test results may be further applied to the use of online learning, also referred to as adaptive learning, a machine learning methodology in which a model is continuously updated and refined based on new incoming data without requiring a complete retraining on the entire dataset. In the present invention, online learning is employed to improve model accuracy and reliability by dynamically adjusting the model's parameters based on confidence scores derived from real-time test results. When the confidence score of the control line deviates from expected values, the system can identify potential inaccuracies and update the model using newly acquired data, either through direct parameter adjustments or by selectively incorporating new labeled examples into the training set. This adaptive approach enables the model to self-correct over time, maintain robustness against variations in test conditions, and enhance predictive performance without requiring periodic offline retraining.

The invention further incorporates a method for training a machine learning model. The training process for the machine learning model involves using a sufficient number of datasets, each associated with a corresponding ground truth label, to enable the model to learn the underlying patterns necessary for accurate predictions. A portion of the training set is reserved for validation to assess model performance during training and to prevent overfitting. Prior to training, datasets undergo preprocessing through one or more of the approaches described previously. The model is trained using an optimization algorithm that iteratively adjusts its parameters to minimize a predefined loss function, which quantifies the difference between predicted and actual values. Training continues until performance metrics, such as accuracy or error reduction, reach a predefined threshold indicating sufficient learning. Once trained, the model is further evaluated on an independent test set to verify its generalization capability.

The invention further comprises a method for processing digital images using machine learning algorithms. In one embodiment of the method, the smartphone itself serves as the computational unit, executing the machine learning model directly on-device. The smartphone records the video, generates a dataset, processes the dataset using its onboard processor or dedicated AI accelerators, and presents the results to the user.

In another embodiment of the method for processing digital images using machine learning algorithms, the smartphone transfers the dataset to a connected computing device, such as a laptop or desktop computer, via a wired (USB) or wireless (Wi-Fi, Bluetooth) connection. The computing device then executes the machine learning model and returns the results to the smartphone.

In another embodiment of the method for processing digital images using machine learning algorithms, the smartphone uploads the dataset to a remote server over a network connection. The remote server, equipped with more powerful computational resources, executes the machine learning model and transmits the output back to the smartphone.

In another embodiment of the method for processing digital images using machine learning algorithms, the dataset is uploaded to a cloud-based computing infrastructure, such as an AI processing service hosted on a cloud platform. The machine learning model is executed in the cloud, and the results are returned to the smartphone.

In another embodiment of the method for processing digital images using machine learning algorithms, the system employs a hybrid approach where initial preprocessing of the dataset occurs on the smartphone, followed by the transmission of a reduced dataset to a remote computational unit (e.g., cloud or remote server) for final processing. This reduces data transfer requirements while maintaining high processing power.

In another embodiment of the method for processing digital images using machine learning algorithms, the system utilizes federated learning, where multiple smartphones locally process data and share only model updates with a central aggregation server. This approach enhances privacy by keeping raw data on the device while enabling collaborative model improvement.

In another embodiment of the method for processing digital images using machine learning algorithms, the smartphone transmits the dataset to a nearby edge computing device, such as a telecom provider's server or an AI-enabled edge node, which executes the machine learning model and returns results with minimal latency. The invention further comprises a system for analyzing the presence of one or more analytes in a sample, the system including a capillary-driven assay device designed to detect the target analyte or analytes, a digital camera configured into an assay reader, and a computing system containing a trained machine learning model, linked to the assay reader. In a preferred embodiment, a user conducts an analysis of the sample with the system using the following steps:
Collect a sample and, if required, prepare the sample for application to the assay device
Turn on the assay reader and select the procedure specific to sample/analyte of interest
As prompted by the reader, place the assay device onto the reader in its designated location
As prompted by the reader, add sample to assay device
Receive results from reader

The invention will be further described in connection with the following examples which are set forth for purposes of illustration only.

### Example 1: Digital Image Collection and Processing of a Lateral Flow Assay for Dataset Input

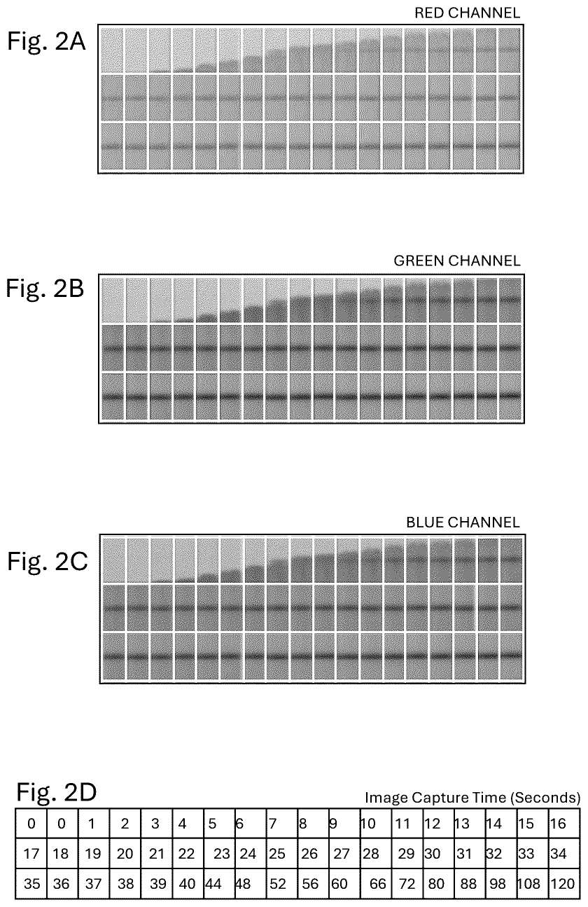

The invention comprises the collection of digital images for a reacting capillary-driven assay device. In a preferred embodiment the collection is performed on an assay reader incorporating a smartphone that automatically collects images of the device over time, beginning the collection process before sample is applied to the device and continuing the collection until the reaction is completed. Such collections may be performed with the video feature of a smartphone or collected with a customized smartphone app. Smartphone video software typically collects images at a rate ranging from 24-60 frames per second and typical capillary-driven assays have a reaction time ranging from 1-15 minutes. The input datasets used for training and inference with the invention may require considerably fewer image frames over the course of the reaction, allowing for a reduction in frames as part of the processing step. FIGS. 2A-2C show selected images for a reacting lateral flow assay collected over the course of 120 seconds on a smartphone that collected the reaction at a rate of 30 fps (thus 3,600 total frames). The processed dataset reduced the data from 3,600 to 54 images, cropping the ROI from each image and collecting these 54 cropped images into a 3×18 grid for each of the three color channels. FIG. 2D shows the respective time stamp for each of the images over the 120 second reaction time. These images collect the signal and context data with sufficient temporal resolution for analysis and reflect a minimally processed dataset suitable as input training examples for such machine learning models as convolutional neural networks (CNN) and vision transformers (ViT).

### Example 2: Representation of Signal and Context Data on a Lateral Flow Assay Image

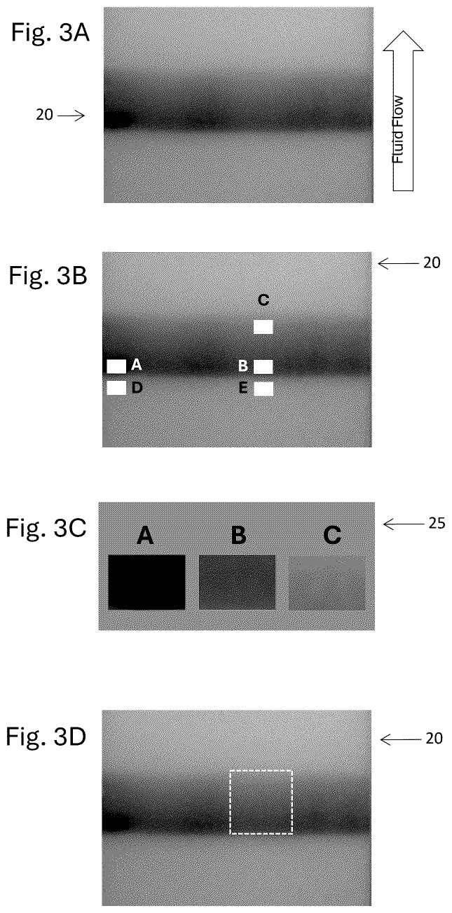

FIG. 3A shows the (green channel) digital image of test line within the ROI of a lateral flow assay device taken at the completion of a reaction 20. At magnification it can be clearly seen that the test line, measuring approximately 1×4 mm, is not a consistent or uniform collection of grayscale values across the 4 mm\hat{ }2 signal space but rather a mosaic pattern of dark and light regions. Conventional methods for analyzing this signal are to either average the grayscale values over the entire space (resulting in a significant standard deviation) or average a small section of the space (resulting in large variability depending on the location selected). Both methods limit considerably the ability to extract accurate and reliably quantitative information from the signal data. The invention described herein takes a fundamentally different approach to analyzing this signal data by granularizing the data into subsections and contextualizing each subsection to correlating context data which has also been granularized. FIG. 3B shows five subsections (approximately 60,000 microns in actual area size) of the ROI labeled A through E. Subsections A, B and C are within the signal space, allowing them to be analyzed as either signal data or context data, while Subsections D and E are outside the signal space and are therefore used only as context data. The subsections are small enough to define a uniform collection of grayscale values. Subsections A, B and C are further magnified and compared in FIG. 3C, highlighting the large difference between the three signals despite all three signals representing the same target analyte response. This difference underscores the benefits of contextualizing the various patches of signal space for improved accuracy and precision. In this example, subsection D is used for contextualizing the A signal space while subsection E is used for contextualizing the B signal space. It should be noted that essentially any context subsection can contextualize any signal subsection, particularly at higher levels of dimensionality, but the influence or weighting diminishes based on spatial or temporal distance. Thus, the assay particles flowing through the D subsection are, by and large, the same particles that will be flowing directly through the A subsection whereas the effect of the particles flowing through the E subsection would be expected to have only an indirect effect on the A subsection. Spatially located along the same flow path, the E subsection is used to also contextualize the C signal space. In this instance, the B subsection also serves as context data for analyzing the C signal space, providing information related to the conditions that define the signal. The nature of the contextualization discussed herein has primarily to do with the different densities and total number of assay particles moving through the various subsections of signal space, which will be further clarified in Examples 3 through 6.

The signal subsections described in FIGS. 3B and 3C are examples of uniform signal space. FIG. 3D shows an example of a non-uniform subsection of signal space on the same ROI. Here the signal is defined not by the mean grayscale value in the space but rather by the non-random, analyte-dependent distribution of grayscale values, presenting as a gradient of values along the direction of the flow path. There are multiple ways to define the value (and hence signal) represented in the gradient pattern such as subdividing the gradient across the flow path and expressing the subdivisions as ratios.

### Example 3: First Processed Input for Lateral Flow Assay Dataset Using Temporal Curves

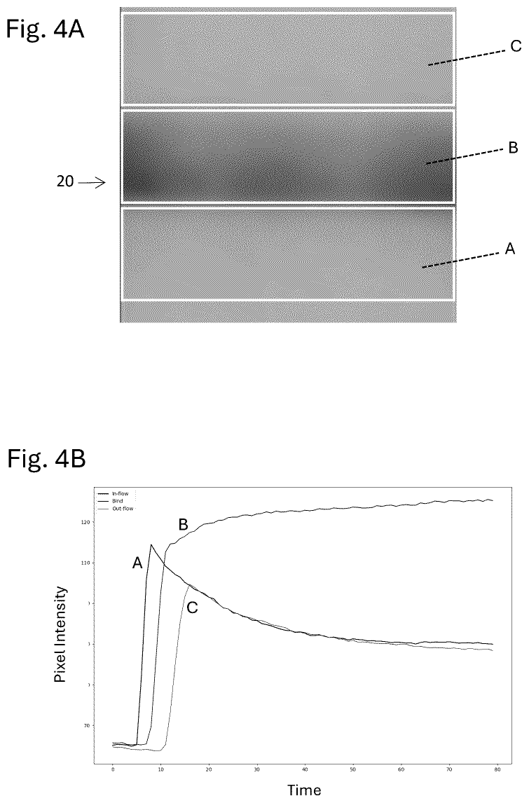

In a preferred embodiment of data representation and transformation, the invention employs an approach that may be referred to as a “temporal curve strategy” in which a grid is superimposed over the region of interest dividing the region of interest into discrete spatial units and, for each unit within the grid, a temporal curve is generated by calculating the average grayscale intensity at each time point over a sequence of digital image frames, with time represented on the x-axis and grayscale or pixel intensity on the y-axis. The resulting temporal curves capture spatial and temporal dynamics within the region of interest and these dynamics are, collectively speaking, unambiguously correlated with the target analyte concentration labels on which the models are trained. FIG. 4A shows a ROI cropped from the final image of a two-minute recording of a lateral flow assay reaction recorded on a smartphone. A 1×3 grid is superimposed over the ROI creating three discrete spatial units labeled A (an approximately 1×4 mm area immediately preceding the signal space, B (an approximately 1×4 mm area encompassing the signal space, and C (an approximately 1×4 mm area immediately following the signal space). FIG. 4B shows a graph of the temporal curves generated over the first 80 seconds of the assay reaction, plotting time on the x-axis and pixel intensity on the y-axis (pixel intensity is calculated by subtracting the mean green-channel grayscale value in the spatial unit from a white reference located on the image). The Temporal curves A and C describe analyte-independent context data essentially tracking the flow of moving reagent particles into and out of the signal space. The slope of the curves are proportional to the speed in which the particles move through the signal space and the intensity at any point on the curve is proportional to the instantaneous density of particles at that time point. Temporal curve B describes the buildup of signal over time as the particles move through and bind in the signal space. The curve indicates most of the binding occurring within the first 30 seconds of the reaction, governed by the device-specific assay conditions represented in curves A and C. In some embodiments of the temporal curve strategy, these three curves may represent the input for the reaction allowing the machine learning algorithms to learn the relationships between curve B and curves A and C or, in other words, analyze curve B “in the context” of curves A and C. For descriptive purposes, curve A may be referred to as an “inflow curve”, curve C may be referred to as an “outflow curve” and curve B may be referred to as a “bind curve”.

### Example 4: Second Processed Input for Lateral Flow Assay Dataset Using Temporal Curves

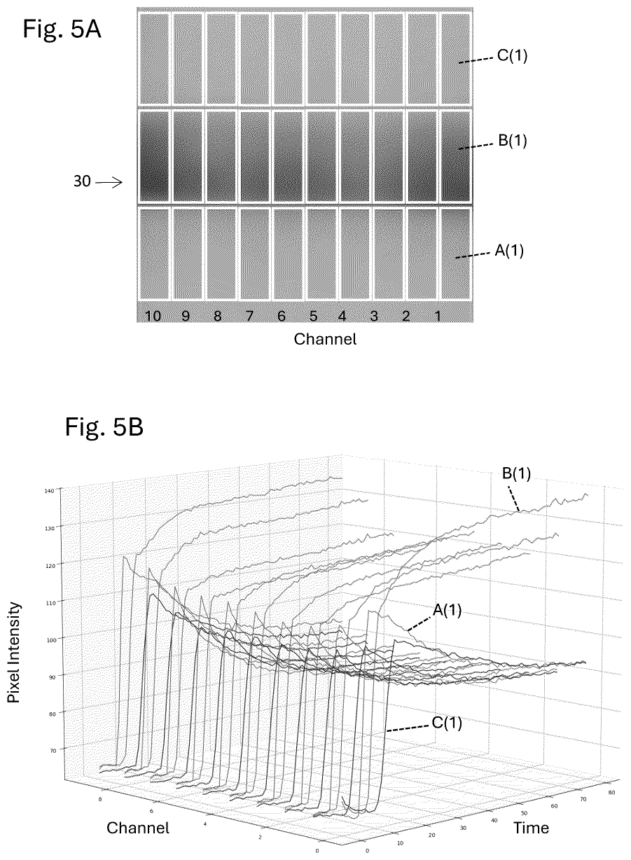

Example 3 introduced the description of temporal curves using a very “low resolution” grid that averaged the entire signal space as a single value, producing a mean result with a large standard deviation. Example 4 performs a similar analysis using a more granular grid comprised of 10×3 spatial units 30 as illustrated in FIG. 5A. This grid effectively subdivides the signal space into 10 spatial units able to generate 10 binding curves, along with a set of 10 correlating inflow/outflow curves to contextualize the binding curves. FIG. 5B shows a 3D graph of the 30 total temporal curves generated. For orientation, the spatial units outlined in channel 1 of the grid are labeled, A(1), B(1) and C(1), and the corresponding temporal curves on the FIG. 5B graph are labeled. As a further example of signal data contextualization, it can be noted that the binding curves in channels 1 and 10 are larger than the center binding curves, such as in channels 3 and 4, consistent with the overall darker appearance of these spatial units in FIG. 5A. However, the corresponding inflow/outflow curves for these binding curves reflect a similar difference. Thus, the signal value for each of the spatial units become more evenly matched when contextualized to the inflow/outflow “context data”. There are a number of ways these curves may be processed for machine learning input. A simple example would be to divide the steady-state pixel intensity of the binding curves by the average area-under-the-curve for the inflow and outflow curves, based on a baseline drawn from the steady-state of these curves (note that the steady-state of the inflow and outflow curves do not return to the initial baseline due to the fact that the hydrated nitrocellulose membrane will show a darker color relative to the dry membrane irrespective of particle density).

### Example 5: Third Processed Input for Lateral Flow Assay Dataset Using Temporal Curves

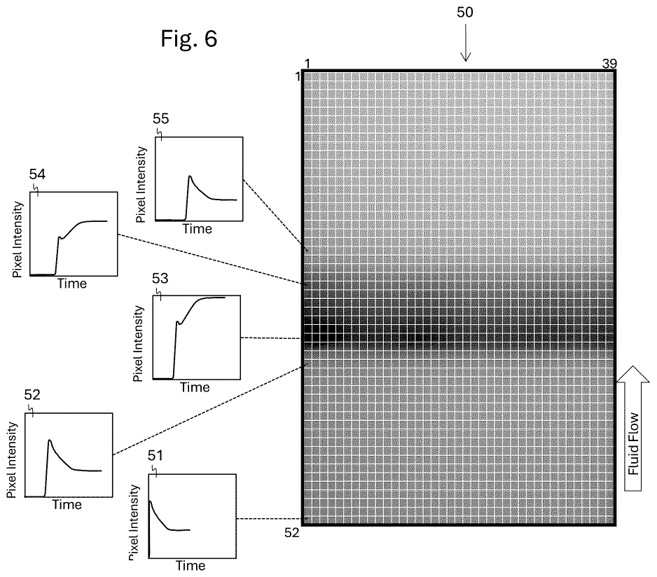

Example 4 demonstrates an improvement in data analysis compared with Example 3 by increasing the grid number. This strategy may be continued for further improvement. FIG. 6 shows greater granularization of the ROI using a 39×52 grid of spatial units 50. This grid subdivides the signal space into 390 uniform units with each signal unit having access to as many as 2,027 context units. Illustration of all the temporal curves in the FIG. 6 example is impractical, thus a small selection of five curves, all residing in channel 1, is shown for discussion. The first curve to form is the inflow curve 51 shown at the bottom of the grid followed by inflow curve 52 forming in a unit near the signal space. Comparing the two curves, the curve 51 is closer to the y-axis, owing to its earlier formation, and has a steeper slope after its peak, due to the assay particles moving faster at this earlier position. While either curve alone may provide context information for this section of the ROI, multiple curves may substantially increase the usability of the context data. Also, in some embodiments, temporal curve 51 and similar curves in this portion of the ROI may serve as “start signals” indicating the time at which the other curves downstream begin. The next two curves shown are bind curves, 53 and 54, located in the signal space. Curve 53 shows a stronger signal compared with 54 and may also serve as context data for 54. The remaining curve, 55, is an outflow curve exhibiting the farthest position away from the y-axis reflecting it to be the last curve in these examples to be generated, the smallest peak compared with the inflow curves reflecting the fewest assay particles flowing through this section, and the flattest slope reflecting the slowest movement of particles. The use of a large number of temporal curves, along with each curve providing information related to multiple variables of the assay condition, may be said to constitute a “high dimensional contextualization of the signal data” and is suitable for deep learning applications and related strategies, such as tokenization of the curves.

### Example 6: Processed Input Dataset Using Spatial Signal Gradients

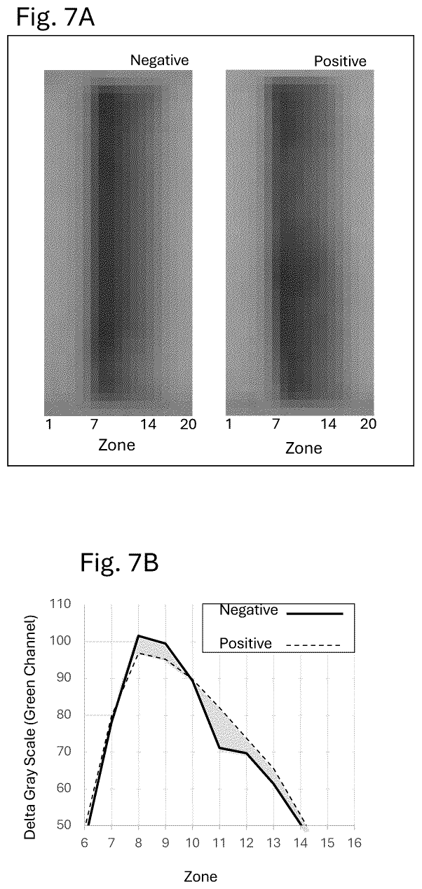

In addition to analyzing signal data as uniform patches of grayscale values as described in Example 6, the invention takes advantage of the surprising finding that the signal space of lateral flow assays are also capable of providing analyte-dependent data in the form of color gradient formations, the gradient forming perpendicular to the fluid flow and most prominent in the portion of the dynamic range with the highest amount of binding, such as around the detection limit of a competitive assay. FIGS. 7A and 7B provide an example of this phenomenon. FIG. 7A shows the digital image of a pair of lateral flow test lines from competitive lateral flow assay devices designed to detect amphetamine qualitatively at a cutoff of 500 ng/ml. The image on the left shows a test line from an assay device reacted with negative sample. The image on the right shows a test line from an assay device reacted with positive sample at a concentration of 20 ng/ml, a concentration that would not be expected to have any effect on the test line and, in the case of these two test, appears to have no impact in terms of mean grayscale values. However, if the test lines are subdivided into 9 zones and the mean grayscale values of each individual zone are plotted to express the color gradient numerically, a difference appears between the test lines. FIG. 7B is a graph showing this difference. The negative sample (solid line) exhibits darker zones toward the leading edge of the test line (zones 8 and 9) while the positive sample (dotted line) exhibits darker zones toward the back end of the test line (zones 11, 12 and 13). This gradient effect may be used to define a secondary signal value in the signal space, such as a value defined by the ratio of the front zones divided by the back zones or the ratio of the darkest zone divided by the average of all zones.

### Example 7: Signal and Context Data from a Dry-Reagent Chemistry Assay Device Digital Image

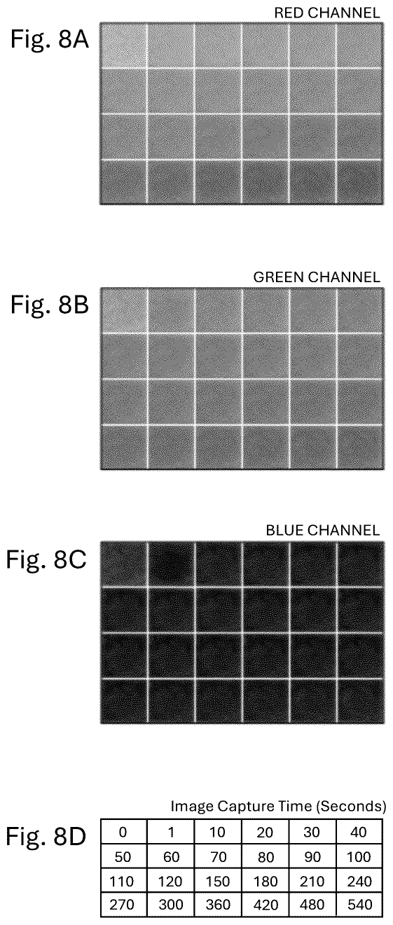

The invention comprises the collection of digital images for a reacting capillary-driven assay device. In a preferred embodiment the collection is performed on an assay reader incorporating a smartphone that automatically collects images of the device over time, beginning the collection process before sample is applied to the device and continuing the collection until the reaction is completed. Such collections may be performed with the video feature of a smartphone or collected with a customized smartphone app. Smartphone video software typically collects images at a rate ranging from 24-60 frames per second and typical capillary-driven assays have a reaction time ranging from 1-15 minutes. The input datasets used for training and inference with the invention may require considerably fewer image frames over the course of the reaction, allowing for a reduction in frames as part of the processing step. FIGS. 8A-8C show selected images for a reacting dry reagent chemistry assay collected over the course of 540 seconds on a smartphone that collected the reaction at a rate that ranged from 1 frame per second to 1 frame per 60 seconds, cropping the ROI from each image and collecting these 24 cropped images into a 4×6 grid for each of the three color channels. FIG. 8D shows the respective time stamp for each of the images over the 540 second reaction time. These images collect the signal and context data with sufficient temporal resolution for analysis and reflect a minimally processed dataset suitable as input training examples for such machine learning models as convolutional neural networks (CNN) and vision transformers (ViT).

### Example 8: Processed Input Dataset for a Dry-Reagent Chemistry Assay Device

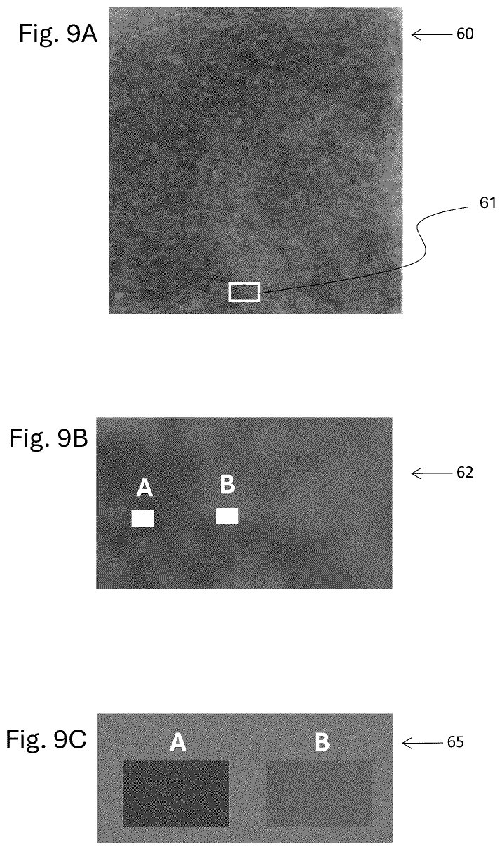

FIG. 9A shows the magnified (green channel) digital image of a reaction pad within the ROI of a dry reagent chemistry assay device taken at the completion of a reaction 60. At magnification it can be clearly seen that the test pad, measuring approximately 4×4 mm, is not a consistent or uniform collection of grayscale values across the 16 mm\hat{ }2 signal space but rather a mosaic pattern of dark and light regions. Conventional methods for analyzing this signal are to either average the grayscale values over the entire space (resulting in a significant standard deviation) or average a small section of the space (resulting in large variability depending on the location selected). Both methods limit considerably the ability to extract accurate and reliably quantitative information from the signal data. The invention described herein takes a fundamentally different approach to analyzing this signal data by granularizing the data into subsections and contextualizing each subsection to correlating context data which has also been granularized. FIG. 9B shows a magnified subsection of the FIG. 9A reaction pad 62, outlined on the pad as a white rectangle 61. Two further subsections of the pad, labeled A and B, are shown excised out of the image in FIG. 9B and further magnified in FIG. 9C, 65. The subsections are small enough to define a uniform collection of grayscale values and exhibit clearly different mean values despite representing the same target analyte response. In this example, subsection B may be treated as context data when considering subsection A. For example, the model may compare the temporal value of B to A to determine if the region of B is lighter because it saturated with sample at a later timepoint compared with A. If B exhibits a uniform temporal value over the same period of time wherein A shows a non-uniform value progressing to a darker color, the model may contextualize this as a border between two discrete reaction patches within the pad, temporally separated. If, on the other hand, B exhibits a non-uniform temporal value over the same period of time as A, the model may interpret this as a reagent displacement phenomenon and analyze the A subregion in the context of having a higher-than-average concentration of reagent reacting in the signal space. In this sense, A may also be analyzed as context data allowing the model to interpret the signal value of B in the context of having a lower-than-average concentration of reagent reacting in the signal space. In another embodiment, the model may interpret the larger subsection 62 as a non-uniform signal in which two different signal values may be averaged, possibly with different weightings.

### Example 9: Processed Input for Dry-Reagent Chemistry Dataset Using Temporal Curves

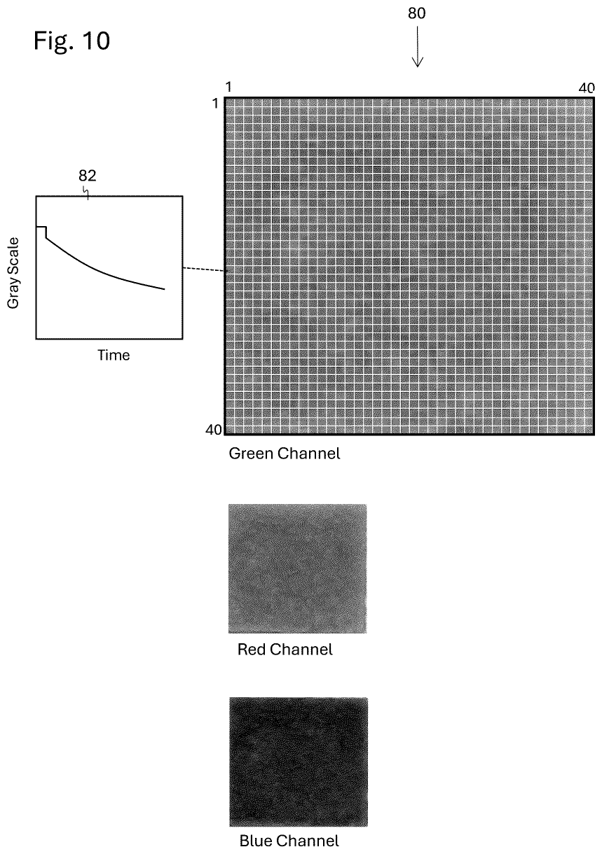

Unlike lateral flow assays that typically rely on reagent particles having one stable color hue that allows for analysis in a single color channel, dry-reagent chemistry assays often rely on a change in color hue requiring the use of all three color channels. With three channels, the reaction may be analyzed not simply by the change in grayscale value for any one channel but also by the relationship between channels. FIG. 10 shows the signal space for the ROI of a dry-reagent chemistry reaction pad for the three color channels, wherein the green channel space 80 has been magnified and subdivided by a 40×40 grid generating 1600 spatial units. For each spatial unit, a temporal curve is measured (thus 4,800 total temporal curves for all three channels in this example). The measurement commences when the user is prompted to add sample to the assay device. Illustration of all the temporal curves in the FIG. 10 example is impractical, thus only one representative curve 82 is shown for discussion. Characteristic of this curve is the temporally uniform signal (flat horizontal line) at the beginning of the curve. The change in this uniform signal effectively indicates the “start time” for the reaction within the particular subregion of the signal space and allows all 1,600 subregions to be assessed independently with respect to start time. Each subregion may then be contextualized to the other subregions in the ROI as described in Example 8.

### Example 10: Customized First Design of a Lateral Flow Assay Device

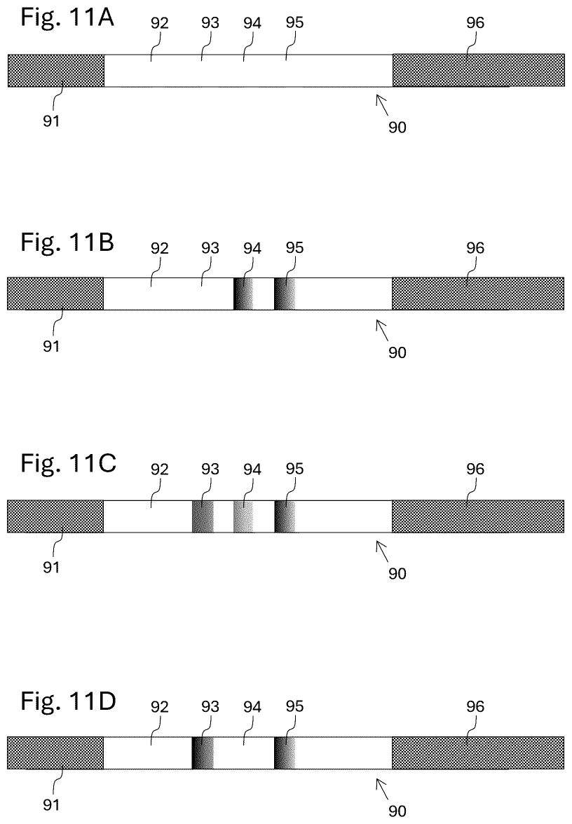

The invention accommodates two features that may be further enhanced by the customized design of lateral flow assay devices. The first feature, noted in Example 6, is the surprising finding that signal patterns, primarily in the form of gradients along the width of test lines, correlate with analyte concentration, particularly at the low end of concentration. The second feature, noted in the above text, is that the invention comprises novel methods for self-supervised and online/adaptive learning which may be greatly amplified by analyte-independent patterns in the reaction, allowing for continued training with unknown, and hence unlabeled, sample data such as assays run “in-the-field” on commercial applications of the invention. Additionally, the design accommodates a novel approach for assigning quality control “confidence scores” to the strips. FIGS. 11A through 11D are diagrammatic top views of a lateral flow assay strip design 90 customized to accommodate these features. The basic strip architecture is comprised of a sample addition component 91, membrane supporting the assay reactions 92, sample waste component 96, and backing support (bottom side, not shown). On the membrane are a control zone 95, a test zone configured for direct, sandwich binding of analyte 93, and a test zone configured for competitive binding of analyte 94. The binding areas of the test and control zones are designed with wider widths than the convention narrow lines of typical lateral flow assay devices, and as such will be referred to herein as “bands” rather than lines. FIG. 11A shows the strip before sample is applied. FIG. 11B shows the strip following the addition of negative sample. In the absence of analyte, no band forms in the sandwich zone whereas a maximum possible band forms in the competitive zone along with a corresponding band forming in the control zone. FIG. 11C shows the strip following the addition of positive sample at a concentration in the middle of the assay's dynamic range, resulting in the formation of two test bands and a corresponding control band. FIG. 11D shows the strip following the addition of positive sample at a concentration above the upper limit of the assay's dynamic range. At this level, no band forms in the competitive zone whereas a maximum possible band forms in the sandwich zone and a corresponding band forms in the control zone. The novel use of these three bands for self-supervised and online learning will be described further in Example 13.

### Example 11: Customized Second Design of a Lateral Flow Assay Device

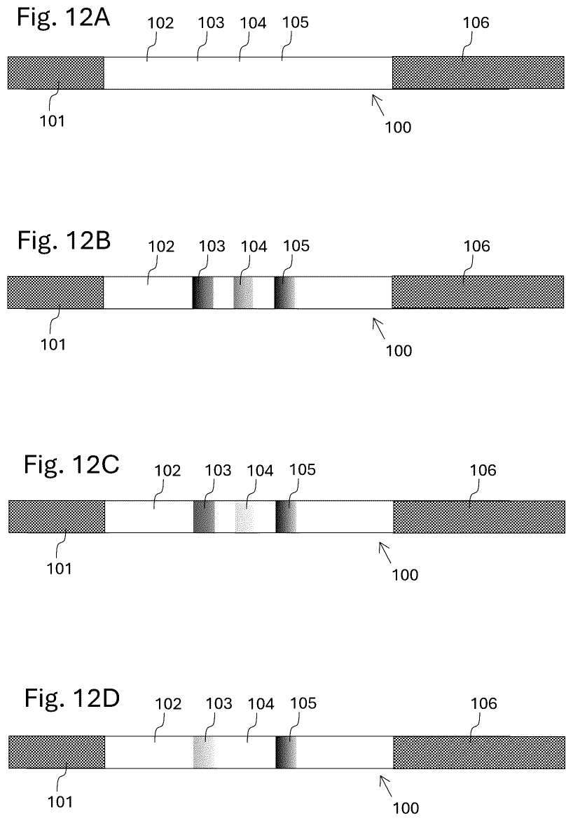

Example 10 describes a customized design of a lateral flow assay device capable of accommodating a sandwich binding zone. For small molecule analytes, the sandwich orientation is generally not an option. The customized second design of a lateral flow device is meant to accommodate these small molecule analytes and is comprised of two competitive binding zones prepared at two different concentrations of capture reagent. FIGS. 12A through 12D are diagrammatic top views of a lateral flow assay strip design 100 customized to accommodate these features. The basic strip architecture is comprised of a sample addition component 101, membrane supporting the assay reactions 102, sample waste component 106, and backing support (bottom side, not shown). On the membrane are a control zone 105, a test zone configured for competitive binding of analyte at a high level of particle conjugate binding (low analyte sensitivity) 103, and a test zone configured for competitive binding of analyte at a low level of particle conjugate binding (high analyte sensitivity) 104. The binding areas of the test and control zones are designed with wider widths than the convention narrow lines of typical lateral flow assay devices, and as such will be referred to herein as “bands” rather than lines. FIG. 12A shows the strip before sample is applied. FIG. 12B shows the strip following the addition of negative sample. In the absence of analyte, a maximum possible band forms in both competitive zones along with a corresponding band forming in the control zone. The low sensitivity band exhibits a darker color (higher pixel intensity) compared with the high sensitivity band. FIG. 12C shows the strip following the addition of positive sample at a concentration in the middle of the assay's dynamic range, resulting in the formation of two test bands of lighter color compared with the negative sample. FIG. 11D shows the strip following the addition of positive sample at a sufficiently high enough concentration to completely eliminate the formation of a test band in the high sensitivity test zone. The novel use of these three bands for self-supervised and online learning will be described further in Example 13.

### Example 12: Customized Third Design of a Lateral Flow Assay Device (Inferred Flow Pattern)

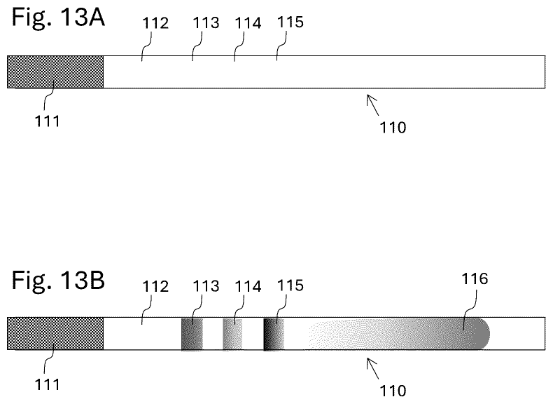

While traditional lateral flow assays do not incorporate the measurement of the reaction flow stream during the course of the assay reaction, this measurement is a fundamental step in the current invention. Preferred embodiments of this measurement comprise the recording of the flow movement over the course of the reaction using digital video. In another preferred embodiment, the flow stream may be inferred from the spatial pattern downstream of the test and control lines/bands using fewer images, such as one or two images, at a designated time point or time points. FIGS. 13A and 13B illustrate an example of this embodiment in a customized lateral flow assay device. The device 110 is designed similarly to the Example 10 device with the exception of the sample waste component being removed and the nitrocellulose membrane 112 being extended the length of the strip. FIG. 13A shows the device, prior to the addition of sample, comprising a sample addition component 111, test binding areas 113, 114 and a control binding area 115. FIG. 13B shows the device following the addition of sample and after a sufficient period of time for the three bands to form. At this point the flow stream of unbound particles has migrated sufficiently beyond the bands and is visible as a color pattern 115 toward the terminal end of the device. While the flow stream is not directly measured, the movement of the particles up to and through the three bands may be sufficiently inferred from the pattern with a sufficient number of training examples.

### Example 13: Prediction of Control and Test Bands for Confidence Score and Online Training

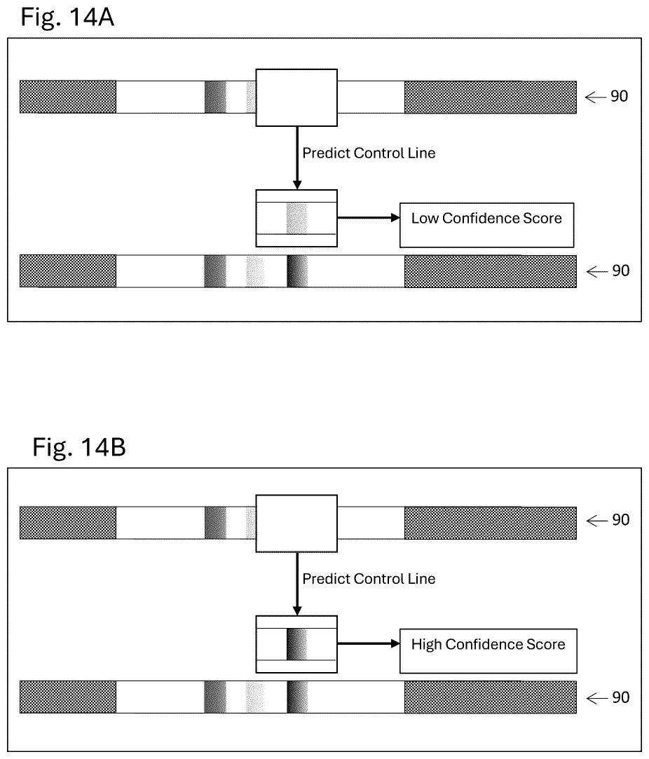

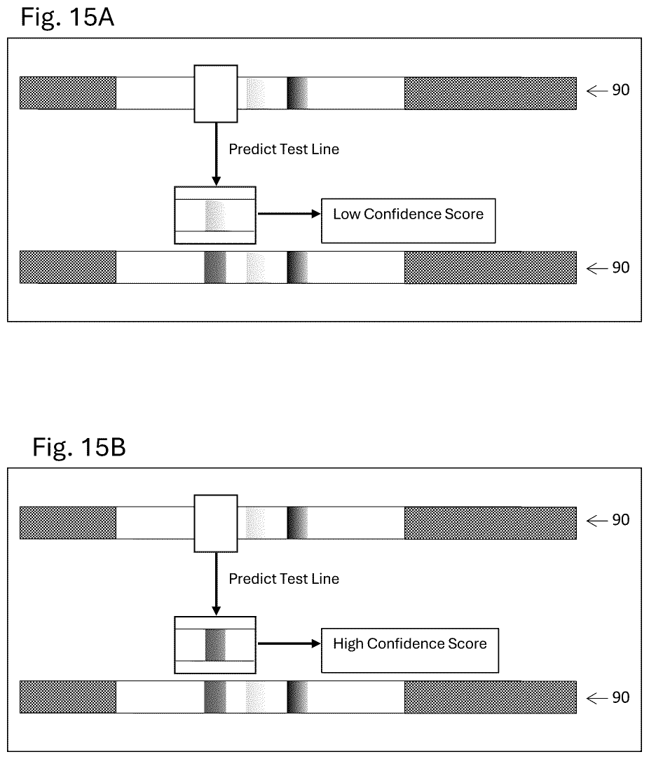

The invention comprises a method for self-supervised learning, and for establishing a confidence range for test results, particularly with respect to lateral flow assays incorporating control lines and customized lateral flow assays such as the assays described in Examples 10 through 12. The method involves utilizing the prediction accuracy of a designated control line/band, or one of the two test lines/bands in the customized assays, to determine a model confidence score. This method of establishing confidence ranges may be further applied to the use of online learning, also referred to as adaptive learning, a machine learning methodology in which a model is continuously updated and refined based on new incoming data without requiring a complete retraining on the entire dataset. In the invention, online learning is employed to improve model accuracy and reliability by dynamically adjusting the model's parameters based on confidence scores derived from real-time test results. FIGS. 14A, 14B, 15A and 15B provide a visual illustration of how this process may occur on a lateral flow assay device 90 such as the device described in Example 10. FIG. 14A illustrates a scenario where the control band is “hidden” from the model and the model is then tasked with predicting the appearance of this band using the assay reaction data (which includes flow stream data not shown in the figure). In this first scenario the model's prediction is considerably different from the actual control band color intensity and it is consequently assigned a low confidence score. In contrast, the prediction that the model makes in FIG. 14B very closely matches the actual color intensity of the control band and is thus given a high confidence score. A similar approach is taken in FIGS. 15A and 15B with respect to the first test band, hidden from the model which is then challenged with predicting the appearance based on the appearance of the second test band along with the flow stream data (in some embodiments, the control band may also be considered).

### Example 14: Demonstration of Improved Sensitivity of Lateral Flow Assay Device

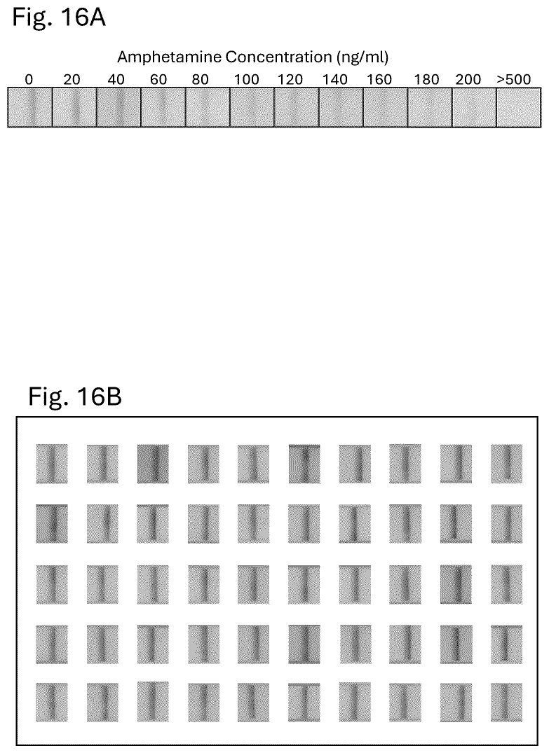

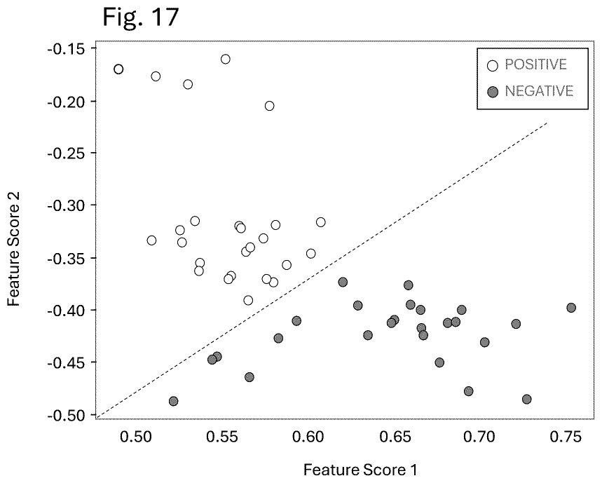

The invention is designed to improve the sensitivity of capillary-driven assays such as lateral flow assays. To demonstrate this ability, a study was conducted with a set of commercial lateral flow immunoassay devices designed to detect the drug amphetamine at a qualitative cutoff of 500 ng/ml amphetamine. The aim of the study was to show that the invention could significantly lower the sensitivity of this assay far below the cutoff for which it was designed. The first part of the study was to determine the lowest concentration of amphetamine for which the positive sample could not be distinguished from negative results by any currently available methods. Amphetamine was spiked into a synthetic urine matrix at serially diluted levels between 0 and 200 ng/ml. These samples were then tested on the amphetamine assay devices and a concentration was selected based on the level at which the positive sample was indistinguishable from the negative sample based on either visual observation or measuring the average grayscale value of the test lines with ImageJ software. From this study it was determined that 20 ng/ml was indistinguishable from 0 ng/ml using the criteria described. FIG. 16A shows a representative set of test line results from assay devices tested at the levels shown. A machine learning model was then trained on approximately 200 results of these devices using labeled results of assay devices such as the ones shown in FIG. 16A. The model used a combination of strategies including random forest analysis, and data representation and transformation was based on a temporal curve strategy using figures similar to the curves shown in FIG. 5B. The model was trained on amphetamine concentrations ranging from 0 to 500 ng/ml. A second form of feature extraction was established based on the gradient signal observations described in Example 6. The feature defined a gradient value by dividing the test line into zones, such as the zones described in Example 6, and defining a value based on the pixel intensity of the leading edge zones divided by the overall test line intensity. A study was then conducted to determine how well these two features (temporal curve contextualization and gradient signal contextualization) could distinguish positive and negative results. FIG. 16B shows the digital images of test lines for 50 samples, 25 negative and 25 positive (20 ng/ml amphetamine). The images are placed in random order and clearly show that the positive and negative results cannot be visually discerned. Remarkably, the invention was able to distinguish between the two sets with 100% sensitivity and 100% specificity. FIG. 17 shows a scatterplot of the 50 samples in which two “Feature Scores” are plotted. Feature Score 1 is based on the temporal curve contextualization and Feature Score 2 is based on the gradient signal contextualization. From this plot a clear straight line is easily drawn to separate the positive results from the negative results. All video datasets used in this study were collected on an iPhone® SE.

### Example 15: Vertical Design of Assay Reader and Capillary-Driven Assay Device

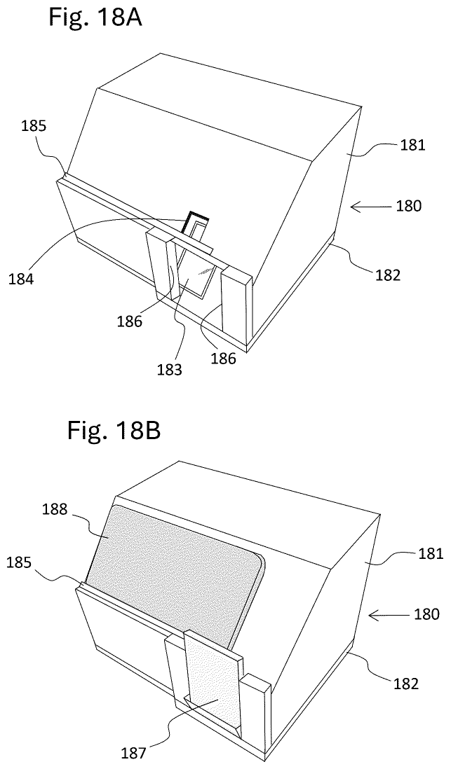

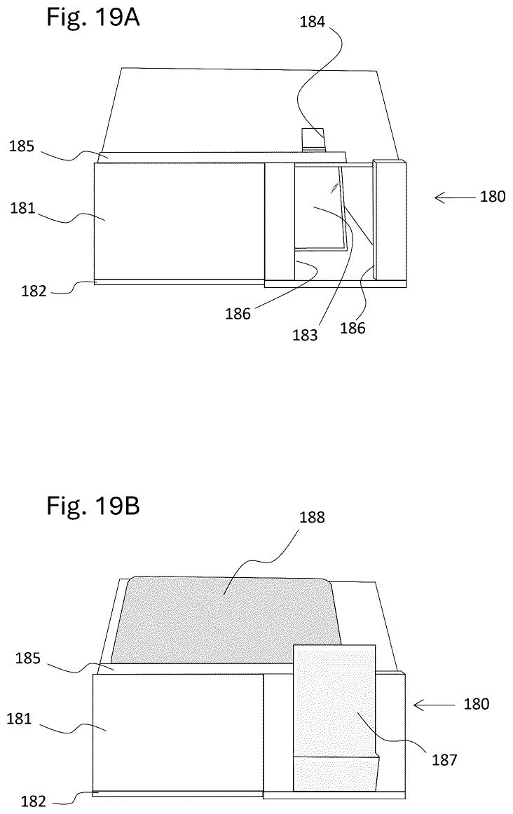

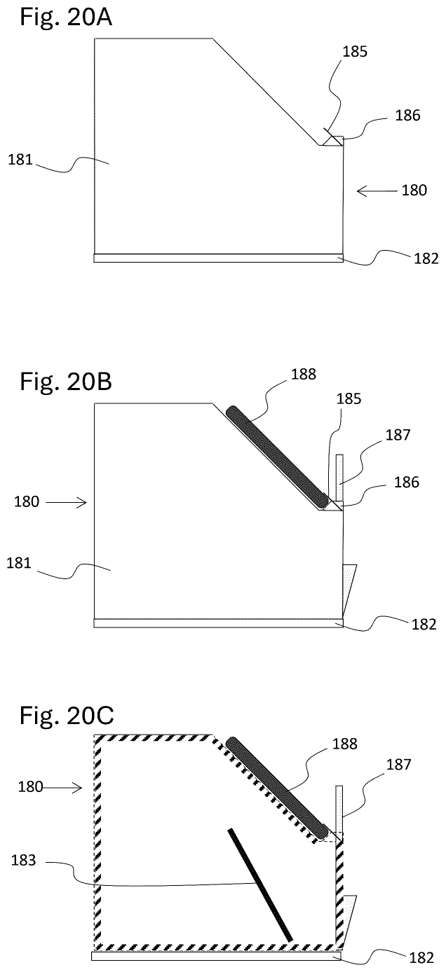

The invention incorporates digital cameras to capture one or more digital images of capillary-driven assays over the course of an assay reaction, capturing within these images a dataset processable into signal data and context data. The digital camera is configured into an assay reader designed to efficiently collect digital images from a reacted capillary-driven assay. In a preferred embodiment, the digital camera incorporated in the invention is supplied by a smartphone. FIG. 18A depicts the perspective view of an embodiment of an assay reader housing 180 designed to incorporate a smartphone. The housing comprises a base 182 and a shell 181. The shell contains a rack 185 for holding a smartphone on the outer surface of the shell and an opening 184 allowing the camera lens to record images inside the housing and the camera light to illuminate inside the housing. The shell also contains a device holder 186 designed for holding a capillary-driven assay device in a vertical orientation. Inside the housing is a mirror 183 positioned in such a way as to allow the camera to record the assay reaction on the device through the reflection of the mirror. The mirror may be attached to either the base or the shell and may be fixed or adjustable with respect to its angle of orientation. The housing may be optimized to allow for even illumination of light onto the device, such as by placing diffusion material over the light and painting the inner surface of the shell and base a matte white color. FIG. 18B shows a perspective view of the assay reader housing with a smartphone 188 and an assay device 187 placed within the designated positions on the shell. FIG. 19A shows a diagrammatic front view of the FIG. 18A assay housing and FIG. 19B shows a diagrammatic front view of the FIG. 18B housing with a smartphone and assay device in their designated locations, creating the assay reader. FIG. 20A shows a diagrammatic side view of the FIG. 18A assay housing and FIG. 20B shows a diagrammatic side view of the FIG. 18B housing with a smartphone and assay device in their designated locations, creating the assay reader. From the side view, it can be noted that the smartphone is positioned on the reader at an angle of about 45° relative to the base. FIG. 20C depicts a sectional view of the FIG. 20B assay reader exposing the mirror inside the reader. The mirror is positioned and angled relative to both the smartphone camera lens and the reacting surface of the assay device in such a way as to allow the camera to sufficiently record the reaction occurring on the device through the reflection in the mirror.

### Example 16: Horizontal Design of Assay Reader and Capillary-Driven Assay Device

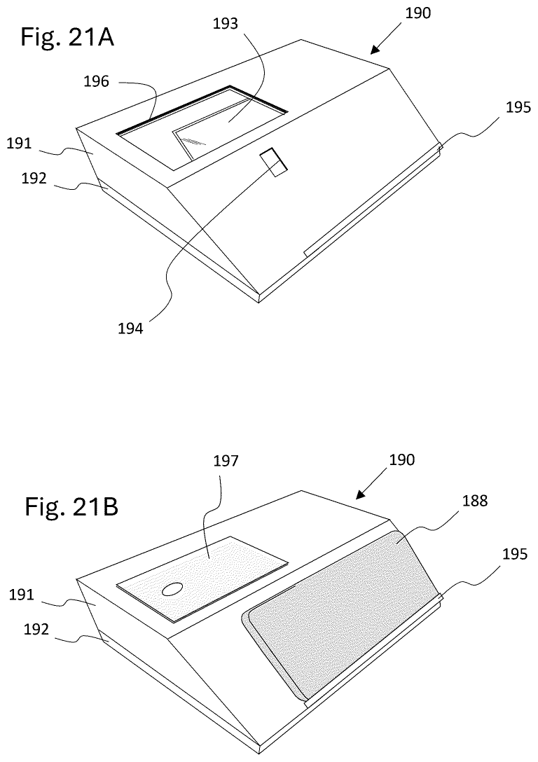

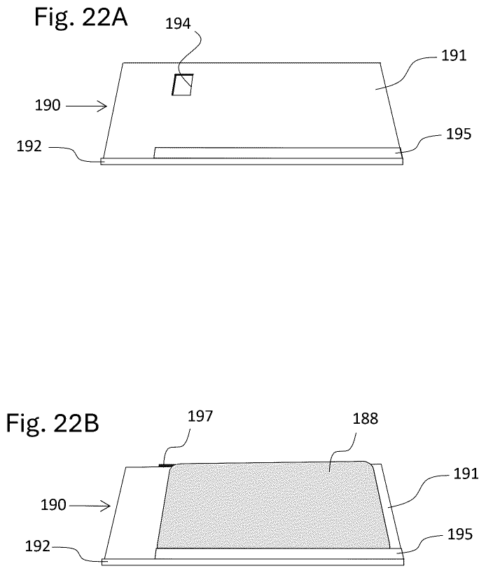

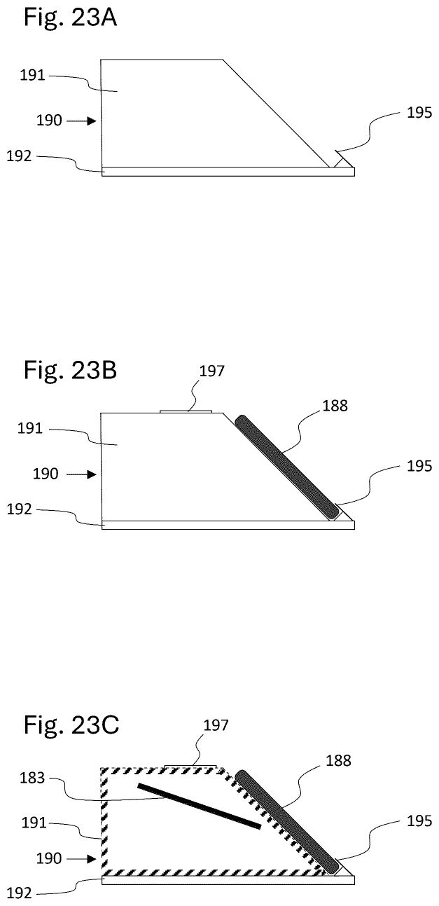

In a second preferred embodiment of an assay reader, the reader is designed to position the capillary-driven assay device in a horizontal orientation relative to the base. FIG. 21A depicts the perspective view of an embodiment of an assay reader housing 190 designed to incorporate a smartphone. The housing comprises a base 192 and a shell 191. The shell contains a rack 195 for holding a smartphone on the outer surface of the shell and an opening 194 allowing the camera lens to record images inside the housing and the camera light to illuminate inside the housing. The shell also contains a device holder 196 designed for holding a capillary-driven assay device in a horizontal orientation. Inside the housing is a mirror 193 positioned in such a way as to allow the camera to record the assay reaction on the device through the reflection of the mirror. The mirror may be attached to either the base or the shell and may be fixed or adjustable with respect to its angle of orientation. The housing may be optimized to allow for even illumination of light onto the device, such as by placing diffusion material over the light and painting the inner surface of the shell and base a matte white color. FIG. 21B shows a perspective view of the assay reader housing with a smartphone 188 and an assay device 197 placed within the designated positions on the shell. FIG. 22A shows a diagrammatic front view of the FIG. 21A assay housing and FIG. 22B shows a diagrammatic front view of the FIG. 21B housing with a smartphone and assay device in their designated locations, creating the assay reader. FIG. 23A shows a diagrammatic side view of the FIG. 21A assay housing and FIG. 23B shows a diagrammatic side view of the FIG. 21B housing with a smartphone and assay device in their designated locations, creating the assay reader. From the side view, it can be noted that the smartphone is positioned on the reader at an angle of about 45° relative to the base. FIG. 23C depicts a sectional view of the FIG. 23B assay reader exposing the mirror inside the reader. The mirror is positioned and angled relative to both the smartphone camera lens and the reacting surface of the assay device in such a way as to allow the camera to sufficiently record the reaction occurring on the device through the reflection in the mirror.

### Example 17: Customized Assay Device in Vertical Orientation: Lateral Flow Format

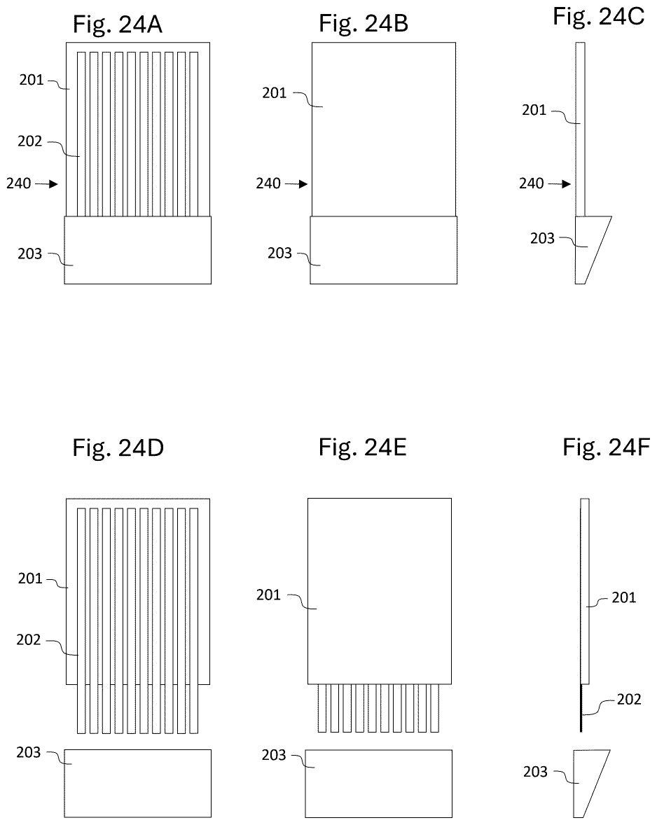

The device 187 introduced in FIG. 18B is designed to incorporate any capillary-driven assay capable of running in a vertical orientation. FIG. 24A through 24F shows a set of diagrammatic drawings describing the incorporation of lateral flow assay strips onto the device. FIG. 24A shows a diagrammatic front view of an assay device 240 comprising a backing 201 and a trough cap 203. Attached to the backing are a set of lateral flow strips 202 oriented to run vertically with fluid flow moving from the bottom upward via capillary action. The front view is faced inward on the reader so that the strips may be recorded by the digital camera. FIG. 24B shows the back view of the FIG. 24A device and FIG. 24C shows a side view of the FIG. 24A device. From the side view the opening of the trough cap is visible. This opening allows for the addition of sample onto the device while the device is positioned on the assay reader. FIG. 24D is a diagrammatic front view of the FIG. 24A device with the trough cap detached from the backing, allowing for a view of the portions of the lateral view strips which extend into the trough cap. FIG. 24E is a diagrammatic back view of the FIG. 24D device components and FIG. 24F is a diagrammatic side view of the FIG. 24D components.

### Example 18: Customized Assay Device in Vertical Orientation: Dry-Reagent Chemistry Format

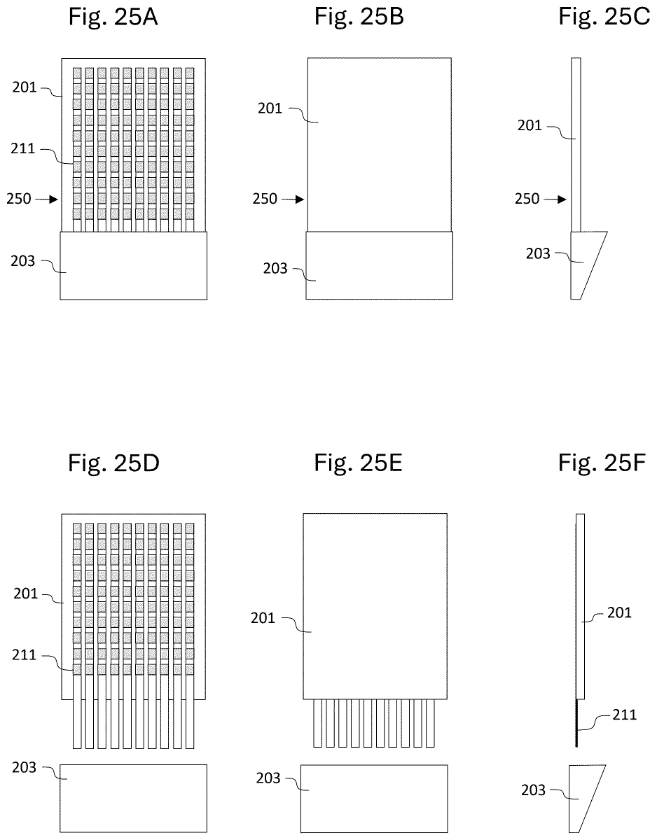

FIG. 25A through 25F shows a set of diagrammatic drawings describing the incorporation of dry-reagent chemistry assay strips onto the device. FIG. 25A shows a diagrammatic front view of an assay device 250 comprising a backing 201 and a trough cap 203. Attached to the backing are a set of ten dry-reagent chemistry strips 211 oriented to run vertically with fluid flow moving from the bottom upward via capillary action. Each of the strips on the device contains ten separate assay reaction pads, thus the device is able to measure up to 100 unique analytes in a single sample. The front view is faced inward on the reader so that the pads may be recorded by the digital camera. FIG. 25B shows the backview of the FIG. 25A device and FIG. 25C shows a side view of the FIG. 25A device. From the side view the opening of the trough cap is visible. This opening allows for the addition of sample onto the device while the device is positioned on the assay reader. FIG. 25D is a diagrammatic front view of the FIG. 25A device with the trough cap detached from the backing, allowing for a view of the portions of the strips which extend into the trough cap. FIG. 25E is a diagrammatic back view of the FIG. 25D device components and FIG. 25F is a diagrammatic side view of the FIG. 25D components.

### Example 19: Testing a Sample on a Vertically Oriented Assay Reader

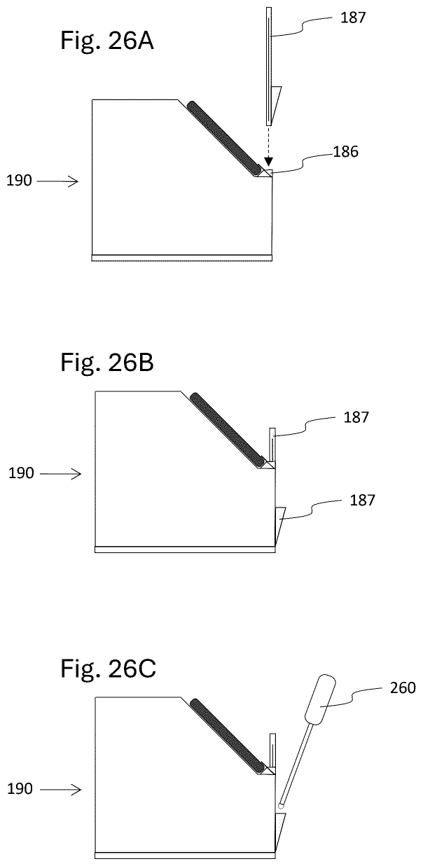

FIGS. 26A through 26C are diagrammatic side views demonstrating the basic steps of testing a sample on the FIG. 18B assay reader using an assay device such as the devices described in FIGS. 24 and 25. FIG. 26A depicts a device 187 being inserted vertically into the into the device holder 186 of the assay reader 190. FIG. 26B shows the device once it has been fully inserted into the reader. In a preferred embodiment, the user engages the smartphone screen to indicate that the device has been inserted. In other embodiments, the reader incorporates a sensor which automatically informs the smartphone a device has been inserted. Once the smartphone is engaged, it will prompt the user on the appropriate time to add sample. In a preferred embodiment, the smartphone will begin collecting images of the device before the user is prompted to add sample. FIG. 26C depicts the sample being added into the device trough with a sample pipet 260.

### Example 20: Recording an Assay Reaction while Sample is Applied to a Vertically Oriented Assay Device on an Assay Reader

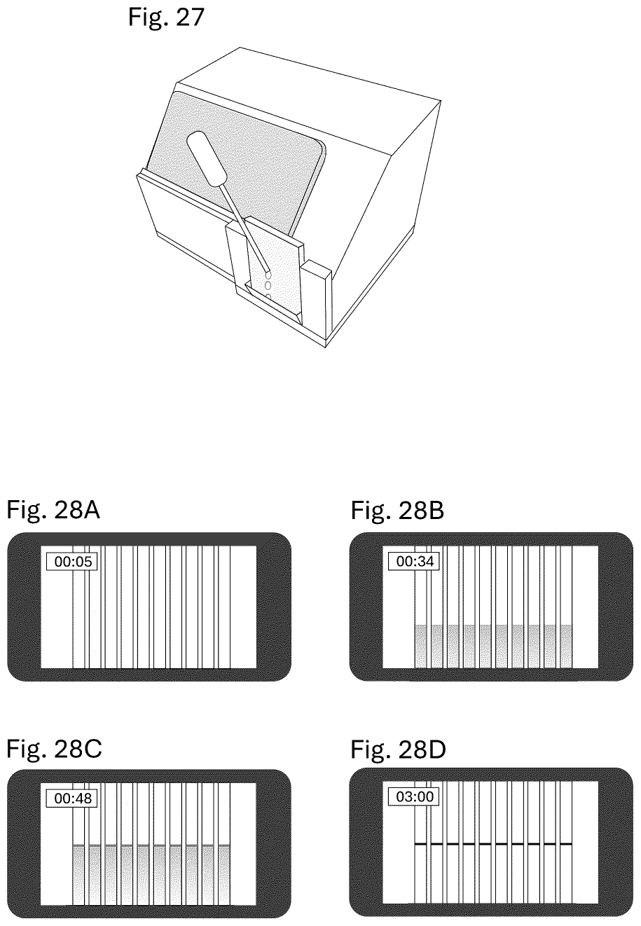

FIG. 27 shows the perspective view of the FIG. 26C assay reader with sample being added to the device trough. As noted in the previous example, the smartphone begins recording images of the device prior to addition of the sample, ensuring the capture of the entire assay reaction from initiation. FIG. 28A through 28D depicts the capture of assay reaction images for a lateral flow based device such as the device described in FIG. 24. The images depict the smartphone screen of the assay reader at four different time points in a video image recording. FIG. 28A shows the ten lateral flow strips on the assay device being recorded 5 seconds after prompting the user to add sample. At this early time point, the fluid sample has not yet reached the recorded section of the strips. FIG. 2 shows the strips 34 seconds after prompting, at which point a flow stream pattern is evident on all ten strips. FIG. 28C shows the strips 48 seconds after prompting, at which point the flow stream is seen reaching the test line region for the strips. FIG. 28D shows the strips 3 minutes after prompting, at which point the reaction is essentially completed for all ten strips and a test line is formed on all ten strips (note that the visualization of the reaction on the smartphone screen is shown to describe the recording process in this example and is not a necessary requirement for the invention).

The easy access to the device trough for sample addition, while simultaneously viewing the prompting on the smartphone screen, underscores the benefits of the assay reader design with the incorporated mirror, enabling both the screen and trough to be placed on the same user-facing side of the instrument.

### Example 21: Customized Assay Device in Horizontal Orientation: Lateral Flow Format

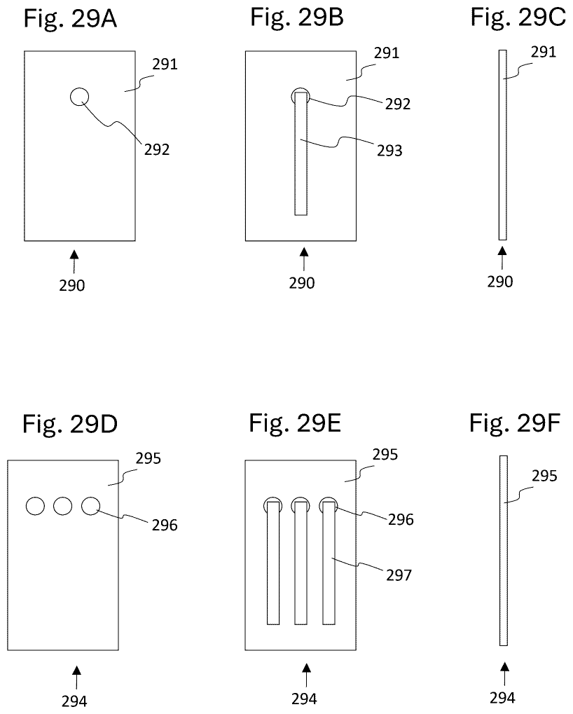

The device 197 introduced in FIG. 21B is designed to incorporate any capillary-driven assay capable of running in a horizontal orientation. FIGS. 29A through 29F show two sets of diagrammatic drawings describing the incorporation of lateral flow assay strips onto the device. FIG. 29A shows a first embodiment of a diagrammatic top view of an assay device 290 comprising a backing 291 and a sample application port 292. FIG. 29B shows the diagrammatic bottom view of the FIG. 29A device where a lateral flow assay strip 293 is attached and viewable by an assay reader camera. FIG. 29C is a diagrammatic side view of the FIG. 29A device. FIG. 29D is a second embodiment of a diagrammatic top view of an assay device 294 comprising multiple lateral flow assay strips on a single device. The device comprises a backing 295 and multiple sample ports 296. FIG. 29D shows the diagrammatic bottom view of the FIG. 29C device where a lateral flow assay strips 297 are attached and viewable by an assay reader camera. FIG. 29F is a diagrammatic side view of the FIG. 29D device.

### Example 22: Customized Assay Device in Horizontal Orientation: Dry-Chemistry Reagent Format

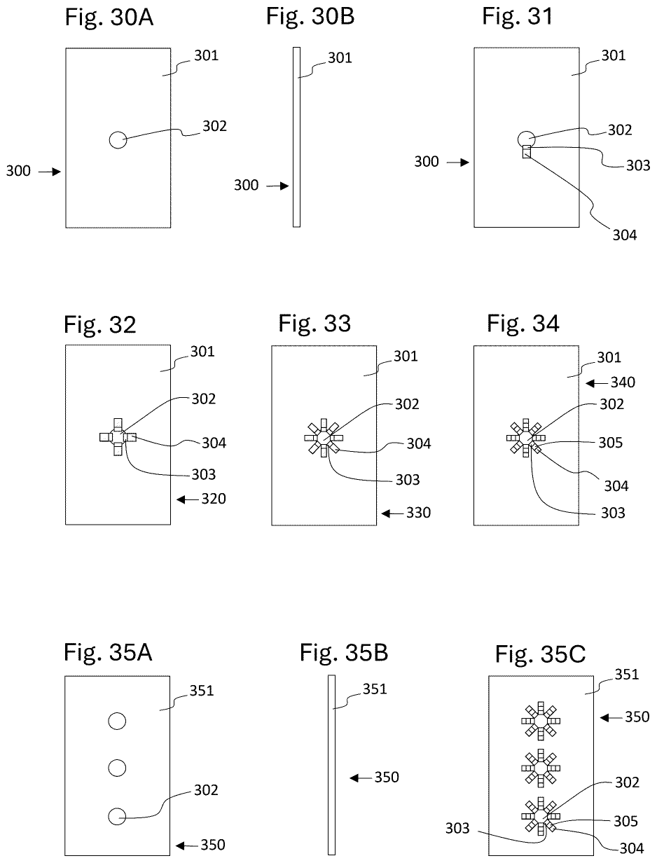

The device 197 introduced in FIG. 21B is designed to incorporate any capillary-driven assay capable of running in a horizontal orientation. FIGS. 30A through 35C show various diagrammatic drawings describing the incorporation of one or more dry-reagent chemistry assays onto the device. FIG. 30A shows a first embodiment of a diagrammatic top view of an assay device 300 comprising a backing 301 and a filter-containing sample application port 302. FIG. 30B shows the diagrammatic side view of the FIG. 30A device. FIG. 30C is a diagrammatic bottom view of the FIG. 30A device showing a dry-reagent chemistry assay pad 304 connected to the bottom side of the filter application port through a transfer membrane 303. The circular design of the application port allows for multiple assay reaction pads, comprising different assays or controls, to be radially placed around it. FIGS. 32 through 34 are diagrammatic bottom views of assay devices incorporating the same backing described in FIG. 30A with additional assay reaction pads radially incorporated around the sample addition port. FIG. 32 shows a device 320 comprising four assays and FIG. 33 shows a device 330 comprising 8 assays. FIG. 34 shows a device 340 incorporating 16 assay reaction pads in which two separate pads, 304 and 305, are placed in tandem along the same transfer membrane 303 with eight of these two-pad structures radially surrounding the application port. FIG. 35A shows the diagrammatic top view of a device 350 comprising three sample ports on a single device, each port capable of being constructed with any of the configurations (1-16 reaction pads) described in FIGS. 30A through 34. FIG. 35B shows a diagrammatic side view of the FIG. 35A device. FIG. 35C shows a diagrammatic bottom view of the FIG. 35A device in which each of the sample ports incorporates 16 reaction pads similar to FIG. 34. For examples 32 through 35C, the multiple reaction pads may be comprised of any combination of assays and controls. For example, in FIG. 35C, for each set of two-pad reaction structures aligned in tandem on a common transfer membrane one of the pads may be an assay targeting a specific analyte while the other pad may be a control pad performing a control reaction. This would result in an assay device having 24 unique assays and 24 control reactions for reach of the assays.

### Example 23: Loading a Horizontal Assay Device onto an Assay Reader

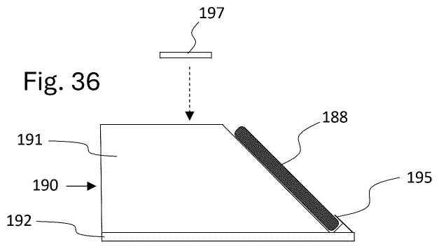

FIG. 36 is a diagrammatic side view demonstrating the basic step loading a horizontal assay device, such as the devices described in FIGS. 29 through 35, onto an assay reader, such as the reader described in FIG. 21. The device 197 is loaded into the accommodating holder of the assay device with the bottom side facing into the reader in the direction shown by the arrow FIG. 23B shows the device after being loaded onto the reader. In a preferred embodiment, the user engages the smartphone screen to indicate that the device has been inserted. In other embodiments, the reader incorporates a sensor which automatically informs the smartphone a device has been inserted. Once the smartphone is engaged, it will prompt the user as to the appropriate time to add sample. In a preferred embodiment, the smartphone will begin collecting images of the device before the user is prompted to add sample.

### Example 24: Adding Sample to a Horizontal Assay Device

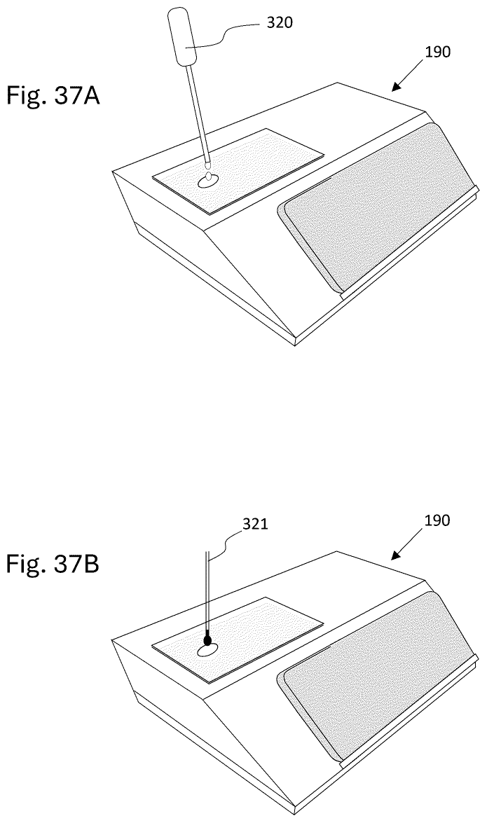

FIG. 37A shows a perspective view of the FIG. 36 horizontal assay device loaded onto an assay reader as a sample is applied to the device sample port using a sample pipet.

FIG. 37B shows a perspective view of the FIG. 36 horizontal assay device loaded onto an assay reader as a blood sample is applied to the device sample port using a blood capillary pipet 321. In a preferred embodiment, the port of the device would contain a matrix designed to separate out red blood cells from plasma, allowing the plasma to flow into the underlying assay component of the device.

### Example 25: Recording an Assay Reaction while Sample is Applied to a Horizontally Oriented Assay Device on an Assay Reader

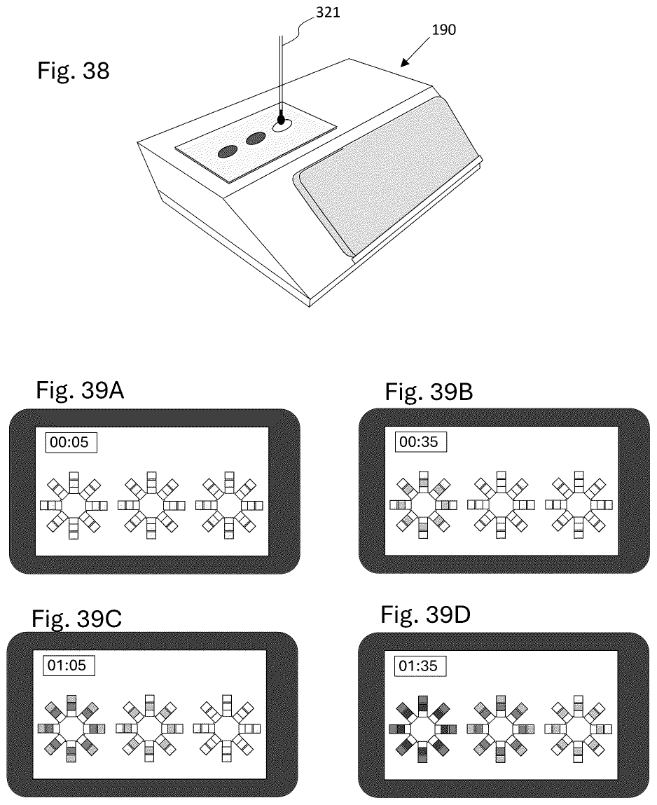

FIG. 38 shows the perspective view of the FIG. 37B assay reader with the FIG. 35 horizontal multi-analyte dry-chemistry assay device loaded onto the reader and in the process of adding a whole blood sample to the three ports of the device. As noted, the smartphone begins recording images of the device prior to addition of the sample, ensuring the capture of the entire assay reaction from initiation. FIGS. 39A through 39D depict the capture of assay reaction images for the dry-chemistry based device. The images depict the smartphone screen of the assay reader at four different time points in a video image recording. FIG. 39A shows the 48 reaction pads, grouped into sets of 18 around the first, second and third sample ports, on the assay device being recorded 5 seconds after prompting the user to add sample. At this early time point, the fluid plasma sample has not yet reached and saturated any of the pads. FIG. 39B shows the pads 35 seconds after prompting, at which point the pads surrounding the first sample port, which was the first to receive sample, begin reacting. FIG. 39C shows the pads 65 seconds after prompting, at which point the pads surrounding the second sample port, which was the second to receive sample, begin reacting. The pads surrounding the first port continue reacting while the pads surrounding the third sample port remain unreacted. FIG. 39D shows the pads 95 seconds after prompting, at which point the pads surrounding the third sample port begin reacting while the pads surrounding the first and second sample ports continue to react. The system will continue recording until the pad that is the last to reach its completion time has completed. Thus, the reader will have recorded for a length of time that captures the complete reaction of all 48 pads irrespective of the time point in which each pad reaction began and ended, and the system will analyze each pad independently. Note that the visualization of the reaction on the smartphone screen is shown to describe the recording process in this example and is not a necessary requirement for the invention. The easy access to the device for sample addition, while simultaneously viewing the prompting on the smartphone screen, underscores the benefits of the assay reader design with the incorporated mirror, enabling both the screen and device to be placed on the same user-facing side of the instrument.

### Example 26: Pseudo-Dry-Reagent Chemistry Assay for Hemoglobin

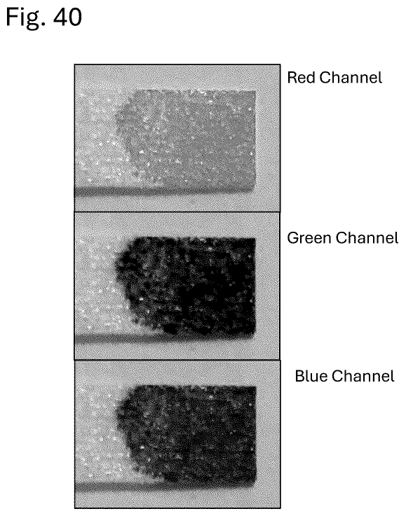

The invention allows for the analysis of capillary-driven assays that incorporate dry-reagent chemistry reactions, wherein colorimetric reactions form on a capillary matrix. In an unexpected discovery, it was found that the method may be applied to the determination of hemoglobin concentration in a small volume of whole blood, such as that obtained from a finger prick, when applied to certain capillary matrices that allow for rapid spreading of the sample with a sufficiently large surface to volume ratio. When collected on these matrices the hemoglobin itself functions as a “pseudo-reagent” providing a measurable color on the matrix which transitions, as the sample dries, in a manner correlating with the hemoglobin concentration of the blood sample. This collected blood sample may then be recorded by a digital camera and analyzed with a machine learning model trained on such samples. In a preferred embodiment, the capillary matrix is a hydrophilic synthetic polymer able to spread the blood sample over a relatively large area (relative to sample volume) such as Ahlstrom® Munksjo 6614 synthetic conjugate pad material. FIG. 40 shows the digital image (three color channels) of a blood sample collected on a 5 mm wide strip of 6614 material. The image was collected approximately one minute after collection from a finger prick. The large surface-to-volume ratio allows for sufficient data with very small samples, such as <5 μl of whole blood. As hemoglobin is, itself, a color pigment, the grayscale value will be proportional to hemoglobin concentration but will also be affected by drying time and spreading, and these variables are addressable with the high dimensional contextualization method described in the invention.

### Example 27: Fourth Processed Input for Lateral Flow Assay Dataset Using Temporal Curves

Examples 3-5 demonstrated progressive improvements in data analysis achieved by incrementally increasing the number of spatial units. Although increasing the number of spatial units tends to improve signal uniformity within each unit, the resulting reduction in unit size may unintentionally reduce the signal-to-noise ratio. A further refinement in the preprocessing strategy is therefore to identify an optimal unit size that balances the total number of spatial units with the signal/noise characteristics of each unit.

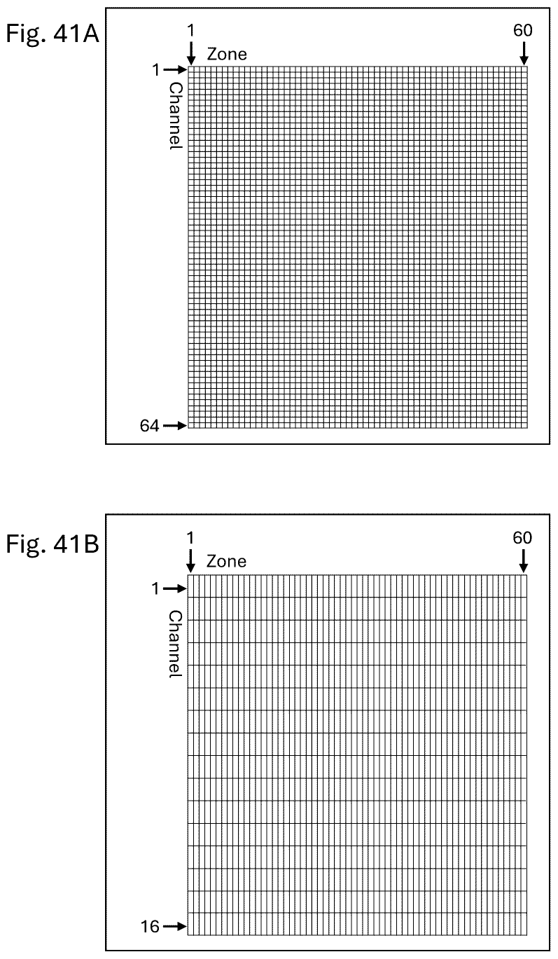

FIGS. 41A and 41B illustrate one such approach. In this example, a digital image ROI from an LFA test 410, consists initially of 3,840 pixels as shown in FIG. 41A. This ROI is segmented into a grid of 960 spatial units 411 as shown in FIG. 41B. Each spatial unit consists of a 1×4-pixel block, arranged to create 16 channels (aligned with the direction of particle flow) and 60 zones. Because each zone is maintained at a width of 1 pixel, the resulting grid preserves very high-resolution gradient features across the binding region of the LFA strip, such as the features described in Example 6.

### Example 28: Fifth Processed Input for a Lateral Flow Assay Dataset Using Temporal Curves

The processing strategy described in Example 27 converted the 3,840-pixel ROI into a fixed set of 16 channels, each channel comprising 60 spatial units of size 1×4 pixels. To further increase the likelihood of generating channels in which the spatial units exhibit optimal signal uniformity, a sliding-window pixel-consolidation approach may be applied.

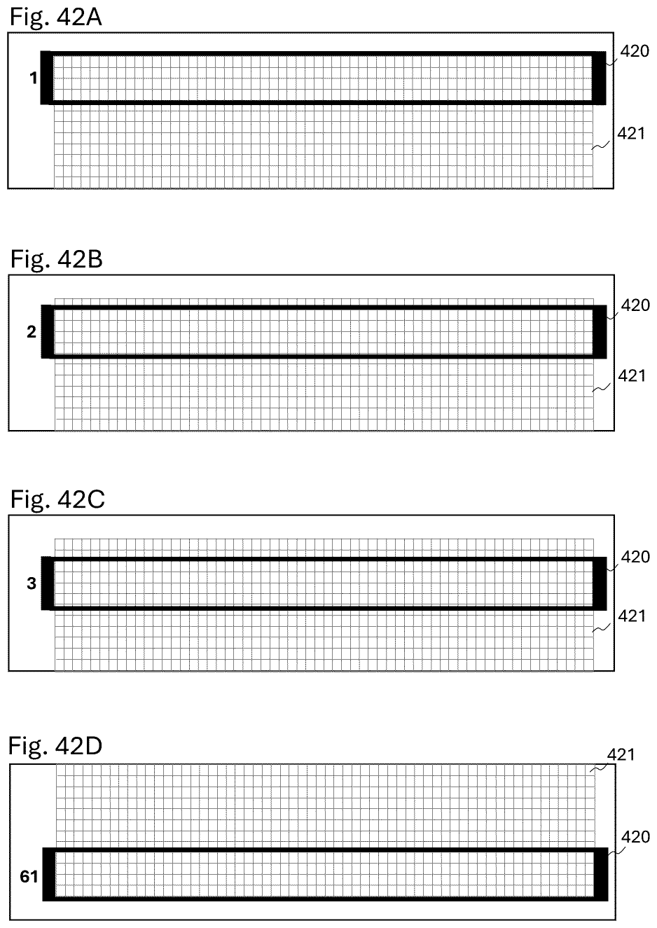

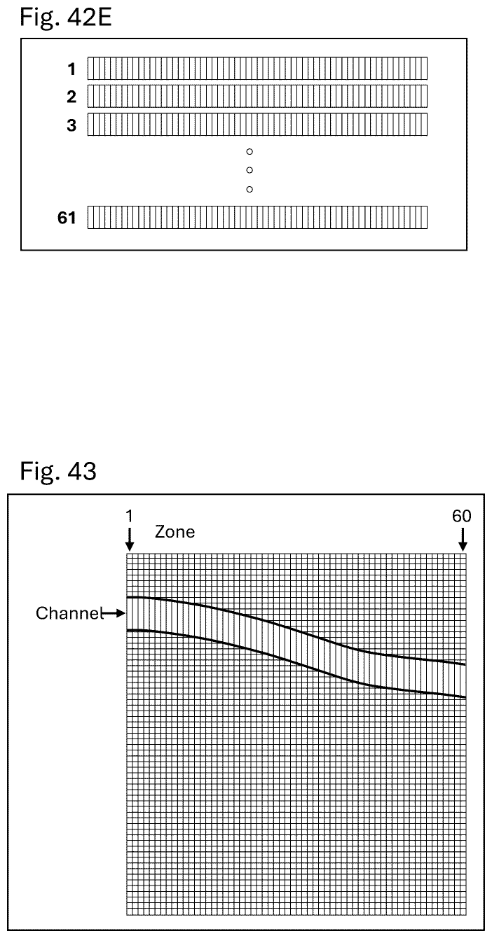

FIG. 42A illustrates a subsection of the FIG. 41A ROI 410 with a 4×60 frame 420 superimposed over the top four 1×60 channels (each originally composed of single-pixel spatial units). This frame defines a processed channel (Channel 1) consisting of sixty 1×4 spatial units formed from those original four channels. FIG. 42B shows the same ROI subsection with the frame shifted downward by one pixel to generate a second processed channel (Channel 2), which overlaps Channel 1 by 75%. FIG. 42C shows the next downward shift, producing Channel 3 with 75% overlap relative to Channel 2. This one-pixel downward sliding continues until the frame reaches the bottom boundary of the ROI, as depicted in FIG. 42D, thereby generating the final processed channel (Channel 61).

FIG. 42E shows these four example channels in their processed form, each containing a sequence of 1×4 spatial units. Channels 4 through 60 are indicated collectively by a vertical sequence of circles denoting these intervening channels. By creating 61 overlapping channels, the preprocessing phase may optionally include a selection step in which a subset of channels is chosen for downstream analysis, retaining only those channels exhibiting strong, uniform signal characteristics.

### Example 29: Sixth Processed Input for a Lateral Flow Assay Dataset Using Temporal Curves

The preprocessing strategies described in Examples 27 and 28 generate linear channels. To further increase the likelihood of producing channels in which the spatial units exhibit optimal signal uniformity, non-linear channel generation may also be employed. This approach accommodates test runs in which a substantial portion of particles travels along a non-linear pathway.

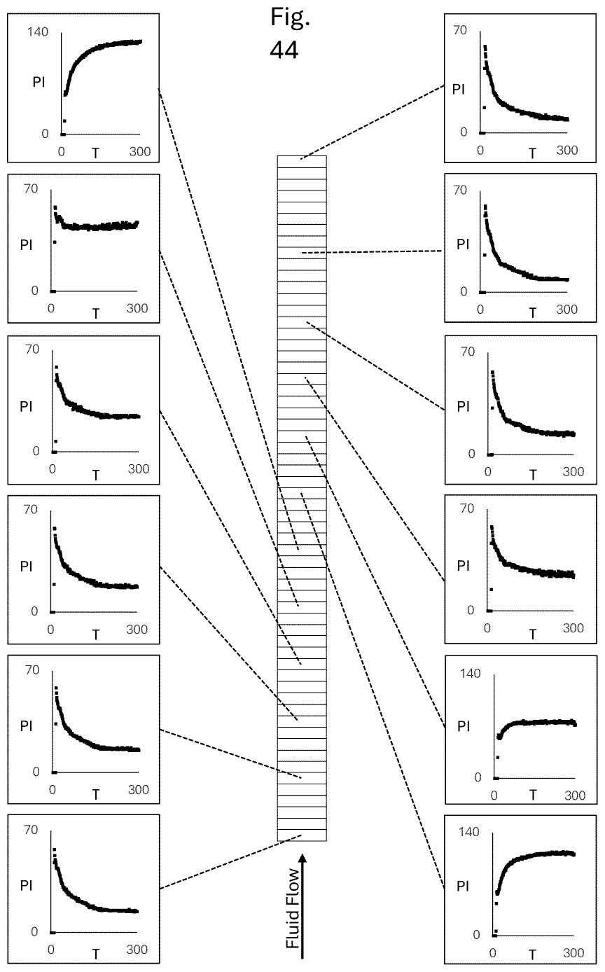

FIG. 43 illustrates the ROI defined in Example 27 410, in which a non-linear channel 430 is constructed to follow a particle population moving through the ROI in such a non-linear fashion. The channel is composed of spatial units consisting of 6 pixels arranged in a 1×6 orientation, and its path progresses across the zones of the ROI with a non-linear trajectory that terminates at a position shifted six pixels downward relative to its starting point.

### Example 30: Lateral Flow Assay Temporal Curve Analysis in a High Resolution ROI Channel

An experiment was conducted to evaluate temporal curve quality within a channel of an ROI when the ROI is processed into a high-resolution collection of spatial units as described in Example 27. A reader was constructed using an Apple® iPhone® 17 smartphone equipped with software to capture digital images of an LFA reaction at 5 frames per second. The smartphone was mounted in a housing that positioned the camera lens approximately 3 cm above a 3-mm-wide methamphetamine competitive immunoassay strip affixed to a plastic card. A negative synthetic urine sample was applied, and the reaction was recorded for 300 seconds.

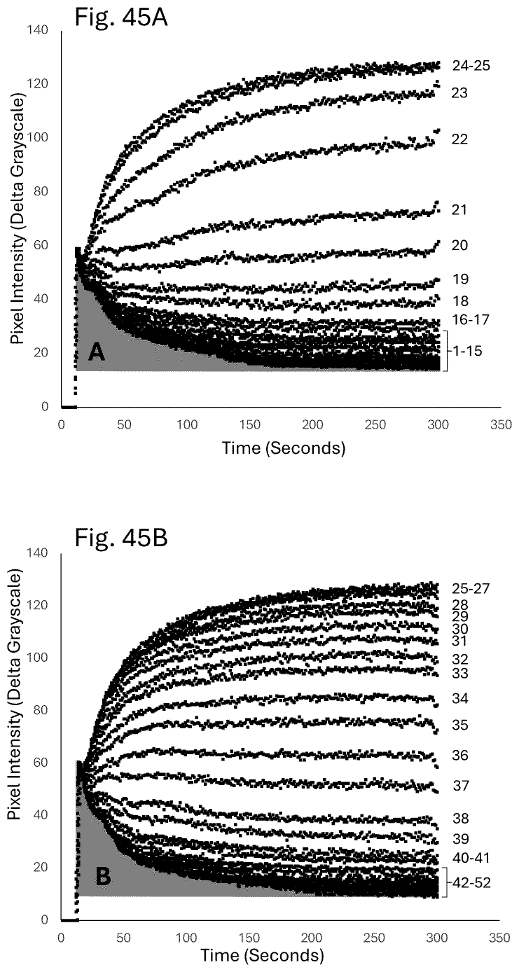

The resulting image stack was processed using an ROI of approximately 3.2×3.0 mm. This ROI was partitioned into a grid of 16 channels and 60 zones, producing 960 spatial units, with each spatial unit corresponding to an area of roughly 50×200 microns on the surface of the LFA strip. Temporal curves were calculated for each spatial unit by determining the delta grayscale or pixel intensity (PI), defined as the grayscale value at each time point minus the grayscale value at time zero (the white, unreacted nitrocellulose). All grayscale values were extracted from the green channel of the smartphone camera, and datapoints were sampled at 1-second intervals. FIG. 44 shows a selected set of 12 scatter plots spanning the length of the channel from zone 1 through zone 60. Each spatial unit displays a temporal curve with the appearance of a smooth quasi-line plot, demonstrating both high resolution and strong signal-to-noise performance despite the extremely small size of the spatial units. FIGS. 45A and 45B plot 52 temporal curves for this channel, with zones 1-25 shown in FIG. 45A and zones 25-52 shown in FIG. 45B (53-60 were omitted as unobservable, fully overlapping 50-52). Because zone 25 corresponds to the peak binding signal, separating the plots into two graphs clearly illustrates the ascending spatial gradient transitioning from the inflow area into the binding area, as well as the descending gradient extending across the test-line zone and continuing into the outflow region.

From a machine-learning standpoint, FIGS. 45A and 45B highlight several important and trainable features. Although the test line appears macroscopically as a single ~1-mm-wide binding region, the current method resolves this region into at least 20-25 discrete binding signals (zones 16-40). The spatial intensity gradient across the test line does not arise from random particle deposition. Rather, it reflects a deterministic kinetic-capture mechanism governed by interactions between flowing labeled particles and immobilized antibodies within the test-line zone. Each resolved signal is therefore produced under a distinct set of micro-conditions, and spatial resolution enables these signals to be contextualized to their specific capture environments. Signals that produce temporal curves outside the visual boundaries of the test line, such as zones 16-22 on the inflow side and zones 34-40 on the outflow side, may also be incorporated into training rather than ignored. As described in Examples 6 and 14, the collective set of gradients may define a secondary signal at low analyte concentrations in competitive assays (and potentially at high analyte concentrations in sandwich assays). Temporal curves additionally enable models to learn from patterns that arise at pre-steady-state time points, including the early 0-100-second segments of the curves. Finally, when the binding-signal temporal curves are plotted together, they form an overlay integrating with the inflow and outflow temporal curves (shaded regions labeled A in FIGS. 45A and B in FIG. 45B), providing supplementary information associated with particle flow through the channel.

### Example 31: Signal/Context Analysis of a Lateral Flow Assay Processed with a High Resolution ROI

Example 29 demonstrated that high-quality temporal curve data can be obtained when an ROI measuring 3.2×3.0 mm on a lateral-flow assay strip is granularized into 960 spatial units. A second experiment was conducted to evaluate the contextual quality of temporal curves at this same level of spatial resolution. For this study, an LFA reaction was selected in which the test line exhibited pronounced color variation across the binding area. Two channels were then chosen based on their peak binding zones exhibiting the darkest and lightest color values, respectively. Within each channel, these binding-zone signals were contextualized using a single inflow temporal curve. The contextualizing temporal curve was taken from a zone positioned in the inflow region directly upstream of the binding area but exhibiting no binding signal (thereby representing particle-flow behavior alone for each respective channel).

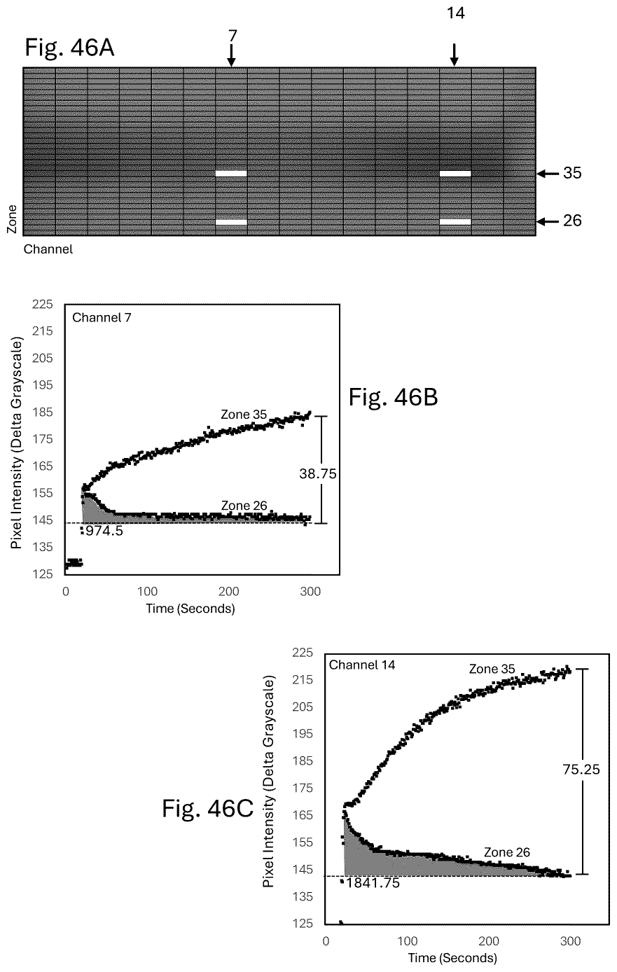

The experimental setup was identical to that described in Example 29, except that the methamphetamine competitive immunoassay strip was replaced with a fentanyl competitive immunoassay strip. FIG. 46A shows a portion of the ROI (zones 24 through 54), encompassing the entire binding area (test line) as well as adjacent inflow and outflow regions. The image corresponds to the final frame at 300 seconds and includes the ROI grid defining all channels and zones. Channels 7 and 14 were identified as having the lightest and darkest peak color values, respectively, with both peaks located at zone 35. Zone 26 was selected in each channel as the contextualizing spatial unit.

FIG. 46B plots the temporal curves for zones 26 and 35 within Channel 7, and FIG. 46C plots the corresponding curves for Channel 14. From these plots, at least two values can be derived, one representing the assay signal and the other representing the assay context. In this experiment, the assay signal was calculated as the difference in pixel intensities between zone 35 and zone 26 at the final time point (300 seconds). The contextualizing value was computed as a quasi-area-under-the-curve for the zone-26 temporal curve by subtracting the pixel intensity at 300 seconds from each earlier datapoint and summing the resulting differences (shaded region).

Channel 14 produced a signal value of 75.25, whereas Channel 7 produced a signal value of 38.75. If considered without contextual information, this level of variation would be unacceptably large for an assay attempting accurate quantitative interpretation, whether selecting channels individually or averaging them. However, the contextual values for the same reaction were 1,841.75 for Channel 14 and 974.5 for Channel 7. Thus, the difference between the two signal values can be understood as arising from channel-specific assay conditions—primarily differences in particle flow—rather than from differences in analyte (fentanyl) concentration.

Although a machine-learning model would apply more sophisticated mathematical operations to achieve such contextualization, the core principle can be seen by comparing the ratios of signal to contextual value:

$$75 \text{.25} / 184 \; 1.75 = 0 \text{.04}$$

$$38.75 / 97 \; 4.5 = 0.04$$

The equivalence of these ratios illustrates that contextualization normalizes channel-specific differences, yielding a consistent underlying assay signal.

### Example 32: Temporal Curve Analysis of Dry-Reagent Chemistry Assay Processed with High-Resolution Spatial Units

An experiment was conducted to evaluate temporal curve quality within the spatial units of an ROI when the ROI is processed into a high-resolution grid for a dry-reagent chemistry assay. A reader was constructed using an Apple® iPhone® 17 smartphone equipped with software to capture digital images of the reaction at 5 frames per second. The smartphone was mounted in a housing that positioned the camera lens approximately 3 cm above a urine creatinine assay device composed of a creatinine reagent strip (sourced from a Wondfo® T-Cup® Compact Multi-Drug Urine Test Cup) affixed to a plastic card containing a trough for sample addition. A positive synthetic urine specimen (100 mg/dL creatinine) was applied, and the reaction was recorded for 200 seconds.

The resulting image stack was processed using an ROI measuring approximately 3.0×2.8 mm centered over the reagent pad. This ROI was divided into a grid of 15 channels and 14 zones, generating 210 spatial units. Each spatial unit corresponded to an area of approximately 200×200 microns on the surface of the pad. Temporal curves were calculated for each spatial unit by determining the delta grayscale or pixel intensity (PI), defined as the grayscale value at each time point minus the grayscale value at time zero (the dry, unreacted pad). All grayscale values were extracted from the green channel of the smartphone camera, and datapoints were sampled at 1-second intervals.

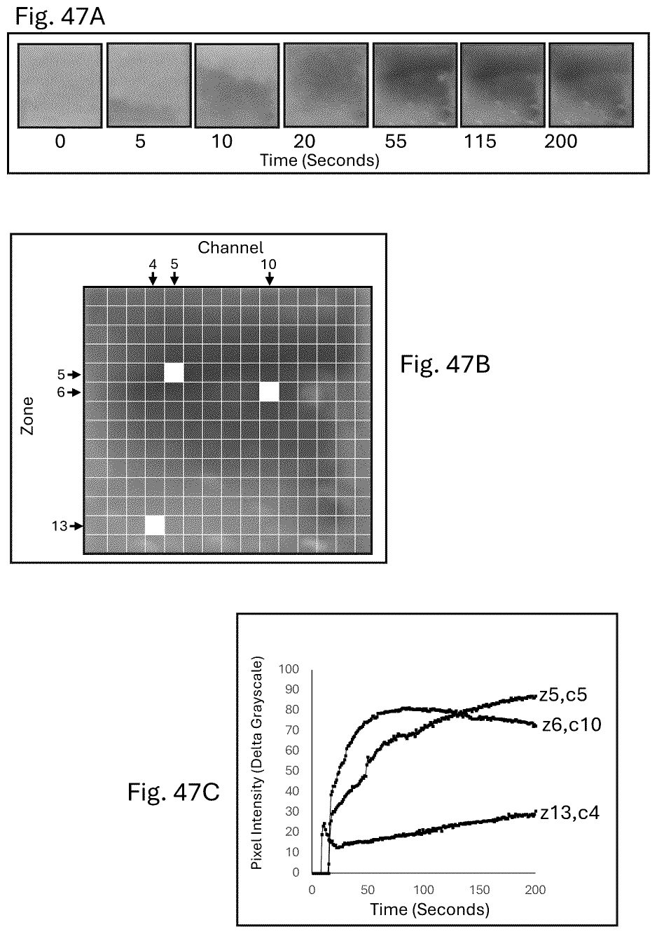

FIG. 47A shows seven ROI images captured at various time points between 0 and 200 seconds. By 5 seconds, initial color development is visible in the lower-right region of the ROI, indicating the first region of the pad to become saturated with sample. As the reaction progresses, color development propagates upward and to the right. By 20 seconds, most of the pad has become saturated. At this stage, contextual assay behavior becomes observable: the lower-right region begins to exhibit a “bleached-out” appearance due to hydrated reagent molecules being mobilized away from this region. By 55 seconds, a darkened streak appears in the upper half of the pad, likely due to a back-flush of particles. Spatial units that appear uniform during the first ~100 seconds subsequently display a secondary color shift associated with the later-stage chemical reaction.

FIG. 47B shows an enlarged image of the ROI at 200 seconds overlaid with the 15×14 spatial-unit grid. Three spatial units exhibiting distinctly different color-development profiles were selected for detailed analysis; these units are shown blanked out in the image. FIG. 47C plots the temporal curves of these selected units. Each spatial unit displays a smooth quasi-line temporal curve, demonstrating high resolution and strong signal-to-noise performance despite the small physical size of each unit. The three curves show that each spatial unit underwent a different set of assay conditions, confirming the need for spatial contextualization rather than signal averaging.

With a sufficiently large set of labeled examples (e.g., all labeled with the known 100 mg/dL creatinine concentration), a machine-learning model may individually contextualize and weight each spatial unit, effectively treating each unit as a distinct micro-reaction. This prevents the excessive variance that would occur if all spatial units were simply averaged together. One example of contextualization is to down-weight or eliminate bleached-out spatial units and compensate for the migration of reagent molecules by appropriately weighting spatial units that received mobilized reagent.

In addition to such weighting strategies, the machine-learning model may exploit the temporal-curve data in several other ways, including but not limited to:
Learning reaction-phase signatures: Identifying characteristic temporal patterns associated with sample entry, saturation, reagent mobilization, back-flush events, and secondary chemical development, each of which provides contextual information useful for concentration prediction.
Modeling spatial-temporal propagation dynamics: Analyzing the relative onset times of temporal curves across neighboring spatial units to infer flow rate, wetting speed, reagent redistribution, and other dynamic behaviors of the assay.
Detecting anomalous or perturbed spatial units: Classifying spatial units into categories (e.g., normal, bleached, back-flushed, chemically shifted) and using these classifications to generate structured weighting maps that guide downstream inference.
Extracting curve-derivative and higher-order kinetic features: Computing features such as initial slope, rate of change, curve curvature, inflection times, and peak velocity, each of which may correlate with assay kinetics or chemical reaction rates.
Learning cross-unit spatial correlations: Modeling how changes in one spatial unit—such as bleaching or reagent depletion—affect responses in downstream units, capturing relationships that arise from reagent transport or diffusion phenomena.
Using early-time kinetic data for rapid inference: Training on the temporal curves' early segments (e.g., the first 10-20 seconds) to enable rapid preliminary analyte estimation before full color development.
Aggregating temporal data at channel or zone levels: Learning higher-order context features by combining temporal information across predefined channels or zones to capture region-level patterns and emergent spatial-temporal behaviors.
Incorporating uncertainty estimation: Quantifying variance across spatial units or within learned latent features to produce an uncertainty score accompanying each predicted analyte concentration.

Together, these approaches allow the machine-learning model to treat the ROI not as a single averaged signal but as a high-dimensional spatial-temporal system, thereby improving precision, robustness, and interpretability of the resulting concentration estimates.

### Example 33: Lateral Flow Assay for Whole Blood Sample Incorporating a Running Buffer

Whole blood samples may be tested on a lateral flow assay device designed to use a running buffer to propagate the assay reaction. Such devices typically include a red blood cell separator that separates plasma from red blood cells, allowing plasma to wick by capillary action toward the conjugate pad and onto the nitrocellulose membrane. Because the volume of plasma obtained from the whole-blood sample is insufficient to fully propagate the reaction, a running buffer is applied after the blood sample is added. The running buffer mixes with the plasma and completes the assay flow.

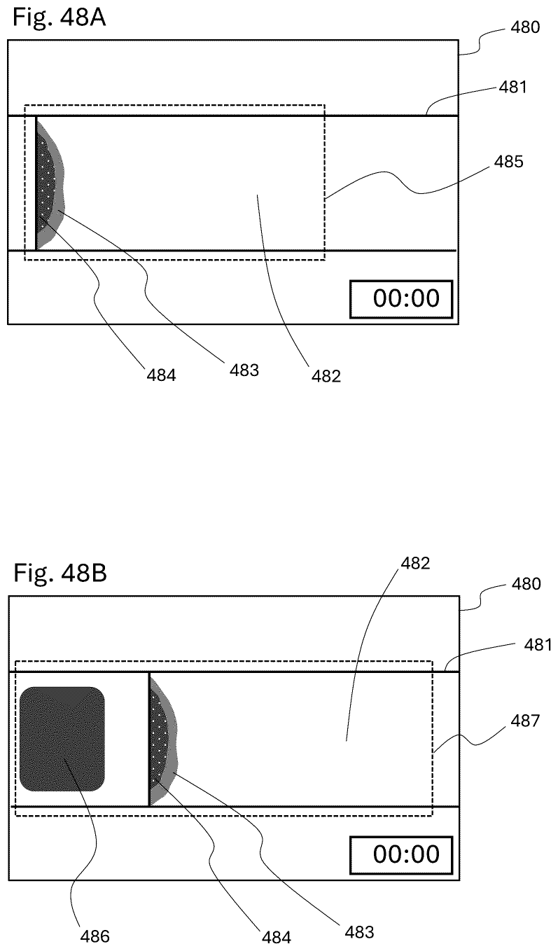

In some embodiments, the whole-blood sample may be applied to the LFA device before the device is placed onto the reader. In additional embodiments, the effective plasma volume may be estimated by expanding the ROI to include a region of the LFA strip in which a portion of the separated plasma is visible. FIG. 48A illustrates this concept, showing the time-0 image of the lateral flow assay strip on the reader 480. The captured image includes the nitrocellulose strip 481 where a section of separated plasma 483 becomes visible along with hydrated nanoparticles 484. In some cases, the nanoparticles may remain outside the field of view at time zero. The ROI is depicted by a dotted outline 485 encompassing both the visible plasma region and the binding area of the strip 482.

For these applications, the machine-learning model may be trained using examples labeled with both the known whole-blood volume and the known hematocrit. In other embodiments, the red blood cell separator may also be included within the captured image to provide additional features relevant to hematocrit estimation. FIG. 48B illustrates such an embodiment, in which the red blood cell separator 486 is incorporated into an expanded ROI 487, supplying additional contextual information for model training.

While several variations of the present invention have been illustrated by way of example in particular embodiments, it is apparent that further embodiments could be developed within the spirit and scope of the present invention. However, it is to be expressly understood that such modifications and adaptations are within the spirit and scope of the present invention, and are inclusive, but not limited to the following appended claims as set forth.

1. A method for determining a quantity of a target substance in a test medium using an assay device having capillary-driven flow, the method comprising:
(a) defining, within the assay device, a region of interest (ROI) associated with development of one or more measurable signals during operation of the assay device;
(b) partitioning the ROI into a plurality of measurement spatial units, each unit representing a spatial subdivision of the ROI;
(c) obtaining, from the plurality of measurement spatial units, a plurality of contextual measurements {Yᵢ}, each contextual measurement Yᵢ representing a measurable property within a corresponding measurement spatial unit and characterizing physical or chemical conditions occurring within the ROI;
(d) identifying, from among the contextual measurements {Yᵢ}, one or more signal measurements comprising contextual measurements that include analyte-dependent information arising during operation of the assay device;
(e) processing the contextual measurements {Yᵢ} and the signal measurements using a machine-learning model configured to identify patterns or relationships within the measurements and to generate one or more intermediate representations;
(f) computing a signal value X for the ROI as a function of the contextual measurements {Yᵢ}, the intermediate representations, and a set of assay-specific parameters C, such that X=f({Yᵢ}, C); and
(g) determining a quantity of the target substance in the test medium based at least in part on the computed signal value X.

2. The method of claim 1, wherein the assay device comprises a porous or semi-porous material configured to transport the test medium at least in part by capillary action, the porous or semi-porous material including one or more of: a lateral-flow membrane, a dry-reagent chemistry pad, a paper-based microfluidic structure, or a chromatographic medium.

3. The method of claim 1, wherein the contextual measurements {Yᵢ} comprise measurements indicative of one or more assay conditions, including at least one of: local fluid transport behavior, reaction kinetics, background or baseline characteristics, membrane wetting dynamics, or variations in physical or chemical properties across the ROI.

4. The method of claim 1, wherein each signal measurement comprises a measurement obtained from a measurement spatial unit in which a detectable change occurs in response to the presence or quantity of the target substance in the test medium.

5. The method of claim 1, further comprising capturing one or more digital images of the assay device during operation of the assay device using a digital camera, the digital images containing data from which the contextual measurements {Yᵢ} and the signal measurements are obtained.

6. The method of claim 5, wherein the digital camera captures a single image or a sequence of images recorded at predetermined time intervals during the assay reaction.

7. The method of claim 5, wherein the digital camera comprises a complementary metal-oxide-semiconductor (CMOS) image sensor or a charge-coupled device (CCD) image sensor configured to convert incident light from the assay device into digital image data.

8. The method of claim 5, wherein the digital camera is integrated into a smartphone configured to record images of the assay device before, during, and after development of the one or more measurable signals within the ROI.

9. The method of claim 8, wherein the smartphone is positioned within an assay reader comprising a housing configured to hold both the smartphone and the assay device in fixed relative positions suitable for image capture, the housing including an interior cavity configured to provide a controlled illumination environment.

10. The method of claim 9, wherein the housing includes a mirror positioned within the interior cavity and arranged such that the smartphone camera records the assay device through a reflection of the mirror, the mirror facilitating a user-accessible orientation of both the smartphone and the assay device.

11. The method of claim 1, wherein the machine learning model comprises one or more of: a convolutional neural network (CNN), a recurrent neural network (RNN), a long short-term memory (LSTM) network, a transformer-based model, a support vector machine (SVM), a random forest, a gradient boosting model, or a hybrid model combining deep learning and classical machine learning components.

12. The method of claim 1, wherein the machine learning model is configured to process spatial features, temporal features, or spatiotemporal features extracted from a sequence of digital images of the assay device.

13. The method of claim 1, wherein the machine learning model comprises a transformer-based architecture configured to apply attention across spatial or temporal dimensions of the digital images to enhance contextual interpretation of the contextual measurements {Yᵢ} and the signal measurements.

14. The method of claim 1, wherein the machine learning model is trained at least in part using a self-supervised learning method that includes selectively masking or occluding portions of one or more regions of the assay device within the training images, thereby enabling the model to infer expected measurements based on contextual relationships within the ROI.

15. The method of claim 1, wherein the machine learning model is trained using synthetic training data generated by algorithmically modifying or simulating assay images to introduce variations in signal intensity, flow pattern characteristics, lighting conditions, noise, occlusions, or spatial distortions.

16. The method of claim 1, wherein the assay device comprises a lateral flow assay including a nitrocellulose membrane, a sample pad, a conjugate pad, and one or more test regions containing immobilized reagents configured to generate at least one of the signal measurements during capillary-driven transport of the test medium.

17. The method of claim 1, wherein the assay device comprises a dry-reagent chemistry assay including one or more porous or absorbent reaction pads containing immobilized reagents configured to undergo a chromogenic, fluorogenic, enzymatic, or colorimetric reaction upon contact with the test medium, the reaction producing one or more signal measurements within the ROI.

18. The method of claim 1, further comprising establishing a confidence range associated with the determined quantity of the target substance, wherein the confidence range is derived at least in part from an evaluation of prediction accuracy of a control region of the assay device.

19. The method of claim 18, wherein the control region comprises a control line of a lateral flow assay, and wherein deviations between predicted values and expected values associated with the control line are used to compute a model confidence score.

20. The method of claim 19, wherein the model confidence score is used to dynamically adjust the confidence range associated with one or more signal measurements corresponding to one or more test regions of the assay device.
